SEQUENCE LISTING (54 sequences)

SEQ ID NO: 1 - protein, 466 aa, Homo sapiens
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - DNA, 1401 bp, Homo sapiens
atggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctaa

SEQ ID NO: 3 - DNA, 1401 bp, Artificial Sequence
  Description of Artificial Sequence Synthetic polynucleotide
atggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaa

SEQ ID NO: 4 - DNA, 1401 bp, Artificial Sequence
  Description of Artificial Sequence Synthetic polynucleotide
atggtgagccaggccctgcgcctgctgtgcctgctgctgggcctgcagggctgcctggccgccggcggcgtggccaaggccagcggcggcgagacccgcgacatgccctggaagcccggcccccaccgcgtgttcgtgacccaggaggaggcccacggcgtgctgcaccgccgccgccgcgccaacgccttcctggaggagctgcgccccggcagcctggagcgcgagtgcaaggaggagcagtgcagcttcgaggaggcccgcgagatcttcaaggacgccgagcgcaccaagctgttctggatcagctacagcgacggcgaccagtgcgccagcagcccctgccagaacggcggcagctgcaaggaccagctgcagagctacatctgcttctgcctgcccgccttcgagggccgcaactgcgagacccacaaggacgaccagctgatctgcgtgaacgagaacggcggctgcgagcagtactgcagcgaccacaccggcaccaagcgcagctgccgctgccacgagggctacagcctgctggccgacggcgtgagctgcacccccaccgtggagtacccctgcggcaagatccccatcctggagaagcgcaacgccagcaagccccagggccgcatcgtgggcggcaaggtgtgccccaagggcgagtgcccctggcaggtgctgctgctggtgaacggcgcccagctgtgcggcggcaccctgatcaacaccatctgggtggtgagcgccgcccactgcttcgacaagatcaagaactggcgcaacctgatcgccgtgctgggcgagcacgacctgagcgagcacgacggcgacgagcagagccgccgcgtggcccaggtgatcatccccagcacctacgtgcccggcaccaccaaccacgacatcgccctgctgcgcctgcaccagcccgtggtgctgaccgaccacgtggtgcccctgtgcctgcccgagcgcaccttcagcgagcgcaccctggccttcgtgcgcttcagcctggtgagcggctggggccagctgctggaccgcggcgccaccgccctggagctgatggtgctgaacgtgccccgcctgatgacccaggactgcctgcagcagagccgcaaggtgggcgacagccccaacatcaccgagtacatgttctgcgccggctacagcgacggcagcaaggacagctgcaagggcgacagcggcggcccccacgccacccactaccgcggcacctggtacctgaccggcatcgtgagctggggccagggctgcgccaccgtgggccacttcggcgtgtacacccgcgtgagccagtacatcgagtggctgcagaagctgatgcgcagcgagccccgccccggcgtgctgctgcgcgcccccttcccctaa

SEQ ID NO: 5 - protein, 591 aa, Artificial Sequence
  Description of Artificial Sequence Synthetic polypeptide
GGSSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - DNA, 1776 bp, Artificial Sequence
  Description of Artificial Sequence Synthetic polynucleotide
ggcggcagcagcggcggcgacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 7 - protein, 603 aa, Artificial Sequence
  Description of Artificial Sequence Synthetic polypeptide
SVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 8 - DNA, 1812 bp, Artificial Sequence
  Description of Artificial Sequence Synthetic polynucleotide
agcgtgagccagaccagcaagctgacccgcgccgagaccgtgttccccgacgtggacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 9 - DNA, 6164 bp, Artificial Sequence
  Description of Artificial Sequence Synthetic polynucleotide
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccgaattcgcggccgctaaacccagctttcttgtacaaagtggcaactttattatacatagttgatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccctcgagttaattaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 10 - DNA, 1733 bp, Artificial Sequence
  Description of Artificial Sequence Synthetic polynucleotide
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattg

SEQ ID NO: 11 - protein, 1057 aa, Artificial Sequence
  Description of Artificial Sequence Synthetic polypeptide
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPGGSSGGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 12 - protein, 1069 aa, Artificial Sequence
  Description of Artificial Sequence Synthetic polypeptide
MVSQALRLLCLLLGLQGCLAAGGVAKASGGETRDMPWKPGPHRVFVTQEEAHGVLHRRRRANAFLEELRPGSLERECKEEQCSFEEAREIFKDAERTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASKPQGRIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFPSVSQTSKLTRAETVFPDVDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 13 - DNA, 3177 bp, Artificial Sequence
  Description of Artificial Sequence Synthetic polynucleotide
atggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaaggcggcagcagcggcggcgacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 14 - DNA, 3213 bp, Artificial Sequence
  Description of Artificial Sequence Synthetic polynucleotide
atggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaaagcgtgagccagaccagcaagctgacccgcgccgagaccgtgttccccgacgtggacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 15 - DNA, 3177 bp, Artificial Sequence
  Description of Artificial Sequence Synthetic polynucleotide
atggtgagccaggccctgcgcctgctgtgcctgctgctgggcctgcagggctgcctggccgccggcggcgtggccaaggccagcggcggcgagacccgcgacatgccctggaagcccggcccccaccgcgtgttcgtgacccaggaggaggcccacggcgtgctgcaccgccgccgccgcgccaacgccttcctggaggagctgcgccccggcagcctggagcgcgagtgcaaggaggagcagtgcagcttcgaggaggcccgcgagatcttcaaggacgccgagcgcaccaagctgttctggatcagctacagcgacggcgaccagtgcgccagcagcccctgccagaacggcggcagctgcaaggaccagctgcagagctacatctgcttctgcctgcccgccttcgagggccgcaactgcgagacccacaaggacgaccagctgatctgcgtgaacgagaacggcggctgcgagcagtactgcagcgaccacaccggcaccaagcgcagctgccgctgccacgagggctacagcctgctggccgacggcgtgagctgcacccccaccgtggagtacccctgcggcaagatccccatcctggagaagcgcaacgccagcaagccccagggccgcatcgtgggcggcaaggtgtgccccaagggcgagtgcccctggcaggtgctgctgctggtgaacggcgcccagctgtgcggcggcaccctgatcaacaccatctgggtggtgagcgccgcccactgcttcgacaagatcaagaactggcgcaacctgatcgccgtgctgggcgagcacgacctgagcgagcacgacggcgacgagcagagccgccgcgtggcccaggtgatcatccccagcacctacgtgcccggcaccaccaaccacgacatcgccctgctgcgcctgcaccagcccgtggtgctgaccgaccacgtggtgcccctgtgcctgcccgagcgcaccttcagcgagcgcaccctggccttcgtgcgcttcagcctggtgagcggctggggccagctgctggaccgcggcgccaccgccctggagctgatggtgctgaacgtgccccgcctgatgacccaggactgcctgcagcagagccgcaaggtgggcgacagccccaacatcaccgagtacatgttctgcgccggctacagcgacggcagcaaggacagctgcaagggcgacagcggcggcccccacgccacccactaccgcggcacctggtacctgaccggcatcgtgagctggggccagggctgcgccaccgtgggccacttcggcgtgtacacccgcgtgagccagtacatcgagtggctgcagaagctgatgcgcagcgagccccgccccggcgtgctgctgcgcgcccccttcccctaaggcggcagcagcggcggcgacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 16 - DNA, 3213 bp, Artificial Sequence
  Description of Artificial Sequence Synthetic polynucleotide
atggtgagccaggccctgcgcctgctgtgcctgctgctgggcctgcagggctgcctggccgccggcggcgtggccaaggccagcggcggcgagacccgcgacatgccctggaagcccggcccccaccgcgtgttcgtgacccaggaggaggcccacggcgtgctgcaccgccgccgccgcgccaacgccttcctggaggagctgcgccccggcagcctggagcgcgagtgcaaggaggagcagtgcagcttcgaggaggcccgcgagatcttcaaggacgccgagcgcaccaagctgttctggatcagctacagcgacggcgaccagtgcgccagcagcccctgccagaacggcggcagctgcaaggaccagctgcagagctacatctgcttctgcctgcccgccttcgagggccgcaactgcgagacccacaaggacgaccagctgatctgcgtgaacgagaacggcggctgcgagcagtactgcagcgaccacaccggcaccaagcgcagctgccgctgccacgagggctacagcctgctggccgacggcgtgagctgcacccccaccgtggagtacccctgcggcaagatccccatcctggagaagcgcaacgccagcaagccccagggccgcatcgtgggcggcaaggtgtgccccaagggcgagtgcccctggcaggtgctgctgctggtgaacggcgcccagctgtgcggcggcaccctgatcaacaccatctgggtggtgagcgccgcccactgcttcgacaagatcaagaactggcgcaacctgatcgccgtgctgggcgagcacgacctgagcgagcacgacggcgacgagcagagccgccgcgtggcccaggtgatcatccccagcacctacgtgcccggcaccaccaaccacgacatcgccctgctgcgcctgcaccagcccgtggtgctgaccgaccacgtggtgcccctgtgcctgcccgagcgcaccttcagcgagcgcaccctggccttcgtgcgcttcagcctggtgagcggctggggccagctgctggaccgcggcgccaccgccctggagctgatggtgctgaacgtgccccgcctgatgacccaggactgcctgcagcagagccgcaaggtgggcgacagccccaacatcaccgagtacatgttctgcgccggctacagcgacggcagcaaggacagctgcaagggcgacagcggcggcccccacgccacccactaccgcggcacctggtacctgaccggcatcgtgagctggggccagggctgcgccaccgtgggccacttcggcgtgtacacccgcgtgagccagtacatcgagtggctgcagaagctgatgcgcagcgagccccgccccggcgtgctgctgcgcgcccccttcccctaaagcgtgagccagaccagcaagctgacccgcgccgagaccgtgttccccgacgtggacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 17 - DNA, 3177 bp, Artificial Sequence
  Description of Artificial Sequence Synthetic polynucleotide
atggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctaaggcggcagcagcggcggcgacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 18 - DNA, 3213 bp, Artificial Sequence
  Description of Artificial Sequence Synthetic polynucleotide
atggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctaaagcgtgagccagaccagcaagctgacccgcgccgagaccgtgttccccgacgtggacgcccacaagagcgaggtggcccaccgcttcaaggacctgggcgaggagaacttcaaggccctggtgctgatcgccttcgcccagtacctgcagcagtgccccttcgaggaccacgtgaagctggtgaacgaggtgaccgagttcgccaagacctgcgtggccgacgagagcgccgagaactgcgacaagagcctgcacaccctgttcggcgacaagctgtgcaccgtggccaccctgcgcgagacctacggcgagatggccgactgctgcgccaagcaggagcccgagcgcaacgagtgcttcctgcagcacaaggacgacaaccccaacctgccccgcctggtgcgccccgaggtggacgtgatgtgcaccgccttccacgacaacgaggagaccttcctgaagaagtacctgtacgagatcgcccgccgccacccctacttctacgcccccgagctgctgttcttcgccaagcgctacaaggccgccttcaccgagtgctgccaggccgccgacaaggccgcctgcctgctgcccaagctggacgagctgcgcgacgagggcaaggccagcagcgccaagcagcgcctgaagtgcgccagcctgcagaagttcggcgagcgcgccttcaaggcctgggccgtggcccgcctgagccagcgcttccccaaggccgagttcgccgaggtgagcaagctggtgaccgacctgaccaaggtgcacaccgagtgctgccacggcgacctgctggagtgcgccgacgaccgcgccgacctggccaagtacatctgcgagaaccaggacagcatcagcagcaagctgaaggagtgctgcgagaagcccctgctggagaagagccactgcatcgccgaggtggagaacgacgagatgcccgccgacctgcccagcctggccgccgacttcgtggagagcaaggacgtgtgcaagaactacgccgaggccaaggacgtgttcctgggcatgttcctgtacgagtacgcccgccgccaccccgactacagcgtggtgctgctgctgcgcctggccaagacctacgagaccaccctggagaagtgctgcgccgccgccgacccccacgagtgctacgccaaggtgttcgacgagttcaagcccctggtggaggagccccagaacctgatcaagcagaactgcgagctgttcgagcagctgggcgagtacaagttccagaacgccctgctggtgcgctacaccaagaaggtgccccaggtgagcacccccaccctggtggaggtgagccgcaacctgggcaaggtgggcagcaagtgctgcaagcaccccgaggccaagcgcatgccctgcgccgaggactacctgagcgtggtgctgaaccagctgtgcgtgctgcacgagaagacccccgtgagcgaccgcgtgaccaagtgctgcaccgagagcctggtgaaccgccgcccctgcttcagcgccctggaggtggacgagacctacgtgcccaaggagttcaacgccgagaccttcaccttccacgccgacatctgcaccctgagcgagaaggagcgccagatcaagaagcagaccgccctggtggagctggtgaagcacaagcccaaggccaccaaggagcagctgaaggccgtgatggacgacttcgccgccttcgtggagaagtgctgcaaggccgacgacaaggagacctgcttcgccgaggagggcaagaagctggtggccgccagccaggccgccctgggcctgtaa

SEQ ID NO: 19 - DNA, 6 bp, Artificial Sequence
  Description of Artificial Sequence Synthetic oligonucleotide
gccacc

SEQ ID NO: 20 - DNA, 522 bp, Artificial Sequence
  Description of Artificial Sequence Synthetic polynucleotide
tcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 21 - DNA, 798 bp, Artificial Sequence
  Description of Artificial Sequence Synthetic polynucleotide
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 22 - DNA, 10784 bp, Artificial Sequence
  Description of Artificial Sequence Synthetic polynucleotide
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgccaccatggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgccaccatggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaagaattcgtcgacatcgattcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcctcgaggcggccgctaacgagagcataatattgatatgtgccaaagttgtttctgactgactaataagtataatttgtttctattatgtataggttaagctaattacttattttataatacaacatgactgtttttaaagtacaaaataagtttatttttgtaaaagagagaatgtttaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaataatagtttctaatttttttattattcagcctgctgtcgtgaataccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggtacctcgcgcgacttggtttgccattctttagcgcgcgtcgcgtcacacagcttggccacaatgtggtttttgtcaaacgaagattctatgacgtgtttaaagtttaggtcgagtaaagcgcaaatctttt

SEQ ID NO: 23 - DNA, 3968 bp, Artificial Sequence
  Description of Artificial Sequence Synthetic polynucleotide
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgccaccatggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 24 - DNA, 2996 bp, Artificial Sequence
  Description of Artificial Sequence Synthetic polynucleotide
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgccaccatggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaagaattcgtcgacatcgattcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 25 - DNA, 6993 bp, Artificial Sequence
  Description of Artificial Sequence Synthetic polynucleotide
ctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgccaccatggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggatccccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgccaccatggtgtcacaggccctgagactgctgtgtctgctgctgggactgcagggatgtttggctgctggcggagtggctaaagcctctggcggagagacaagagacatgccctggaagcctggacctcacagagtgttcgtgacccaagaggaagcccacggcgttctgcacagaagaagaagggccaacgccttcctggaagaactgaggcctggctctctggaacgcgagtgcaaagaggaacagtgcagcttcgaggaagccagagagatcttcaaggacgccgagcggaccaagctgttctggatcagctacagcgacggcgaccagtgtgccagctctccttgtcagaatggcggcagctgcaaggaccagctgcagagctacatctgcttttgcctgcctgccttcgagggcagaaactgcgagacacacaaggacgaccagctgatctgcgtgaacgagaacggcggctgcgagcagtactgtagcgatcacacaggcaccaagcggagctgcagatgtcacgagggctattccctgctggccgatggcgttagctgtacccctaccgtggaatacccctgcggcaagatccccatcctggaaaagagaaacgccagcaagccccagggcagaatcgttggcggcaaagtgtgccctaagggcgagtgtccttggcaggttctgctgcttgtgaatggcgctcagctgtgtggcggcaccctgatcaataccatctgggtcgtgtccgccgctcactgcttcgacaagatcaagaactggcggaacctgatcgccgtgctgggagagcacgatctgtctgaacacgatggcgacgagcagtctcggagagtggcccaagtgatcatccccagcacctatgtgcccggcaccaccaatcacgatatcgccctgctcagactgcaccagcctgtggtgctgacagatcacgtggtgcctctgtgcctgccagagaggacctttagcgagagaaccctggccttcgtgcggttctctctggtgtctggatggggccagctgctggatagaggcgctacagctctggaactgatggtgctgaacgtgcccagactgatgacccaggattgcctgcagcagagcagaaaagtgggcgacagccccaacatcaccgagtacatgttctgcgccggctactccgacggcagcaaggatagctgtaaaggcgattctggcggccctcacgccacacactatagaggcacctggtatctgaccggcatcgtgtcttggggacagggctgtgctacagtgggccactttggcgtgtacaccagagtgtcccagtacatcgagtggctgcagaaactcatgcggagcgagcctagacctggcgtgttgctgagagccccttttccttaagaattcgtcgacatcgattcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 26 - protein, 12 aa, Artificial Sequence
  Description of Artificial Sequence Synthetic peptide
SWELYYPLRANL

SEQ ID NO: 27 - protein, 5 aa, Artificial Sequence
  Description of Artificial Sequence Synthetic peptide
HAVDI

SEQ ID NO: 28 - protein, 4 aa, Artificial Sequence
  Description of Artificial Sequence Synthetic peptide
DGEA

SEQ ID NO: 29 - protein, 6 aa, Artificial Sequence
  Description of Artificial Sequence Synthetic peptide
FYFDLR

SEQ ID NO: 30 - protein, 6 aa, Artificial Sequence
  Description of Artificial Sequence Synthetic peptide
  MOD_RES (3)..(3): 4-hydroxyproline
GFPGER

SEQ ID NO: 31 - protein, 37 aa, Artificial Sequence
  Description of Artificial Sequence Synthetic polypeptide
  MOD_RES (19)..(19): 4-hydroxyproline
PGPPGPPGPPGPPGPPGFPGERGPPGPPGPPGPPGPP

SEQ ID NO: 32 - protein, 4 aa, Artificial Sequence
  Description of Artificial Sequence Synthetic peptide
VAPG

SEQ ID NO: 33 - protein, 3 aa, Artificial Sequence
  Description of Artificial Sequence Synthetic peptide
RGD

SEQ ID NO: 34 - protein, 3 aa, Artificial Sequence
  Description of Artificial Sequence Synthetic peptide
GPR

SEQ ID NO: 35 - protein, 6 aa, Artificial Sequence
  Description of Artificial Sequence Synthetic peptide
KQAGDV

SEQ ID NO: 36 - protein, 5 aa, Artificial Sequence
  Description of Artificial Sequence Synthetic peptide
PHSRN

SEQ ID NO: 37 - protein, 15 aa, Artificial Sequence
  Description of Artificial Sequence Synthetic peptide
PHSRNGGGGGGRGDS

SEQ ID NO: 38 - protein, 4 aa, Artificial Sequence
  Description of Artificial Sequence Synthetic peptide
REDV

SEQ ID NO: 39 - protein, 5 aa, Artificial Sequence
  Description of Artificial Sequence Synthetic peptide
IKVAV

SEQ ID NO: 40 - protein, 17 aa, Artificial Sequence
  Description of Artificial Sequence Synthetic peptide
SRARKQAASIKVAVADR

SEQ ID NO: 41 - protein, 3 aa, Artificial Sequence
  Description of Artificial Sequence Synthetic peptide
LRE

SEQ ID NO: 42 - protein, 6 aa, Artificial Sequence
  Description of Artificial Sequence Synthetic peptide
KQLREQ

SEQ ID NO: 43 - protein, 5 aa, Artificial Sequence
  Description of Artificial Sequence Synthetic peptide
YIGSR

SEQ ID NO: 44 - protein, 7 aa, Artificial Sequence
  Description of Artificial Sequence Synthetic peptide
SVVYGLR

SEQ ID NO: 45 - protein, 8 aa, Artificial Sequence
  Description of Artificial Sequence Synthetic peptide
AEIDGIEL

SEQ ID NO: 46 - protein, 4 aa, Artificial Sequence
  Description of Artificial Sequence Synthetic peptide
VTCG

SEQ ID NO: 47 - protein, 5 aa, Artificial Sequence
  Description of Artificial Sequence Synthetic peptide
SVTCG

SEQ ID NO: 48 - protein, 5 aa, Artificial Sequence
  Description of Artificial Sequence Synthetic peptide
RGDSP

SEQ ID NO: 49 - protein, 6 aa, Artificial Sequence
  Description of Artificial Sequence Synthetic peptide
GRGDSP

SEQ ID NO: 50 - protein, 4 aa, Artificial Sequence
  Description of Artificial Sequence Synthetic peptide
GRGD

SEQ ID NO: 51 - protein, 7 aa, Artificial Sequence
  Description of Artificial Sequence Synthetic peptide
GGRGDSP

SEQ ID NO: 52 - protein, 8 aa, Artificial Sequence
  Description of Artificial Sequence Synthetic peptide
GGGRGDSP

SEQ ID NO: 53 - protein, 6 aa, Artificial Sequence
  Description of Artificial Sequence Synthetic peptide
  MISC_FEATURE (1)..(3): This region may encompass 1-3 residues
GGGRGD

SEQ ID NO: 54 - protein, 8 aa, Artificial Sequence
  Description of Artificial Sequence Synthetic peptide
  MISC_FEATURE (1)..(3): This region may encompass 1-3 residues
GGGRGDSP

(19) United States
(12) Patent Application Publication
(10) Pub. No.: US 2023/0123802 A1
(43) Pub. Date: Apr. 20, 2023

(54) COMPOSITIONS, DEVICES, AND METHODS FOR FACTOR VII THERAPY

(71) Applicant: SIGILON THERAPEUTICS, INC., Cambridge, MA (US)

(72) Inventors: Lauren Emily Barney, Cambridge, MA (US); Michael Beauregard, Boston, MA (US); Guillaume Carmona, Cambridge, MA (US); Francisco Caballero Gonzalez, Brookline, MA (US); Richard Heidebrecht, Somerville, MA (US); Erika Ellen Johnston, Cambridge, MA (US); Robert James Miller, East Bridgewater, MA (US); Owen O'Connor, Raynham, MA (US); Matthias Alexander Oberli, Cambridge, MA (US); David Peritt, Skokie, IL (US); Jared A. Sewell, Somerville, MA (US); Devyn McKinley Smith, Barrington, RI (US); Omid Veiseh, Bellaire, TX (US); Paul Kevin Wotton, Boston, MA (US); Zoe Yin, Boston, MA (US)

(21) Appl. No.: 17/598,154

(22) PCT Filed: Mar. 27, 2020
(86) PCT No.: PCT/US20/25511

§ 371 Date: Sep. 24, 2021

Related U.S. Application Data
(60) Provisional application No. 62/824,963, filed on Mar. 27, 2019.
(60) Provisional application No. 62/907,386, filed on Sep. 27, 2019.

(51) Int. Cl. A61K 35/30; A61K 9/48; C12N 9/64; C12N 15/52; C12N 5/079

(52) U.S. Cl. CPC A61K 35/30; A61K 9/4816; A61K 9/4891; C12N 9/6437; C12Y 304/21021; C12N 15/52; C12N 5/0621; C12N 2510/00

(57) ABSTRACT

Described herein are RPE cells engineered to secrete a FVII protein, as well as compositions, pharmaceutical preparations, and implantable devices comprising the engineered RPE cells, and methods of making and using the same for treating a patient with hemophilia or FVII deficiency.

### CLAIM OF PRIORITY

This application is a U.S. National Phase Application under 35 U.S.C. § 371 of International Application No. PCT/US2020/025511, filed Mar. 27, 2020, which claims priority to U.S. Provisional Application No. 62/824,963, filed Mar. 27, 2019, and U.S. Provisional Application No. 62/907,386, filed Sep. 27, 2019. The disclosure of each of the foregoing applications is incorporated herein by reference in its entirety.

### SEQUENCE LISTING

The instant application contains a Sequence Listing which has been submitted electronically in ASCII format and is hereby incorporated by reference in its entirety. Said ASCII copy, created on Mar. 25, 2020, is named S2225-7029WO_SL.txt and is 122,698 bytes in size.

### BACKGROUND

Factor VII (FVII) plays a pivotal role in blood coagulation, a series of reactions in which plasma inactive zymogens are converted into active enzymes and resulting in the formation of an insoluble fibrin clot. The inactive form of FVII is expressed in the liver and secreted into the blood as a single chain zymogen. Upon injury to a blood vessel, tissue factor (TF) is exposed to circulating FVII and when complexed with TF, FVII is activated to FVIIa by several different proteases and the factor FVIIa:TF complex catalyzes the conversion of both factor IX (FIX) and factor X (FX) into their activated forms FIXa and FXa, respectively, which leads to thrombin generation and subsequent formation of fibrin clots.

Factor VII deficiency is a rare bleeding disorder characterized by a deficiency or reduced activity of FVII, with an estimated incidence of 1 in 300,000 to 1 in 500,000 people. Congenital FVII deficiency is caused by mutations in the F7 gene. Acquired FVII deficiency can result from severe liver disease, sepsis or vitamin K deficiency, as well certain drugs such as warfarin.

A recombinant activated FVII protein (rFVIIa) is approved for promoting hemostasis in individuals with hemophilia who have antibody inhibitors to FVIII and FIX, patients with acquired hemophilia and patients with congenital FVII deficiency. However, rFVIIa has a short half-life and thus multiple doses in a short time period are required to manage active bleeding episodes in hemophilia patients. Thus, additional FVII therapies are desirable.

### SUMMARY

Described herein is a retinal pigment epithelial (RPE) cell that is engineered to express and secrete FVII, as well as compositions, pharmaceutical products, medical devices comprising the engineered RPE cell, and methods of making and using the same. In some embodiments, the compositions, products and devices comprising the engineered RPE cell are configured to mitigate the foreign body response when administered to, e.g., placed inside, a mammalian subject.

In one aspect, the present disclosure features an isolated polynucleotide comprising a promoter operably linked to a precursor FVII coding sequence. In an embodiment, the promoter sequence consists essentially of a nucleotide sequence that is identical to, or substantially identical to, SEQ ID NO:10 (nucleotides 337-2069 of the sequence shown in FIG. 6B). In an embodiment, the precursor FVII coding sequence is codon optimized for expression in mammalian cells. In an embodiment, the precursor FVII coding sequence encodes a FVII fusion protein. In an embodiment, the FVII fusion protein comprises an amino acid sequence encoding a non-FVII polypeptide operably linked to the C-terminus of the FVII amino acid sequence. In an embodiment, the non-FVII polypeptide confers a beneficial property to the fusion protein, e.g., increases the amount of protein expressed and/or secreted or extends the half-life in vivo. In an embodiment, the FVII fusion protein comprises an amino acid sequence for a mammalian albumin, e.g., a human albumin, which is connected to the C-terminus of the FVII amino acid sequence via a peptide linker. In an embodiment, the peptide linker is proteolytically cleavable. In an embodiment, the precursor FVII codon-optimized coding sequence is SEQ ID NO:3 or SEQ ID NO:4. In an embodiment, the codon-optimized FVII sequence (e.g., SEQ ID NO:3 or SEQ ID NO:4) is operably linked to SEQ ID NO:6 or SEQ ID NO:8. In an embodiment, the isolated polynucleotide is provided as one of the strands in a double-stranded DNA molecule, e.g., in an expression vector.

In another aspect, the present disclosure provides an engineered RPE cell comprising an exogenous nucleotide sequence, which comprises a promoter sequence operably linked to a precursor FVII coding sequence. In an embodiment, the exogenous nucleotide sequence comprises an extrachromosomal expression vector. In an embodiment, the exogenous nucleotide sequence is integrated into at least one location in the genome of the RPE cell, e.g., ARPE-19 cell.

In yet another aspect, the present disclosure provides a device comprising at least one cell-containing compartment which comprises an engineered RPE cell described herein or a plurality of such cells. In some embodiments, the compositions, products and devices comprise a polymer composition encapsulating the engineered RPE cell(s). In an embodiment, the encapsulating polymer composition at least one cell binding-substance (CBS), e.g., a cell binding peptide, e.g., RGD (SEQ ID NO:33) or RGDSP (SEQ ID NO:48). In an embodiment, the encapsulating polymer composition comprises an alginate covalently modified with GRGDSP (SEQ ID NO:49). In some embodiments, the device further comprises at least one means for mitigating the foreign body response (FBR) when the device is placed inside a subject. In an embodiment, the means for mitigating the FBR comprises an afibrotic compound, as defined herein, disposed on an exterior surface of the device and/or within a barrier compartment surrounding the cell-containing compartment. In an embodiment, the afibrotic compound is a compound of Formula (I):

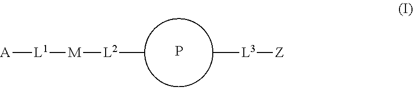

or a pharmaceutically acceptable salt thereof, wherein the variables A, L¹, M, L², P, L³, and Z, as well as related subvariables, are defined herein. In some embodiments, the compound of Formula (I) or a pharmaceutically acceptable salt thereof (e.g., Formulas (I-a), (I-b), (I-c), (I-d), (I-e), (I-f), (II), (II-a), (III), (III-a), (III-b), (III-c), or (III-d)) is a compound described herein, including for example, one of the compounds shown in Table 3 herein. In an embodiment, the afibrotic compound is Compound 100, Compound 101 or Compound 102 shown in Table 3.

In one aspect, a device of the disclosure is a 2-compartment hydrogel capsule (e.g., a microcapsule (less than 1 mm in diameter) or a millicapsule (at least 1 mm in diameter)) in which a cell-containing compartment (e.g., the inner compartment) comprising a plurality of live engineered RPE cells (and optionally one or more cell binding substances) is surrounded by a barrier compartment comprising an afibrotic polymer (e.g., the outer compartment). In an embodiment, the afibrotic compound is a compound of Formula (I). In an embodiment, the hydrogel capsule is a spherical capsule.

In another aspect, the present disclosure features a preparation (e.g., a composition) comprising a plurality (at least any of 3, 6, 12, 25, 50 or more) of an RPE cell-containing device described herein. In some embodiments, the preparation is a pharmaceutically acceptable composition.

In another aspect, the present disclosure features a method of making or manufacturing a device comprising a plurality of RPE cells engineered to express and secrete FVII. In some embodiments, the method comprises providing the plurality of engineered RPE cells and disposing the plurality of RPE cells in an enclosing component, e.g., a cell-containing compartment of the device as described herein. In some embodiments, the enclosing component comprises a flexible polymer (e.g., PLA, PLG, PEG, CMC, or a polysaccharide, e.g., alginate). In some embodiments, the enclosing component comprises an inflexible polymer or metal housing. In some embodiments, the surface of the device is chemically modified, e.g., with a compound of Formula (I) as described herein.

In another aspect, the present disclosure features a method of evaluating an engineered RPE cell or a device described herein. In some embodiments, the method comprises providing the engineered RPE cell or device and evaluating a structural or functional parameter of the RPE cell or device. In some embodiments, the method comprises evaluating the engineered RPE cell or device for one or more of a) cell viability and b) amount of FVII produced. In some embodiments, the evaluation is performed at least 1, 5, 10, 20, 30, 60, 90 or 120 days after (i) formation of the device (or preparation of devices) or (ii) administration of the device (or preparation of devices) to a subject. In an embodiment, the evaluation further comprises assessing the amount of fibrosis and/or structural integrity of the device (or devices within a preparation) at least 30, 60, 90 or 120 days after administration to the subject. In some embodiments, the subject is a mammal (e.g., a mouse, a human).

In another aspect, the present disclosure features a method of treating a subject in need of FVII replacement therapy (e.g., a patient with FVII deficiency, a patient with hemophilia who has inhibitors to FVIII and/or FIX) comprising administering to the subject a device or device preparation comprising an RPE cell engineered to express and secrete FVII, as described herein. In some embodiments, the administering step comprises placing into the subject a pharmaceutically acceptable preparation comprising a plurality of devices, each of which has the ability to produce FVII. In some embodiments, the device or device preparation is administered to, placed in, or provided to a site other than the central nervous system, brain, spinal column, eye, or retina. In some embodiments, the implantable element is administered to, placed in, or injected in the peritoneal cavity (e.g., the lesser sac), the omentum, or the subcutaneous fat of a subject. In an embodiment, the method further comprises measuring the amount or activity of FVII present in a tissue sample removed from the subject, e.g., in plasma separated from a blood sample, a liver biopsy. In an embodiment, the tissue sample is removed at 15, 30, 60 or 120 days. In some embodiments, the subject is a human.

The details of one or more embodiments of the disclosure are set forth herein. Other features, objects, and advantages of the disclosure will be apparent from the Detailed Description, the Figures, the Examples, and the Claims.

### BRIEF DESCRIPTION OF THE DRAWINGS

FIG. 1 shows the amino acid sequence (FIG. 1A, SEQ ID NO:1) and nucleotide coding sequence (FIG. 1B, SEQ ID NO:2) for a wild-type human precursor FVII protein expressed by an exemplary engineered human RPE cell of the disclosure, with underlining indicating the amino acid and coding sequences for the signal peptide.

FIG. 2 shows an exemplary codon-optimized nucleotide sequence (SEQ ID NO:3) encoding the wild-type precursor human FVII protein shown in FIG. 1, with underlining indicating the coding sequence for the signal peptide.

FIG. 3 shows another exemplary codon-optimized nucleotide sequence (SEQ ID NO:4) encoding the wild-type precursor human FVII protein shown in FIG. 1, with underlining indicating the coding sequence for the signal peptide.

FIG. 4 shows the amino acid sequence (FIG. 4A, SEQ ID NO:5) and nucleotide coding sequence (FIG. 4B, SEQ ID NO:6) for the albumin portion of an exemplary FVII fusion protein expressed by an exemplary engineered human RPE cell of the disclosure, with the amino acid and nucleotide sequences for the linker peptide shown in bold font.

FIG. 5 shows the amino acid sequence (FIG. 5A, SEQ ID NO:7) and nucleotide coding sequence (FIG. 5B, SEQ ID NO:8) for the proteolytically cleavable albumin portion of another exemplary FVII fusion protein expressed by an exemplary engineered human RPE cell of the disclosure, with the amino acid and nucleotide sequences for the linker peptide shown in bold font.

FIG. 6 illustrates an exemplary PiggyBac transposon expression vector useful for generating engineered RPE cells expressing a FVII protein described herein, with FIG. 6A showing a vector map and FIG. 6B showing the nucleotide sequence of the vector (SEQ ID NO:9), with the promoter sequence underlined (SEQ ID NO: 10).

FIG. 7 shows the nucleotide sequence for the Cbh promoter (SEQ ID NO:21).

FIG. 8 illustrates an exemplary two-compartment hydrogel capsule of the disclosure, with lines indicating: engineered RPE cells encapsulated in a first, inner compartment formed from a mixture of a hydrogel forming polymer and a hydrogel-forming polymer covalently attached to a cell binding peptide; a second compartment; and an afibrotic compound disposed both within the second compartment and on the surface of the capsule. FIG. 8 discloses “GRGDSP” as SEQ ID NO: 49.

FIG. 9 is a bar graph showing the amount of FVII protein (quantified as picogram/cell/day (y axis)) secreted in vitro by exemplary RPE cells engineered with one of 8 different FVII expression constructs described herein.

FIG. 10 is a bar graph depicting the plasma levels of FVII protein in nude mice (2 or 3 mice per group) at 13 days following implantation of exemplary 2-compartment hydrogel capsules containing RPE cells engineered with one of the FVII expression constructs described herein. FIG. 10 discloses “GRGDSP” as SEQ ID NO: 49.

FIG. 11 is a bar graph depicting the plasma levels of FVII protein in nude mice (4 mice per group) at 6 days following implantation of exemplary 2-compartment hydrogel capsules containing RPE cells engineered using one of two different FVII expression vectors described herein.

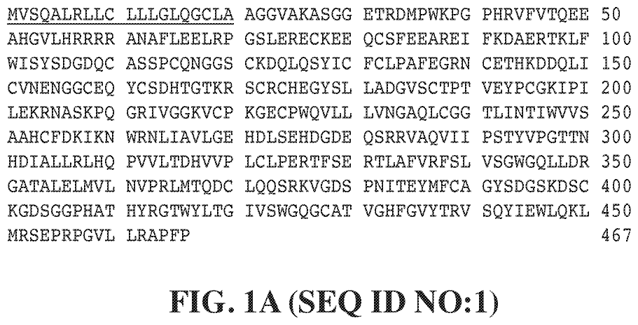

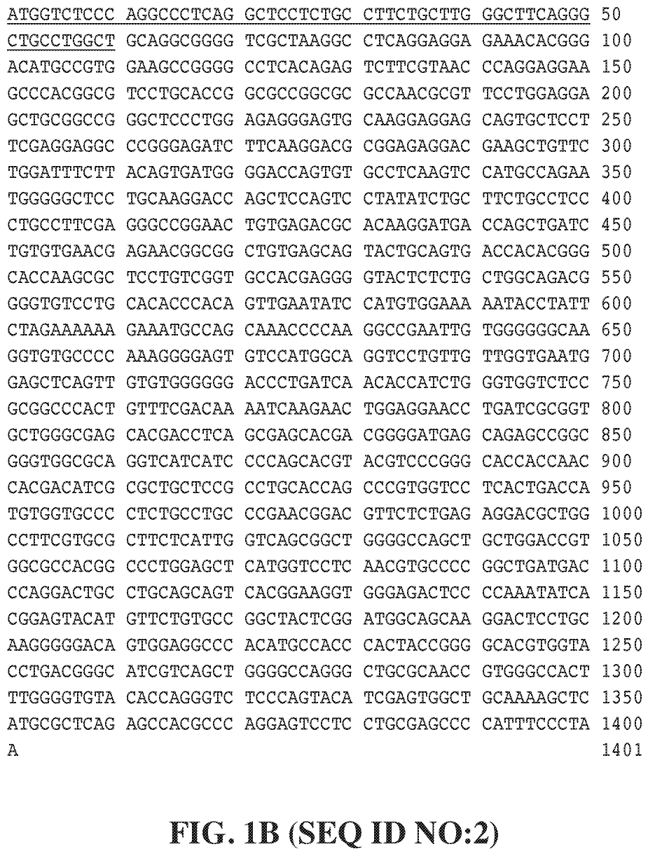

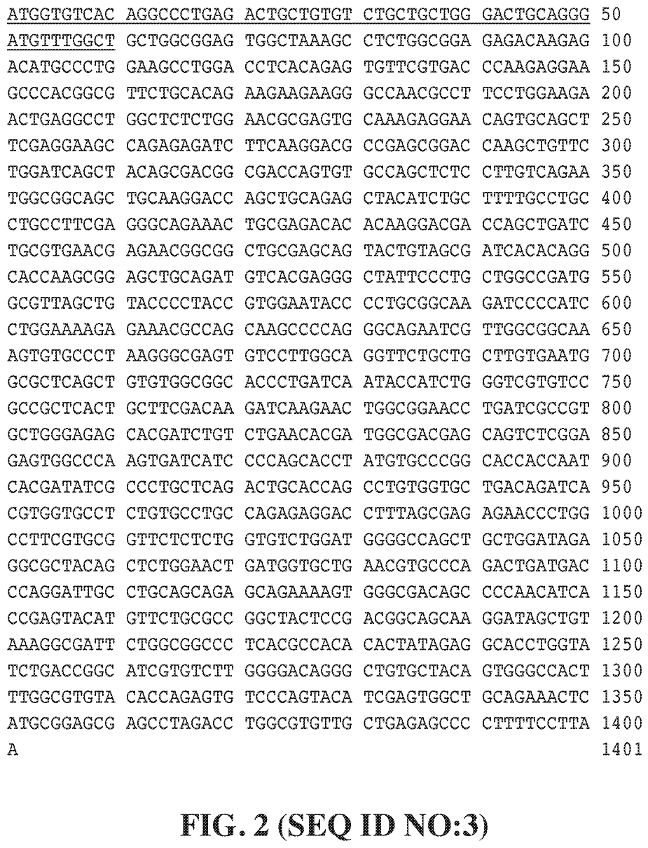

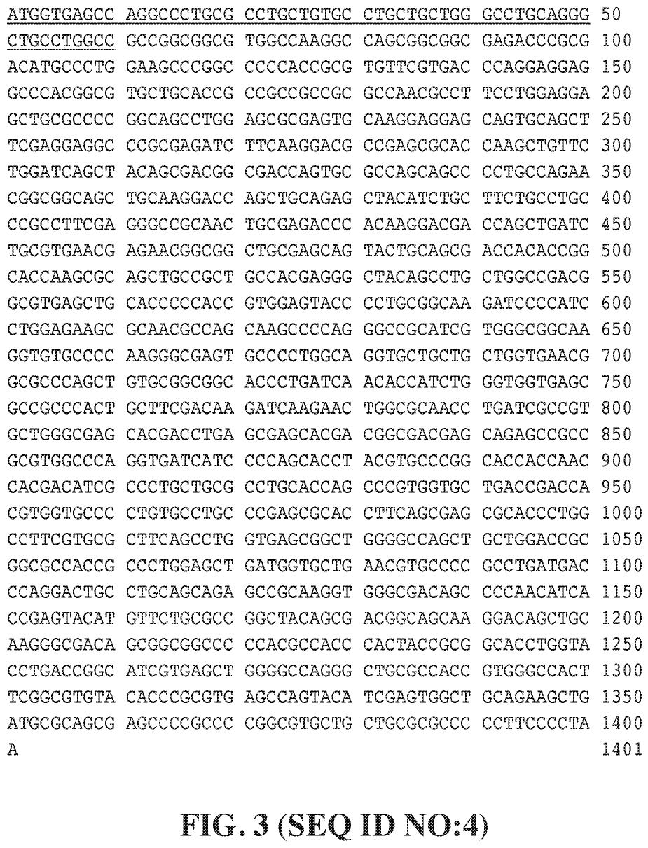

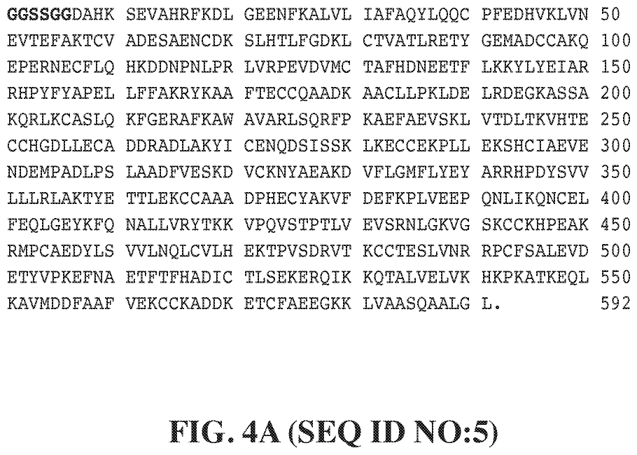

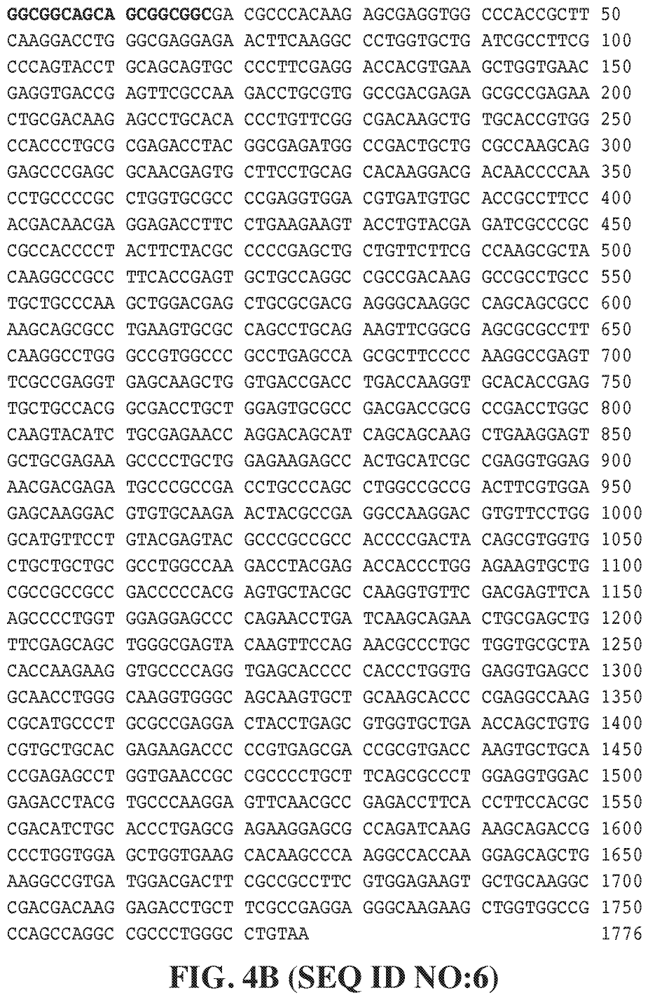

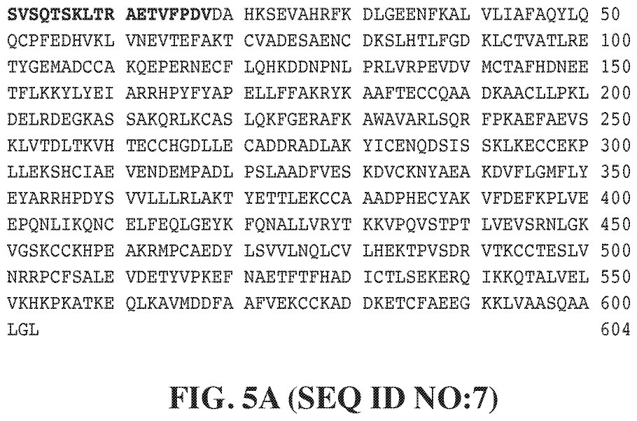

### DETAILED DESCRIPTION

The present disclosure features retinal pigment epithelial (RPE) cells engineered to express and secrete FVII, as well as compositions thereof, devices comprising such engineered RPE cells, and device preparations comprising the same. In some embodiments, the devices comprise a cell-containing compartment which includes a cell binding substance as well as the engineered RPE cells. In some embodiments, the devices are configured to mitigate the FBR when placed inside a subject, e.g., a human subject. In some embodiments, the engineered RPE cells, compositions, and devices are useful for providing FVII replacement therapy to a subject in need thereof, e.g., to patients with FVII deficiency or who are otherwise indicated for rFVIIa therapy.

### Abbreviations and Definitions

Throughout the detailed description and examples of the disclosure the following abbreviations will be used.
CBP cell-binding peptide
CBPP cell-binding polypeptide
CBP-polymer polymer covalently modified with a CBP via a linker
CBS cell-binding substance
CM-Alg chemically modified alginate
CM-LMW-Alg chemically modified, low molecular weight alginate
CM-LMW-Alg-101 low molecular weight alginate, chemically modified with Compound 101 shown in Table 3
CM-HMW-Alg chemically modified, high molecular weight alginate
CM-HMW-Alg-101 high molecular weight alginate, chemically modified with Compound 101 shown in Table 3
CM-MMW-Alg chemically modified, medium molecular weight alginate
CM-MMW-Alg-101 medium molecular weight alginate, chemically modified with Compound 101 shown in Table 3
HMW-Alg high molecular weight alginate
MMW-Alg medium molecular weight alginate
RGD-alginate an alginate covalently modified with a peptide comprising the amino acid sequence RGD (“RGD” disclosed as SEQ ID NO: 33).
RPE Retinal Pigmented Epithelial
U-Alg unmodified alginate
U-HMW-Alg unmodified high molecular weight alginate
U-LMW-Alg unmodified low molecular weight alginate
U-MMW-Alg unmodified medium molecular weight alginate
70:30 CM-Alg:U-Alg 70:30 mixture (V:V) of a chemically modified alginate and an unmodified alginate, e.g., as described in the Examples below

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise. 5“About” or “approximately”, when used herein to modify a numerically defined parameter (e.g., amount of FVII secreted by an RPE cell, a physical description of a device (e.g., hydrogel capsule) such as diameter, sphericity, number of cells encapsulated therein, the number of devices in a preparation), means that the recited numerical value is within an acceptable functional range for the defined parameter as determined by one of ordinary skill in the art, which will depend in part on how the numerical value is measured or determined, e.g., the limitations of the measurement system, including the acceptable error range for that measurement system. For example, “about” can mean a range of 20% above and below the recited numerical value. As a non-limiting example, a device defined as having a diameter of about 1.5 millimeters (mm) and encapsulating about 5 million (M) cells may have a diameter of 1.2 to 1.8 mm and may encapsulate 4 M to 6 M cells. As another non-limiting example, a preparation of about 100 devices (e.g., hydrogel capsules) includes preparations having 80 to 120 devices. In some embodiments, the term “about” means that the modified parameter may vary by as much as 15%, 10% or 5% above and below the stated numerical value for that parameter. Alternatively, particularly with respect to certain properties of the devices described herein, such as cell productivity, or density of the CBP or the afibrotic compound, the term “about” can mean within an order of magnitude above and below the recited value, e.g., within 5-fold, 4-fold, 3-fold, 2-fold or 1-fold.

“Acquire” or “acquiring”, as used herein, refers to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., using a fluorescence microscope to acquire fluorescence microscopy data.

“Administer,” “administering,” or “administration,” as used herein, refer to implanting, absorbing, ingesting, injecting, placing or otherwise introducing into a subject, an entity described herein (e.g., a device or a preparation of devices), or providing such an entity to a subject for administration.

“Afibrotic,” as used herein, means a compound or material that mitigates the foreign body response (FBR). For example, the amount of FBR in a biological tissue that is induced by implant into that tissue of a device (e.g., a hydrogel capsule) comprising an afibrotic compound (e.g., a hydrogel capsule comprising a polymer covalently modified with a compound listed in Table 3) is lower than the FBR induced by implantation of an afibrotic-null reference device, i.e., a device that lacks any afibrotic compound, but is of substantially the same composition (e.g., same CBP-polymer, same cell type(s)) and structure (e.g., size, shape, no. of compartments). In an embodiment, the degree of the FBR is assessed by the immunological response in the tissue containing the implanted device (e.g., hydrogel capsule), which may include, for example, protein adsorption, macrophages, multinucleated foreign body giant cells, fibroblasts, and angiogenesis, using assays known in the art, e.g., as described in WO 2017/075630, or using one or more of the assays/methods described Vegas, A., et al., Nature Biotechnol (supra), (e.g., subcutaneous cathepsin measurement of implanted capsules, Masson's trichrome (MT), hematoxylin or eosin staining of tissue sections, quantification of collagen density, cellular staining and confocal microscopy for macrophages (CD68 or F4/80), myofibroblasts (alpha-muscle actin, SMA) or general cellular deposition, quantification of 79 RNA sequences of known inflammation factors and immune cell markers, or FACS analysis for macrophage and neutrophil cells on retrieved devices (e.g., capsules) after 14 days in the intraperitoneal space of a suitable test subject, e.g., an immunocompetent mouse. In an embodiment, the FBR is assessed by measuring the levels in the tissue containing the implant of one or more biomarkers of immune response, e.g., cathepsin, TNF-α, IL-13, IL-6, G-CSF, GM-CSF, IL-4, CCL2, or CCL4. In some embodiments, the FBR induced by a device of the invention (e.g., a hydrogel capsule comprising an afibrotic compound disposed on its outer surface), is at least about 80%, about 85%, about 90%, about 95%, about 99%, or about 100% lower than the FBR induced by an FBR-null reference device, e.g., a device that is substantially identical to the test or claimed device except for lacking the means for mitigating the FBR (e.g., a hydrogel capsule that does not comprise an afibrotic compound but is otherwise substantially identical to the claimed capsule. In some embodiments, the FBR (e.g., level of a biomarker(s)) is measured after about 30 minutes, about 1 hour, about 6 hours, about 12 hours, about 1 day, about 2 days, about 3 days, about 4 days, about 1 week, about 2 weeks, about 1 month, about 2 months, about 3 months, about 6 months, or longer.

“Cell,” as used herein, refers to an engineered cell or a cell that is not engineered. In an embodiment, a cell is an immortalized cell, or an engineered cell derived from an immortalized cell. In an embodiment, the cell is a live cell, e.g., is viable as measured by any technique described herein or known in the art.

“Cell-binding peptide (CBP)”, as used herein, means a linear or cyclic peptide that comprises an amino acid sequence that is derived from the cell binding domain of a ligand for a cell-adhesion molecule (CAM) (e.g., that mediates cell-matrix junctions or cell-cell junctions). The CBP is less than 50, 40, 30, 25, 20, 15 or 10 amino acids in length. In an embodiment, the CBP is between 3 and 12 amino acids, 4 and 10 amino acids in length, or is 3, 4, 5, 6, 7, 8, 9 or 10 amino acids in length. The CBP amino acid sequence may be identical to the naturally occurring binding domain sequence or may be a conservatively substituted variant thereof. In an embodiment, the CAM ligand is a mammalian protein. In an embodiment, the CAM ligand is a human protein selected from the group of proteins listed in Table 1 below. In an embodiment, the CBP comprises, consists essentially of, or consists of a cell binding sequence listed in Table 1 below or a conservatively substituted variant thereof. In an embodiment, the CBP is an RGD peptide, which means the peptide comprises the amino acid sequence RGD (SEQ ID NO: 33) and optionally comprises one or more additional amino acids located at one or both of the N-terminus and C-terminus. In an embodiment, the CBP is a cyclic peptide comprising RGD, e.g., one of the cyclic RGD peptides (SEQ ID NO: 33) described in Vilaca, H. et al., Tetrahedron 70 (35):5420-5427 (2014). In an embodiment, the CBP is a linear peptide comprising RGD (SEQ ID NO: 33) and is less than 6 amino acids in length. In an embodiment, the CBP is a linear peptide that consists essentially of RGD (SEQ ID NO: 33) or RGDSP (SEQ ID NO: 48).

TABLE 1
Exemplary CAM Ligand Proteins and Cell Binding Sequences

| Protein | Cell Binding Sequence |
|---|---|
| E-cadherin | SWELYYPLRANL (SEQ ID NO: 26) |
| N-cadherin | HAVDI (SEQ ID NO: 27) |
| Collagen I | DGEA (SEQ ID NO: 28) |
| Collagen IV | FYFDLR (SEQ ID NO: 29) |
| | GFOGER (SEQ ID NO: 30) |
| | P(GPP)₅GFOGER(GPP)₅, (SEQ ID NO: 31) |
| | where O in SEQ ID NO: 30 and SEQ ID NO: 31 is 4- |
| | hydroxyproline |
| Elastin | VAPG (SEQ ID NO: 32) |
| Fibrinogen | RGD (SEQ ID NO: 33) |
| | GPR (SEQ ID NO: 34) |
| Fibronectin | RGD (SEQ ID NO: 33) |
| | KQAGDV (SEQ ID NO: 35) |
| | PHSRN (SEQ ID NO: 36) |
| | PHSRNGGGGGGRGDS (SEQ ID NO: 37) |
| | REDV (SEQ ID NO: 38) |
| Laminin | IKVAV (SEQ ID NO: 39) |
| | SRARKQAASIKVAVADR (SEQ ID NO: 40) |
| | LRE (SEQ ID NO: 41) |
| | KQLREQ (SEQ ID NO: 42) |
| | YIGSR (SEQ ID NO: 43) |
| Nidogen-1 | RGD (SEQ ID NO: 33) |
| Osteopontin | SWYGLR (SEQ ID NO: 44) |
| Tenascin C (TN-C) | AEIDGIEL (SEQ ID NO: 45) |
| Tenascin-R | RGD (SEQ ID NO: 33) |
| Tenascin-X | RGD (SEQ ID NO: 33) |
| Thrombospondin | VTCG (SEQ ID NO: 46) |
| | SVTCG (SEQ ID NO: 47) |
| Vitronectin | RGD (SEQ ID NO: 33) |
| Von Willebrand Factor | RGD (SEQ ID NO: 33) |

“CBP-polymer”, as used herein, means a polymer comprising at least one cell-binding peptide molecule covalently attached to the polymer via a linker. In an embodiment, the polymer in the CBP-polymer is not a peptide or a polypeptide. In an embodiment, the polymer in a CBP-polymer is a synthetic or naturally occurring polysaccharide, e.g., an alginate, e.g., a sodium alginate. In an embodiment, the linker is an amino acid linker (i.e., consists essentially of a single amino acid, or a peptide of several identical or different amino acids), which is joined via a peptide bond to the N-terminus or C-terminus of the CBP. In an embodiment, the C-terminus of an amino acid linker is joined to the N-terminus of the CBP and the N-terminus of the amino acid linker is joined to at least one pendant carboxyl group in the polysaccharide via an amide bond. In an embodiment, the structure of the linker-CBP is expressed as G₍₁₋₄₎-CBP, meaning that the linker has one, two, three or four glycine residues. In an embodiment, one or more of the monosaccharide moieties in a CBP-polysaccharide, e.g., a CBP-alginate) is not modified with the CBP, e.g., the unmodified moiety has a free carboxyl group or lacks a modifiable pendant carboxyl group. In an embodiment, the number of polysaccharide moieties with a covalently attached CBP is less than any of the following values: 99%, 95%, 90%, 80%, 70%, 60%, 50%, 40% 30%, 20%, 10%, 5%, 1%.

In an embodiment, the density of CBP modification in the CBP-polymer is estimated by combustion analysis for percent nitrogen, e.g., as described in the Examples below. In an embodiment, the CBP-polymer is an RGD-polymer (e.g., an RGD-alginate), which is a polymer (e.g., an alginate) covalently modified with a linker-RGD molecule (e.g., a peptide consisting essentially of GRGD (SEQ ID NO:50) or GRGDSP (SEQ ID NO:49)) and the density of modification with the linker-RGD molecule is about 0.05% nitrogen (N) to 1.00% N, about 0.10% N to about 0.75% N, about 0.20% N to about 0.50% N, or about 0.30% N to about 0.40% N, as determined using an assay described herein. In an embodiment, the conjugation density of the linker-RGD modification in an RGD-alginate (e.g., a MMW alginate covalently modified with GRGDSP (SEQ ID NO: 49)) is 0.2 to 2.0, 0.2 to 1.5, 0.2 to 1.0, 0.3 to 0.7, 0.3 to 0.6, or 0.4 to 0.6 micromoles of the linker-RGD moiety per g of the RGD-polymer in solution (e.g., saline solution) with a viscosity of 80-120 cP, as determined by any assay that is capable of quantitating the amount of a peptide conjugated to a polymer, e.g., a quantitative peptide conjugation assay described herein. Unless otherwise explicitly stated or readily apparent from the context, a specifically recited numerical concentration, concentration range, density or density range for a CBP in a CBP-polymer composition refers to the concentration of conjugated CBP molecules in the CBP-polymer composition, i.e., it does not include any residual free (e.g., unconjugated) CBP that may be present in the CBP-polymer.

“Cell-binding polypeptide (CBPP)”, as used herein, means a polypeptide of at least 50, at least 75, or at least 100 amino acids in length and comprising the amino acid sequence of a cell binding domain of a CAM ligand, or a conservatively substituted variant thereof. In an embodiment, the CAM ligand is a mammalian protein. In an embodiment, the CBPP amino acid comprises the naturally occurring amino acid sequence of a full-length CAM ligand, e.g., one of the proteins listed in Table 1, or a conservatively substituted variant thereof.

“CBP-density”, as used herein, refers to the concentration of a linker-CBP moiety in a CBP-polymer composition, e.g., an alginate modified with G₁₋₃RGD (SEQ ID NO: 53) or G₁₋₃RGDSP (SEQ ID NO: 54), unless otherwise explicitly stated herein.

“Cell-binding substance (CBS)”, as used herein, means any chemical, biological or other type of substance (e.g., a small organic compound, a peptide, a polypeptide) that is capable of mimicking at least one activity of a ligand for a cell-adhesion molecule (CAM) or other cell-surface molecule that mediates cell-matrix junctions or cell-cell junctions or other receptor-mediated signaling. In an embodiment, when present in a polymer composition encapsulating live cells, the CBS is capable of forming a transient or permanent bond or contact with one or more of the cells. In an embodiment, the CBS facilitates interactions between two or more live cells encapsulated in the polymer composition. In an embodiment, the presence of a CBS in a polymer composition encapsulating a plurality of cells (e.g., live cells) is correlated with one or both of increased cell productivity (e.g., expression of a therapeutic agent) and increased cell viability when the encapsulated cells are implanted into a test subject, e.g., a mouse. In an embodiment, the CBS is physically attached to one or more polymer molecules in the polymer composition. In an embodiment, the CBS is a cell-binding peptide or cell-binding polypeptide, as defined herein.

“Conservatively modified variants” or “conservative substitution”, as used herein, refers to a variant of a reference peptide or polypeptide that is identical to the reference molecule, except for having one or more conservative amino acid substitutions in its amino acid sequence. In an embodiment, a conservatively modified variant consists of an amino acid sequence that is at least 70%, 80%, 85%, 90%, 95%, 97%, 98% or 99% identical to the reference amino acid sequence. A conservative amino acid substitution refers to substitution of an amino acid with an amino acid having similar characteristics (e.g., charge, side-chain size, hydrophobicity/hydrophilicity, backbone conformation and rigidity, etc.) and which has minimal impact on the biological activity of the resulting substituted peptide or polypeptide. Conservative substitution tables of functionally similar amino acids are well known in the art, and exemplary substitutions grouped by functional features are set forth in Table 2 below.

TABLE 2
Exemplary conservative amino acid substitution groups.

| Feature | Conservative Amino Group |
|---|---|
| Charge/Polarity | His, Arg, Lys |
| | Asp, Glu |
| | Cys, Thr, Ser, Gly, Asn, Gln, Tyr |
| | Ala, Pro, Met, Leu, Ile, Val, Phe, Trp |
| Hydrophobicity | Asp, Glu, Asn, Gln, Arg, Lys |
| | Cys, Ser, Thr, Pro, Gly, His, Tyr |
| | Ala, Met, Ile Leu, Val, Phe, Trp |
| Structural/Surface | Asp, Glu, Asn, Aln, His, Arg, Lys |
| Exposure | Cys, Ser, Tyr, Pro, Ala, Gly, Trp, Tyr |
| | Met, Ile, Leu, Val, Phe |
| Secondary Structure | Ala, Glu, Aln, His, Lys, Met, Leu, Arg |
| Propensity | Cys, Thr, Ile, Val, Phe, Tyr, Trp |
| | Ser, Gly, Pro, Asp, Asn |
| Evolutionary | Asp, Glu |
| Conservation | His, Lys, Arg |
| | Asn, Gln |
| | Ser, Thr |
| | Leu, Ile, Val |
| | Phe, Tyr, Trp |
| | Ala, Gly |
| | Met, Cys |

“Consists essentially of”, and variations such as “consist essentially of” or “consisting essentially of” as used throughout the specification and claims, indicate the inclusion of any recited elements or group of elements, and the optional inclusion of other elements, of similar or different nature than the recited elements, that do not materially change the basic or novel properties of the specified molecule, composition, device, or method. As a non-limiting example, a cell-binding peptide or a FVII protein that consists essentially of a recited amino acid sequence may also include one or more amino acids, including substitutions in the recited amino acid sequence, of one or more amino acid residues, which do not materially affect the relevant biological activity of the cell-binding peptide or the FVII protein, respectively.

“Derived from”, as used herein with respect to a cell or cells, refers to cells obtained from tissue, cell lines, or cells, which optionally are then cultured, passaged, immortalized, differentiated and/or induced, etc. to produce the derived cell(s).

“Device”, as used herein, refers to any implantable object (e.g., a particle, a hydrogel capsule, an implant, a medical device), which contains live, engineered RPE cells capable of expressing and secreting a FVII protein following implant of the device, and has a configuration that supports the viability of the RPE cells by allowing cell nutrients to enter the device. In some embodiments, the device allows release from the device of metabolic byproducts generated by the live cells.

“Differential volume,” as used herein, refers to a volume of one compartment within a device described herein that excludes the space occupied by another compartment(s). For example, the differential volume of the second (e.g., outer) compartment in a 2-compartment device with inner and outer compartments, refers to a volume within the second compartment that excludes space occupied by the first (inner) compartment.

“Effective amount” as used herein refers to an amount of any of the following: engineered RPE cells secreting FVII, a device composition or device preparation containing such FVII-secreting cells, or a component of a device (e.g., number of engineered RPE cells in the device, amount of a CBS and/or afibrotic compound in the device) that is sufficient to elicit a desired biological response. In some embodiments, the term “effective amount” refers to the amount of a component of the device (e.g., number of cells in the device, the density of an afibrotic compound disposed on the surface and/or in a barrier compartment of the device, the density of a CBS in the cell-containing compartment. In an embodiment, the desired biological response is an increase in FVII levels in a tissue sample removed from a subject treated with (e.g., implanted with) the engineered RPE cells, a device or a device preparation containing such cells. As will be appreciated by those of ordinary skill in this art, the effective amount may vary depending on such factors as the desired biological endpoint, the pharmacokinetics of the secreted FVII, composition or device, the condition being treated, the mode of administration, and the age and health of the subject. An effective amount encompasses therapeutic and prophylactic treatment. In an embodiment, an effective amount of a compound of Formula (I) disposed on or in a device is an amount that reduces the FBR to the implanted device compared to a reference device, e.g., reduces fibrosis or amount of fibrotic tissue on or near the implanted device. In an embodiment, an effective amount of a CBS disposed with engineered RPE cells in a cell-containing compartment is an amount that enhances the viability of the cells (e.g., number of live cells) compared to a reference device and/or increases the production of FVII by the RPE cells (e.g., increased FVII levels in plasma of a subject implanted with the device) compared to a reference device. An effective amount of a device, composition or component (e.g., afibrotic compound, CBS, engineered cells) may be determined by any technique known in the art of described herein.

In an embodiment, the CBS (e.g., an alginate modified with an RGD peptide, e.g. GRGDSP-alginate (SEQ ID NO: 49)) in the cell-containing compartment is present in an amount effective to increase viability of the cells and/or increase productivity of the cells at a timepoint after the device is implanted into an immune-compromised or immune-competent animal, e.g., immune-competent mice (e.g., the C57BL/6J mouse strain available from the Jackson Laboratory, Bar Harbor, Me. USA) as compared to a CBS-null reference device, as defined below herein. In an embodiment, the increase in cell viability and/or productivity is detectable at a desired timepoint after implant, e.g., at one or more of 1 day, 3 days, 5 days, 1 week, 2 weeks, 4 weeks, 8 weeks, 12 weeks, 24 weeks, 36 weeks and 48 weeks. In an embodiment, the effective amount of the CBS results in an increase in one or both of (i) cell viability by at least 10%, 25%, 50% or 100% when measured at 1 week, 2 weeks, 4 weeks or 12 weeks after implant and (ii) increases cell productivity by at least 1.25-fold, 1.5-fold, 2-fold, 5-fold, 8-fold or 10-fold when measured at 1 week, 2 weeks, 4 weeks or 12 weeks after implant. In an embodiment, the effective amount of the CBS in the cell-containing compartment falls within a range between the minimally effective amount and a higher amount at which the cell viability and/or productivity are reduced compared to a CBS-null reference device or compared to the a device containing a maximally-effective amount, e.g., the optimal amount, of the CBS in the cell-containing compartment. In an embodiment, the amount of the CBS in the cell-containing compartment is no more than 50%, 25%, 10% or 5% above or below the optimal amount, e.g., the amount that results in the greatest increase in cell viability and/or productivity as compared to the CBS-null reference device.

The number of viable (and optionally dead) cells in a device described herein may be estimated using any technique known in the art, including an assay that differentially labels live and dead cells with two fluorescent dyes followed by detection, and optionally quantification, of labeled cells using fluorescent microscopy. Cell viability may also be evaluated by assessing other cell viability indicators, including measuring esterase activity, or quantitating the amount of ATP in the cells.

In an embodiment, the post-implant increase in cell productivity is detected by assaying for the level of the FVII protein expressed by the cells in vivo or ex vivo (e.g., cell expression after the device has been retrieved from the animal. FVII expression may be measured extracellularly but inside the device, and/or outside of the device, e.g., in a tissue sample removed from an animal (e.g., a non-human animal) treated with a device or device preparation described herein. In an embodiment, the cell productivity is expressed as the measured amount of the FVII protein or FVII activity divided by the number of administered devices (e.g., number of capsules placed in the animal) and/or by the number of administered engineered RPE cells (e.g., approximate number of cells per capsule in the administered capsule preparation). In an embodiment, the increase in cell productivity is further normalized by dividing the determined amount or activity of FVII by the time between two time points of interest, e.g., between administration and measurement, e.g., number of hours, days or weeks. In an embodiment, the increase in productivity is determined by measuring the amount and/or activity of the FVII protein in a tissue sample removed from the animal (e.g., plasma separated from a blood sample collected from the animal), dividing the measured amount and/or activity by the number of administered devices (e.g., number of implanted 2-compartment capsules), and optionally further dividing the result by the number of days between administration and tissue sample removal.

An “endogenous nucleic acid” as used herein, is a nucleic acid that occurs naturally in a subject cell.

An “endogenous polypeptide,” as used herein, is a polypeptide that occurs naturally in a subject cell.

“Engineered RPE cell,” as used herein, is an RPE cell having a non-naturally occurring alteration, and typically comprises a nucleic acid sequence (e.g., DNA or RNA) or a polypeptide not present (or present at a different level than) in an otherwise similar RPE cell under similar conditions that is not engineered (an exogenous nucleic acid sequence). In an embodiment, an engineered RPE cell comprises an exogenous nucleic acid (e.g., a vector or an altered chromosomal sequence), encoding a FVII protein. In an embodiment, an engineered RPE cell secretes a FVII protein comprising a human wild-type FVII amino acid sequence or variant thereof.

In an embodiment, the exogenous nucleic acid sequence is chromosomal (e.g., the exogenous nucleic acid sequence is an exogenous sequence disposed in endogenous chromosomal sequence) or is extra chromosomal (e.g., a non-integrated expression vector). In an embodiment, the exogenous nucleic acid sequence comprises an RNA sequence, e.g., an mRNA. In an embodiment, the exogenous nucleic acid sequence comprises a chromosomal or extra-chromosomal exogenous nucleic acid sequence that comprises a sequence which is expressed as RNA, e.g., mRNA or a regulatory RNA. In an embodiment, the exogenous nucleic acid sequence comprises a first chromosomal or extra-chromosomal exogenous nucleic acid sequence that modulates the conformation or expression of a second nucleic acid sequence, e.g., a FVII coding sequence, wherein the second amino acid sequence can be exogenous or endogenous. For example, an engineered RPE cell can comprise an exogenous nucleic acid that controls the expression of an endogenous sequence. In an embodiment, the engineered RPE cell comprises an exogenous nucleic acid sequence which comprises a codon optimized sequence that encodes FVII and achieves higher expression of FVII than a naturally occurring FVII coding sequence. The codon optimized sequence may be generated using a commercially available algorithm, e.g., GeneOptimizer (ThermoFisher Scientific), OptimumGene™ (GenScript, Piscataway, N.J. USA), GeneGPS® (ATUM, Newark, Calif. USA), or Java Codon Adapatation Tool (JCat, www.jcat.de, Grote, A. et al., Nucleic Acids Research, Vol 33, Issue suppl_2, pp. W526-W531 (2005). In an embodiment, an engineered RPE cell (e.g., engineered ARPE-19 cell) is cultured from a population of stably transfected cells, or from a monoclonal cell line.

An “exogenous nucleic acid,” as used herein, is a nucleic acid that does not occur naturally in a subject cell.

An “exogenous polypeptide,” as used herein, is a polypeptide that does not occur naturally in a subject cell, e.g., engineered cell. Reference to an amino acid position of a specific sequence means the position of said amino acid in a reference amino acid sequence, e.g., sequence of a full-length mature (after signal peptide cleavage) wild-type protein (unless otherwise stated), and does not exclude the presence of variations, e.g., deletions, insertions and/or substitutions at other positions in the reference amino acid sequence.

“Factor VII protein” or “FVII protein” as used herein, means a polypeptide that comprises the amino acid sequence of a naturally-occurring factor VII protein or variant thereof that has a FVII biological activity, e.g., promoting blood clotting, as determined by an art-recognized assay, unless otherwise specified. Naturally occurring FVII exists as a single chain zymogen, a zymogen-like two-chain polypeptide and a fully activated two-chain form (FVIIa). FVII proteins that may be produced by engineered RPE cells described herein include wild-type primate (e.g., human), porcine, canine, and murine proteins, as well as variants of such wild-type proteins, including fragments, mutants, variants with one or more amino acid substitutions and/or deletions. Human FVII is expressed with a leader sequence, and the mature circulating single-chain zymogen contains 406 amino acids. The conversion of human factor VII to activated factor VIIa is due to serine protease cleavage of the bond between Arg152 and Ile153. Human FVIIa consists of a 20-kD 152-residue light chain with gamma-carboxyglutamic acid residues, and a 30-kD 254-residue heavy chain, which contains the catalytic domain; the light and heavy chains are held together by a disulfide bond. In some embodiments, reference to FVII includes single-chain and two-chain forms thereof, including zymogen-like and FVIIa. In some embodiments, a variant FVII protein is capable of being activated to the fully activated two-chain form (Factor VIIa) that has at least 50%, 75%, 90% or more (including >100%) of the activity of wild-type Factor VIIa. Variants of FVII and FVIIa are known, e.g., marzeptacog alfa (activated) (MarzAA) and the variants described in European Patent No. 1373493, U.S. Pat. Nos. 7,771,996, 9,476,037 and US published application No. US20080058255.

“FVII deficiency”, “F7 deficiency”, “Alexander's Disease”, “hypoproconvertinemia”, “proconvertin deficiency”, “prothrombin conversion accelerator deficiency”, and “serum prothrombin conversion accelerator deficiency” can be used interchangeably and refer to a congenital or acquired condition characterized by lower than normal FVII levels and/or less than normal FVII activity. Symptoms of FVII deficiency can vary from mild to severe, depending on the levels of functional FVII. Mild symptoms can include bruising and soft tissue bleeding, longer bleeding time from wounds or dental extractions, bleeding in joints, nosebleeds, bleeding gums, heavy menstrual periods. Patients with more severe FVII deficiency can experience destruction of joint cartilage from bleeding episodes and bleeding in the intestines, stomach, muscles or head. Babies are often diagnosed with FVII deficiency within the first 6 months of life, after sustaining a bleed in the central nervous system, such as an intracranial hemorrhage, or gastrointestinal tract. A diagnosis of factor VII deficiency is based upon identification of characteristic symptoms, a detailed patient history, a thorough clinical evaluation and coagulation tests that measure how long it takes the blood to clot, i.e., the activated partial thromboplastin time (aPTT) test and prothrombin time (PT) test. Individuals with FVII deficiency has a normal aPTT and a prolonged PT. Additional tests to confirm a FVII diagnosis include a FVII assay to measure FVII activity in the blood.

“FVII deficiency patient” as used herein, refers to an individual who has been diagnosed with or suspected of having FVII deficiency. In an embodiment, a FVII deficient patient has a mutated F7 gene. The human F7 gene contains 9 exons and spans about 12.8 kb on chromosome 13q34.

“Polymer composition”, as used herein, is a composition (e.g., a solution, mixture) comprising one or more polymers. As a class, “polymers” includes homopolymers, heteropolymers, co-polymers, block polymers, block co-polymers and can be both natural and synthetic. Homopolymers contain one type of building block, or monomer, whereas co-polymers contain more than one type of monomer.

“Polypeptide”, as used herein, refers to a polymer comprising amino acid residues linked through peptide bonds and having at least two, and in some embodiments, at least 3, 4, 5, 10, 50, 75, 100, 150 or 200 amino acid residues.

“Prevention,” “prevent,” and “preventing” as used herein refers to a treatment that comprises administering or applying a FVII replacement therapy, e.g., administering a composition of devices encapsulating engineered RPE cells (e.g., as described herein), prior to the onset of one or more symptoms of FVII deficiency to preclude the physical manifestation of the symptom(s). In some embodiments, “prevention,” “prevent,” and “preventing” require that signs or symptoms of FVII deficiency have not yet developed or have not yet been observed. In some embodiments, treatment comprises prevention and in other embodiments it does not.

“Reference device”, as used herein with respect to a claimed device (e.g., hydrogel capsule), means a device (e.g., hydrogel capsule) that (i) lacks a particular feature of the claimed device, e.g., a specified exogenous nucleotide sequence, e.g., an element that enhances mRNA or protein expression (e.g., a promoter sequence, a signal peptide sequence), an FBR-mitigating means (e.g., a barrier compartment comprising an afibrotic compound (as defined herein) or a CBS (as defined herein) (e.g., an RGD polymer), (ii) encapsulates in the cell-containing compartment about the same quantity of cells of the same cell type(s) as in the claimed device, and (iii) has a substantially similar polymer composition and structure as in the claimed device other than lacking the particular feature (e.g., the afibrotic compound or CBS). In an embodiment, the number of live, engineered RPE cells in the cell-containing compartment of a reference device is within 80% to 120%, or within 90% to 110%, of the number of live, engineered RPE cells in the cell-containing compartment of the claimed device. In an embodiment, the engineered cells in the reference and claimed devices are obtained from the same cell culture. In an embodiment, a substantially similar polymer composition means all polymers in the reference and claimed device, including the polymer component of any CBP-polymer and afibrotic polymer, as applicable, are of the same chemical and molecular weight class (e.g., an alginate with high G content and the same molecular weight range). For example, in an embodiment, the cell-containing compartment of a CBP-null reference device is formed from the unmodified version of the polymer (e.g., alginate) in the CBP-polymer used to form the cell-containing compartment of the claimed device. In some embodiments in which a claimed two-compartment hydrogel millicapsule has (i) an inner compartment formed from a CBP-polymer encapsulating the plurality of cells and (ii) an outer compartment formed from a mixture of a chemically-modified polymer (e.g., a CM-LMW-alginate as described herein) and an unmodified polymer (e.g., an U-HMW-alginate as described herein), then the outer compartments of the reference and claimed capsules are formed from the same polymer mixture, while the inner compartment of the reference capsule is formed from a suspension of cells in the same polymer mixture used for the outer compartment. In an embodiment, a substantially similar structure means the reference and claimed devices have the same number of compartments (e.g., one, two, three, etc.) and about the same size and shape.

“RPE cell” as used herein refers to a cell having one or more of the following characteristics: a) it comprises a retinal pigment epithelial cell (RPE) (e.g., cultured using the ARPE-19 cell line (ATCC® CRL-2302™)) or a cell derived or engineered therefrom, e.g., by stably transfecting cells cultured from the ARPE-19 cell line with an exogenous sequence that encodes a FVII protein or otherwise engineering such cultured ARPE-19 cells to express a FVII protein, a cell derived from a primary cell culture of RPE cells, a cell isolated directly (without long term culturing, e.g., less than 5 or 10 passages or rounds of cell division since isolation) from naturally occurring RPE cells, e.g., from a human or other mammal, a cell derived from a transformed, an immortalized, or a long term (e.g., more than 5 or 10 passages or rounds of cell division) RPE cell culture; b) a cell that has been obtained from a less differentiated cell, e.g., a cell developed, programmed, or reprogramed (e.g., in vitro) into an RPE cell or a cell that is, except for any genetic engineering, substantially similar to one or more of a naturally occurring RPE cell or a cell from a primary or long term culture of RPE cells (e.g., the cell can be derived from an IPS cell); or c) a cell that has one or more of the following properties: i) it expresses one or more of the biomarkers CRALBP, RPE-65, RLBP, BEST1, or αB-crystallin; ii) it does not express one or more of the biomarkers CRALBP, RPE-65, RLBP, BEST1, or αB-crystallin; iii) it is naturally found in the retina and forms a monolayer above the choroidal blood vessels in the Bruch's membrane; iv) it is responsible for epithelial transport, light absorption, secretion, and immune modulation in the retina; or v) it has been created synthetically, or modified from a naturally occurring cell, to have the same or substantially the same genetic content, and optionally the same or substantially the same epigenetic content, as an immortalized RPE cell line (e.g., the ARPE-19 cell line (ATCC® CRL-2302™)). In an embodiment, an RPE described herein is engineered, e.g., to have a new property, e.g., the cell is engineered to express and secrete FVII. In other embodiments, an RPE cell is not engineered.

“Saline solution” as used herein, means normal saline, i.e., water containing 0.9% NaCl, unless otherwise specified.

“Sequence identity” or “percent identical”, when used herein to refer to two nucleotide sequences or two amino acid sequences, means the two sequences are the same within a specified region, or have the same nucleotides or amino acids at a specified percentage of nucleotide or amino acid positions within the specified when the two sequences are compared and aligned for maximum correspondence over a comparison window or designated region. Sequence identity may be determined using standard techniques known in the art including, but not limited to, any of the algorithms described in US Patent Application Publication No. 2017/02334455 A1. In an embodiment, the specified percentage of identical nucleotide or amino acid positions is at least about 80%, 85%, 90%, 91%, 92%, 93%, 94%, 95%, 96%, 97%, 98%, 99% or higher.

“Spherical”, as used herein, means a device (e.g., a hydrogel capsule or other particle) having a curved surface that forms a sphere (e.g., a completely round ball) or sphere-like shape, which may have waves and undulations, e.g., on the surface. Spheres and sphere-like objects can be mathematically defined by rotation of circles, ellipses, or a combination around each of the three perpendicular axes, a, b, and c. For a sphere, the three axes are the same length. Generally, a sphere-like shape is an ellipsoid (for its averaged surface) with semi-principal axes within 10%, or 5%, or 2.5% of each other. The diameter of a sphere or sphere-like shape is the average diameter, such as the average of the semi-principal axes.

“Spheroid”, as that term is used herein to refer to a device (e.g., a hydrogel capsule or other particle), means the device has (i) a perfect or classical oblate spheroid or prolate spheroid shape or (ii) has a surface that roughly forms a spheroid, e.g., may have waves and undulations and/or may be an ellipsoid (for its averaged surface) with semi-principal axes within 100% of each other.

“Subject” as used herein refers to a human or non-human animal. In an embodiment, the subject is a human (i.e., a male or female) of any age group, e.g., a pediatric human subject (e.g., infant, child, adolescent) or adult human subject (e.g., young adult, middle-aged adult, or senior adult)). In an embodiment, the subject is a non-human animal, for example, a mammal (e.g., a mouse, a dog, a primate (e.g., a cynomolgus monkey or a rhesus monkey). In an embodiment, the subject is a commercially relevant mammal (e.g., cattle, pig, horse, sheep, goat, cat, or dog) or a bird (e.g., a commercially relevant bird such as a chicken, duck, goose, or turkey). In certain embodiments, the animal is a mammal. The animal may be a male or female and at any stage of development. A non-human animal may be a transgenic animal.

“Total volume,” as used herein, refers to a volume within one compartment of a multi-compartment device that includes the space occupied by another compartment. For example, the total volume of the second (e.g., outer) compartment of a two-compartment device refers to a volume within the second compartment that includes space occupied by the first compartment.

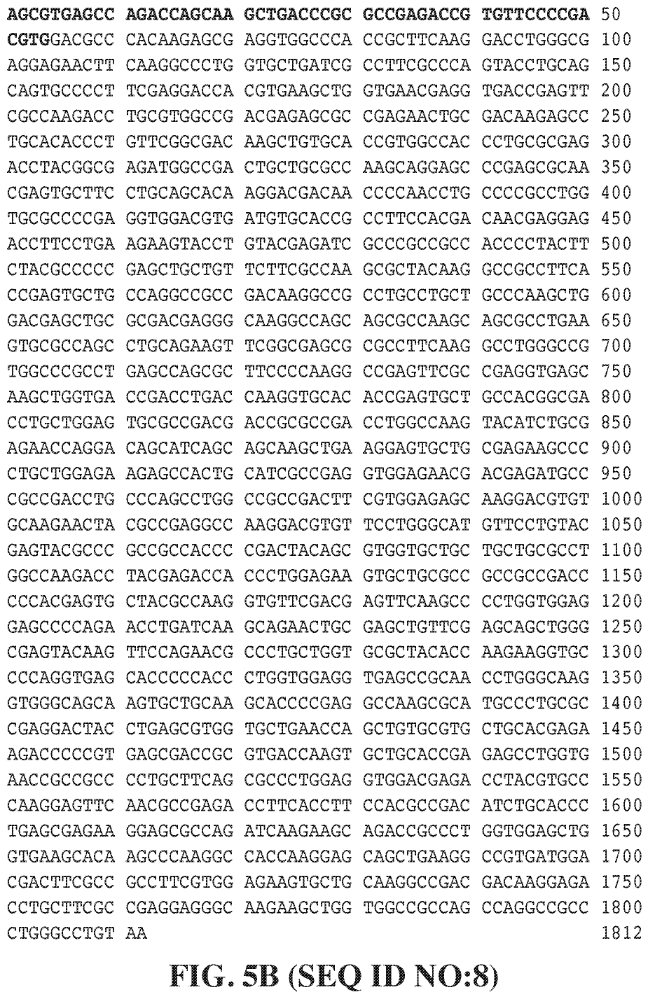

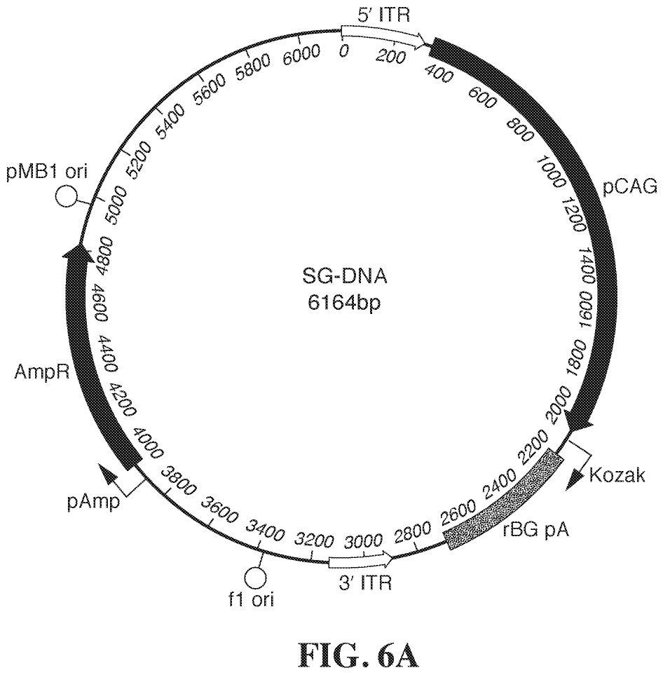

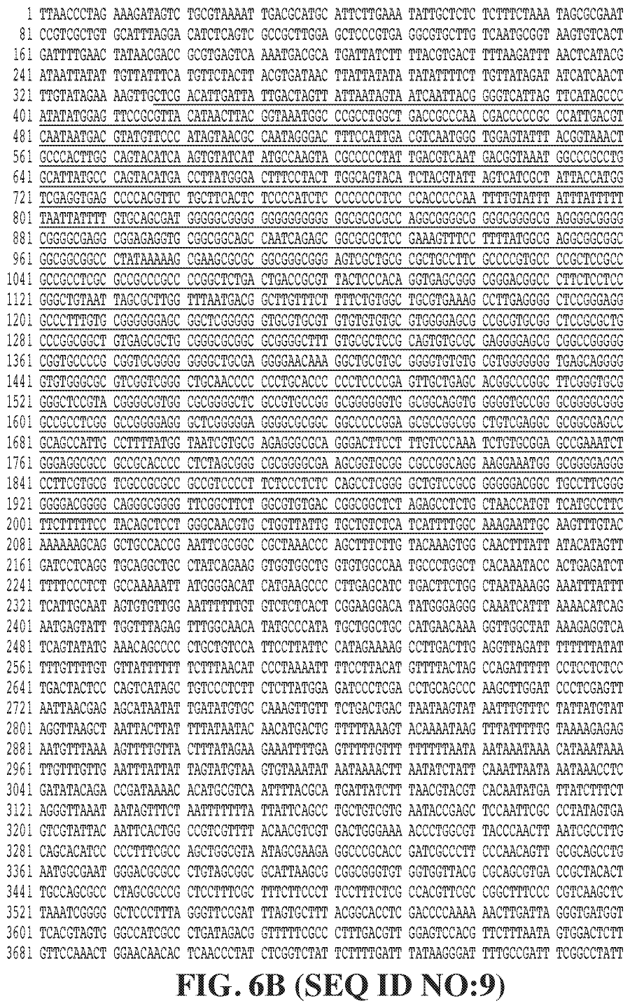

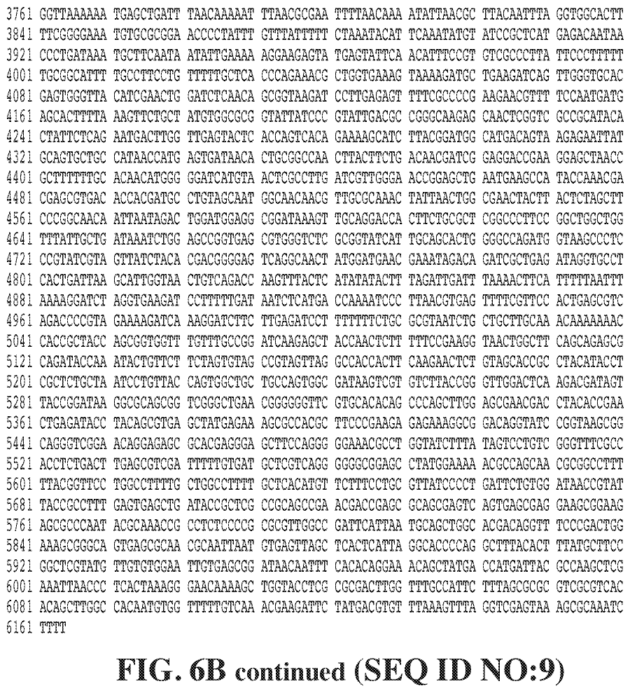

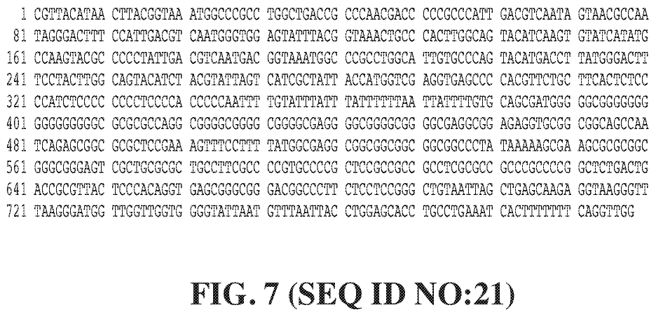

“Transcription unit” means a DNA sequence, e.g., present in an exogenous nucleic acid, that comprises at least a promoter sequence operably linked to a coding sequence, and may also comprise one or more additional elements that control or enhance transcription of the coding sequence into RNA molecules or translation of the R-NA molecules into polypeptide molecules. In some embodiments, a transcription unit also comprises polyadenylation (polyA) signal sequence and polyA site. In an embodiment, a transcription unit is present in an exogenous, extra-chromosomal expression vector, e.g., as shown in FIG. 6, or is present as an exogenous sequence integrated in a chlorosome of an engineered RPE cell described herein.

“Treatment,” “treat,” and “treating” as used herein refers to one or more of reducing, reversing, alleviating, delaying the onset of, or inhibiting the progress of one or more of a symptom, manifestation, or underlying cause, of FVII deficiency. In an embodiment, treating comprises reducing, reversing, alleviating, delaying the onset of, or inhibiting the progress of a symptom or condition associated with FVII deficiency. In an embodiment, treating comprises increasing FVII plasma levels in a subject in need thereof. In some embodiments, “treatment,” “treat,” and “treating” require that signs or symptoms associated with FVII deficiency have developed or have been observed. In other embodiments, treatment may be administered in the absence of signs or symptoms of FVII deficiency, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. In some embodiments, treatment comprises prevention and in other embodiments it does not.

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and subrange within the range. For example, “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”), 1 to 10 carbon atoms (“C₁-C₁₀ alkyl”), 1 to 8 carbon atoms (“C₁-C₈ alkyl”), 1 to 6 carbon atoms (“C₁-C₆ alkyl”), 1 to 5 carbon atoms (“C₁-C₅ alkyl”), 1 to 4 carbon atoms (“C₁-C₄alkyl”), 1 to 3 carbon atoms (“C₁-C₃ alkyl”), 1 to 2 carbon atoms (“C₁-C₂ alkyl”), or 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 12 carbon atoms (“C₂-C₁₂alkenyl”), 2 to 10 carbon atoms (“C₂-C₁₀ alkenyl”), 2 to 8 carbon atoms (“C₂-C₅ alkenyl”), 2 to 6 carbon atoms (“C₂-C₆ alkenyl”), 2 to 5 carbon atoms (“C₂-C₅ alkenyl”), 2 to 4 carbon atoms (“C₂-C₄ alkenyl”), 2 to 3 carbon atoms (“C₂-C₃ alkenyl”), or 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 12 carbon atoms (“C₂-C₁₂ alkynyl”), 2 to 10 carbon atoms (“C₂-C₁₀ alkynyl”), 2 to 8 carbon atoms (“C₂-C₈ alkynyl”), 2 to 6 carbon atoms (“C₂-C₆ alkynyl”), 2 to 5 carbon atoms (“C₂-C₅ alkynyl”), 2 to 4 carbon atoms (“C₂-C₄ alkynyl”), 2 to 3 carbon atoms (“C₂-C₃ alkynyl”), or 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) 0, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

The terms “alkylene,” “alkenylene,” “alkynylene,” or “heteroalkylene,” alone or as part of another substituent, mean, unless otherwise stated, a divalent radical derived from an alkyl, alkenyl, alkynyl, or heteroalkyl, respectively. An alkylene, alkenylene, alkynylene, or heteroalkylene group may be described as, e.g., a C₁-C₆-membered alkylene, C₂-C₆-membered alkenylene, C₂-C₆-membered alkynylene, or C₁-C₆-membered heteroalkylene, wherein the term “membered” refers to the non-hydrogen atoms within the moiety. In the case of heteroalkylene groups, heteroatoms can also occupy either or both chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, the terms “arylene” and “heteroarylene,” alone or as part of another substituent, mean a divalent radical derived from an aryl and heteroaryl, respectively.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”), 3 to 6 ring carbon atoms (“C₃-C₆ cycloalkyl”), or 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro [4.5] decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, piperazinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl or thiomorpholinyl-1,1-dioxide. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Amino” as used herein refers to the radical —NR⁷⁰R⁷¹, wherein R⁷⁰ and R⁷¹ are each independently hydrogen, C₁-C₈ alkyl, C₃-C₁₀ cycloalkyl, C₄-C₁₀ heterocyclyl, C₆-C₁₀ aryl, and C₅-C₁₀ heteroaryl. In some embodiments, amino refers to NH₂.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

Alkyl, alkenyl, alkynyl, heteroalkyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds of Formula (I) described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

Compounds of Formula (I) described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the active compounds that are prepared with relatively nontoxic acids or bases, depending on the particular substituents found on the compounds described herein. When compounds of Formula (I) used to prepare devices of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds used in the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, e.g., Berge et al, Journal of Pharmaceutical Science 66: 1-19 (1977)). Certain specific compounds used in the devices of the present disclosure (e.g., a particle, a hydrogel capsule) contain both basic and acidic functionalities that allow the compounds to be converted into either base or acid addition salts. These salts may be prepared by methods known to those skilled in the art. Other pharmaceutically acceptable carriers known to those of skill in the art are suitable for use in the present disclosure.

Devices of the present disclosure may contain a compound of Formula (I) in a prodrug form. Prodrugs are those compounds that readily undergo chemical changes under physiological conditions to provide the compounds useful to mitigate the FBR to devices of the present disclosure. Additionally, prodrugs can be converted to useful compounds of Formula (I) by chemical or biochemical methods in an ex vivo environment.

Certain compounds of Formula (I) described herein can exist in unsolvated forms as well as solvated forms, including hydrated forms. In general, the solvated forms are equivalent to unsolvated forms and are encompassed within the scope of the present disclosure. Certain compounds of Formula (I) described herein may exist in multiple crystalline or amorphous forms. In general, all physical forms are equivalent for the uses contemplated by the present disclosure and are intended to be within the scope of the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0.

The term “tautomer” as used herein refers to compounds that are interchangeable forms of a compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

The symbol “ [symbol] ” as used herein refers to a connection to an entity, e.g., a polymer (e.g., hydrogel-forming polymer such as alginate) or surface of an implantable element (e.g., a particle, device (e.g., a hydrogel capsule) or material). The connection represented by “ [symbol] ” may refer to direct attachment to the entity, e.g., a polymer or an implantable element (e.g., a device), or may refer to linkage to the entity through an attachment group. An “attachment group,” as described herein, refers to a moiety for linkage of a compound of Formula (I) to an entity (e.g., a polymer or an implantable element as described herein), and may comprise any attachment chemistry known in the art. A listing of exemplary attachment groups is outlined in Bioconjugate Techniques (3^{rd} ed, Greg T. Hermanson, Waltham, Mass.: Elsevier, Inc, 2013), which is incorporated herein by reference in its entirety. In some embodiments, an attachment group comprises alkyl, alkenyl, alkynyl, heteroalkyl, cycloalkyl, heterocyclyl, aryl, heteroaryl, —C(O)—, —OC(O)—, —N(R^{C})—, —N(R^{C})C(O)—, —C(O)N(R^{C})—, —N(R^{C})N(R^{D})—, —NCN—, —C(═N(R^{C})(R^{D}))O—, —S—, —S(O)—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, wherein each of R^{A}, R^{C}, R^{D}, R^{F}, R^{G}, x and y is independently as described herein. In some embodiments, an attachment group comprises an amine, ketone, ester, amide, alkyl, alkenyl, alkynyl, or thiol. In some embodiments, an attachment group is a cross-linker. In some embodiments, the attachment group is —C(O)(C₁-C₆-alkylene)-, wherein alkylene is substituted with R¹, and R¹ is as described herein. In some embodiments, the attachment group is —C(O)(C₁-C₆-alkylene)-, wherein alkylene is substituted with 1-2 alkyl groups (e.g., 1-2 methyl groups). In some embodiments, the attachment group is —C(O)C(CH₃)₂—. In some embodiments, the attachment group is —C(O)(methylene)-, wherein alkylene is substituted with 1-2 alkyl groups (e.g., 1-2 methyl groups). In some embodiments, the attachment group is —C(O)CH(CH₃)—. In some embodiments, the attachment group is —C(O)C(CH₃)—.

### FVII Expression Constructs

The present disclosure provides an isolated polynucleotide comprising a promoter operably linked to a nucleotide sequence encoding a human FVII precursor protein or variant thereof, e.g., a FVII fusion protein. In an embodiment, the polynucleotide is a double-stranded polynucleotide.

In an embodiment, the promoter is selected to achieve higher expression of FVII mRNA in RPE cells (e.g., ARPE-19 cells) compared to the same FVII coding sequence operably linked to the promoter in the human FVII gene. In an embodiment, the promoter consists essentially of, or consists of, SEQ ID NO: 10 or a nucleotide sequence that is substantially identical to SEQ ID NO:10, e.g., is at least 95%, 96%, 97%, 98%, 99% or more identical to SEQ ID NO:10. In an embodiment, the promoter consists of SEQ ID NO: 10.

In an embodiment, the promoter consists essentially of, or consists of, SEQ ID NO:21 or a nucleotide sequence that is substantially identical to SEQ ID NO:21, e.g., is at least 95%, 96%, 97%, 98%, 99% or more identical to SEQ ID NO:10. In an embodiment, the promoter consists of SEQ ID NO:21.

In an embodiment, the FVII precursor protein comprises the amino acid sequence from a wild-type human FVII protein, e.g., SEQ ID NO: 1, or a conservatively substituted variant thereof. In an embodiment, the conservatively substituted variant has no more than 10, 9, 8, 7, 6, 5, 4, 3, 2 or 1 conservative substitutions. In an embodiment, the FVII precursor protein consists of SEQ ID NO:1.

In an embodiment, the nucleotide sequence encoding the precursor FVII protein is codon optimized for FVII expression in mammalian cells. In an embodiment, the codon-optimized sequence is SEQ ID NO:3 or a nucleotide sequence that is at least 95%, 96%, 97%, 98%, 99% or more identical to SEQ ID NO:3. In an embodiment, the codon-optimized sequence is SEQ ID NO:4 or a nucleotide sequence that is at least 95%, 96%, 97%, 98%, 99% or more identical to SEQ ID NO:4.

In an embodiment, the nucleotide sequence encodes a FVII fusion protein. In an embodiment, the FVII fusion protein comprises an amino acid sequence for precursor human FVII or a conservatively substituted variant thereof operably linked to an amino acid sequence encoding a non-FVII polypeptide. The non-FVII polypeptide can be any protein or protein domain that confers a longer half-life or other desired property to the fusion protein, e.g., albumin, an IgG Fc, a constant domain from an IgG light chain, one, two or three constant domains of an IgG heavy chain, a nanobody, transferrin, CTP (28 amino acid C-terminal peptide (CTP) of human chorionic gonadotropin (hCG) with its 4 O-glycans), XTEN, a homo-amino acid polymer (HAP), a proline-alanine-serine (PAS), or any combination thereof.

In an embodiment, the albumin portion of an FVII-albumin fusion protein consists essentially of, or consists of, SEQ ID NO:5, SEQ ID NO:7 or a conservatively substituted variant of SEQ ID NO:5 or SEQ ID NO:7. In an embodiment, the conservatively substituted variant of SEQ ID NO:5 or SEQ ID NO:7 has no more than 10, 9, 8, 7, 6, 5, 4, 3, 2 or 1 conservative substitutions and no substitutions in the linker peptide. In an embodiment, the FVII fusion protein comprises one of the following combinations of amino acid sequences:

i) SEQ ID NO:1 and SEQ ID NO:5 (referred to herein as SEQ ID NO: 11) or

ii) SEQ ID NO:1 and SEQ ID NO:7 (referred to herein as SEQ ID NO: 12).

In an embodiment, the nucleotide sequence encoding the FVII fusion protein comprises one of the following combinations of FVII and albumin coding sequences:

i) SEQ ID NO:3 and SEQ ID NO:6 (referred to herein as SEQ ID NO: 13);

ii) SEQ ID NO:3 and SEQ ID NO:8 (referred to herein as SEQ ID NO: 14);

iii) SEQ ID NO:4 and SEQ ID NO:6 (referred to herein as SEQ ID NO: 15);

iv) SEQ ID NO:4 and SEQ ID NO:8 (referred to herein as SEQ ID NO:16);

v) SEQ ID NO:2 and SEQ ID NO:6 (referred to herein as SEQ ID NO:17); and

vi) SEQ ID NO:2 and SEQ ID NO:8 (referred to herein as SEQ ID NO:18).

In an embodiment, the isolated polynucleotide comprises a transcription unit, which further comprises a Kozak translation sequence immediately upstream of the ATG start codon in the polypeptide coding sequence. In an embodiment, the Kozak translation sequence consists essentially of, or consists of, nucleotides 2094-2099 of SEQ ID NO:9 (referred to herein as SEQ ID NO:19), a nucleotide sequence that is substantially identical to SEQ ID NO:19 (e.g., is at least 95%, 96%, 97%, 98%, 99% or more identical to SEQ ID NO:19). In an embodiment, the transcription unit further comprises a polyA sequence that consists essentially of, or consists of, nucleotides 2163-2684 of SEQ ID NO:9 (referred to herein as SEQ ID NO:20) or a nucleotide sequence that is substantially identical to SEQ ID NO:20 (e.g., is at least 95%, 96%, 97%, 98%, 99% or more identical to SEQ ID NO:20). In an embodiment, the isolated polynucleotide comprises SEQ ID NO: 10 or SEQ ID NO:21 as the promoter operably linked to any of SEQ ID NOs: 2, 3, 4, 13, 14, 15, 16, 17 or 18. In an embodiment, the isolated polynucleotide comprises a UTR sequence located between the promoter and the Kozak translation sequence. In an embodiment, the UTR sequence consists essentially of, or consists of, nucleotides 2094-2375 of SEQ ID NO:22. In an embodiment, the isolated polynucleotide comprises any of SEQ ID NOs: 2, 3, 4, 13, 14, 15, 16, 17 or 18 inserted between nucleotides 2100 and 2101 of SEQ ID NO: 9. In an embodiment, the transcription unit is located between a pair of inverted terminal repeat sequences, e.g., between a 5′ ITR (e.g., nucleotides 1 to 313 of SEQ ID NO:9) and a 3′ ITR (e.g., nucleotides 2894-3128 of SEQ ID NO:9). In an embodiment, the polynucleotide comprises SEQ ID NO:23 or SEQ ID NO:24.

In an embodiment, the isolated polynucleotide comprises two, three or more transcription units. In an embodiment, each transcription unit has a different promoter operably linked to the same or different FVII coding sequence, which is optionally fused to a non-FVII coding sequence. In an embodiment, the polynucleotide comprises two transcription units that are otherwise identical except for the promoter. In an embodiment, the polynucleotide comprises two transcription units that are otherwise identical except for the promoter and the nucleotide sequence encoding the precursor FVII protein. In an embodiment, the transcription units are located in tandem between a pair of inverted terminal repeat sequences, e.g., between a 5′ ITR and a 3′ ITR.

In an embodiment, the isolated polynucleotide comprises a combination of an upstream transcription unit comprising a promoter and an FVII coding sequence and a downstream transcription unit comprising a promoter and an FVII coding sequence, wherein the combination is selected from the group consisting of:
i) Upstream: SEQ ID NO: 10 operably linked to any one of the following SEQ ID NOS: 2, 3, 4, 13, 14, 15, 16, 17 and 18
   Downstream: SEQ ID NO:21 operably linked to any one of the following SEQ ID NOS: 2, 3, 4, 13, 14, 15, 16, 17 and 18;
ii) Upstream: SEQ ID NO:21 operably linked to any one of the following SEQ ID NOS: 2, 3, 4, 13, 14, 15, 16, 17 and 18
   Downstream: SEQ ID NO:10 operably linked to any one of the following SEQ ID NOS: 2, 3, 4, 13, 14, 15, 16, 17 and 18;
iii) Upstream: SEQ ID NO:10 operably linked to SEQ ID NO:2;
   Downstream: SEQ ID NO:21 operably linked to SEQ ID NO:3;
iv) Upstream: SEQ ID NO:10 operably linked to SEQ ID:3;
   Downstream: SEQ ID NO:21 operably linked to SEQ ID NO:2;
v) Upstream: SEQ ID NO:10 operably linked to SEQ ID:2;
   Downstream: SEQ ID NO:21 operably linked to SEQ ID NO:4;
vi) Upstream: SEQ ID NO:10 operably linked to SEQ ID:3;
   Downstream: SEQ ID NO:21 operably linked to SEQ ID NO:2;
vii) Upstream: SEQ ID NO:10 operably linked to SEQ ID:3;
   Downstream: SEQ ID NO:21 operably linked to SEQ ID NO:3.
      In an embodiment, the isolated polynucleotide comprises (a) SEQ ID NO:25 or (b) SEQ ID NO:22.

### Engineered RPE Cells

The isolated polynucleotides described above are useful to generate retinal pigment epithelial (RPE) cells or cells derived from RPE cells that are engineered to express and secrete a FVII protein. In an embodiment, an engineered (e.g., recombinant) RPE cell comprises one or more of SEQ ID NOs 2, 3, 4, 13, 14, 15, 16, 17 or 18. In an embodiment, an engineered RPE cell comprises a transcription unit described herein, which may be present in an extra-chromosomal expression vector, or integrated into one or more chromosomal sites in the cell nucleus. In an embodiment, the recombinant cell comprises two, three, four or more copies of the transcription unit that are integrated in tandem in the same genomic site in the cell nucleus.

An engineered RPE cell described herein can be derived from any of a variety of strains. Exemplary strains of RPE cells include ARPE-19 cells, ARPE-19-SEAP-2-neo cells, RPE-J cells, and hTERT RPE-1 cells. In some embodiments, the engineered cell is derived from the ARPE-19 (ATCC® CRL-2302™) cell line. In some embodiments, the engineered RPE (e.g., ARPE-19) cell is propagated from a monoclonal cell line.

In an embodiment, an engineered cell described herein expresses a biomarker, e.g., an antigen, that is characteristic of an RPE cell, e.g., a naturally occurring RPE cell. In some embodiments, the biomarker (e.g., antigen) is a protein. Exemplary biomarkers include CRALBP, RPE-65, RLBP, BEST1, or αB-crystallin. In an embodiment, an engineered cell expresses at least one of CRALBP, RPE-65, RLBP, BEST1, or αB-crystallin. In an embodiment, an engineered cell expresses at least one of CRALBP and RPE-65.

Engineered RPE cells for use in devices, compositions and methods described herein, e.g., as a plurality of engineered cells contained or encapsulated in a hydrogel capsule, may be in various stages of the cell cycle. In some embodiments, at least one engineered cell in the plurality of engineered cells is undergoing cell division. Cell division may be measured using any known method in the art, e.g., as described in DeFazio A et al (1987) J Histochem Cytochem 35:571-577 and Dolbeare F et al (1983) Proc Natl Acad Sci USA 80:5573-5577, each of which is incorporated by reference in its entirety. In an embodiment at least 1, 2, 3, 4, 5, 10, or 20% of the cells are undergoing cell division, e.g., as determined by 5-ethynyl-2′deoxyuridine (EdU) assay or 5-bromo-2′-deoxyuridine (BrdU) assay. In some embodiments, cell proliferation is visualized or quantified by microscopy (e.g., fluorescence microscopy (e.g., time-lapse or evaluation of spindle formation)) or flow cytometry. In some embodiments, none of the engineered cells in the plurality of engineered cells are undergoing cell division and are quiescent. In an embodiment, less than 1, 2, 3, 4, 5, 10, or 20% of the cells are undergoing cell division, 5-ethynyl-2′deoxyuridine (EdU) assay, 5-bromo-2′-deoxyuridine (BrdU) assay, microscopy (e.g., fluorescence microscopy (e.g., time-lapse or evaluation of spindle formation), or flow cytometry.

In an embodiment, at least 1, 2, 3, 4, 5, 10, 20, 40, or 80% of the engineered RPE cells in the plurality are viable. Cell viability may be measured using any known method in the art, e.g., as described in Riss, T. et al (2013) “Cell Viability Assays” in Assay Guidance Manual (Sittapalam, G. S. et al, eds). For example, cell viability may be measured or quantified by an ATP assay, 5-ethynyl-2′deoxyuridine (EdU) assay, 5-bromo-2′-deoxyuridine (BrdU) assay. In some embodiments, cell viability is visualized or quantified by microscopy (e.g., fluorescence microscopy (e.g., time-lapse or evaluation of spindle formation) or flow cytometry. In an embodiment, at least 1, 2, 3, 4, 5, 10, 20, 40 or 80% of the RPE cells in the plurality are viable, e.g., as determined by an ATP assay, a 5-ethynyl-2′deoxyuridine (EdU) assay, a 5-bromo-2′-deoxyuridine (BrdU) assay, microscopy (e.g., fluorescence microscopy (e.g., time-lapse or evaluation of spindle formation)), or flow cytometry.

Any of the parameters described herein may be assessed using standard techniques known to one of skill in the art, such as histology, microscopy, and various functional assays.

### Measuring FVII Activity

The activity of FVII secreted by engineered cells or device described herein may be measured by any direct or indirect FVII activity assay known in the art or described in the Examples below.

For example, FVII biological activity in a sample of a biological fluid, e.g., plasma, may be quantified by (i) measuring the amount of Factor Xa produced in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to tissue factor (TF) using an instrument based on surface plasmon resonance (Persson, FEBS Letts. 413:359-363, 1997); or (iv) measuring hydrolysis of a synthetic substrate; and/or (v) measuring generation of thrombin in a TF-independent in vitro system. In an embodiment, FVII activity is assessed by a commercially available chromogenic assay (BIOPHEN FVII, HYPHEN BioMed Neuville sur Oise, France), in which the biological sample containing FVII is mixed with thromboplastin calcium, Factor X and SXa-11 (a chromogenic substrate specific for Factor Xa.

### Devices

An engineered RPE cell described herein or a plurality of such cells may be incorporated into an implantable device for use in providing a FVII protein to a subject, e.g., to a patient with a bleeding disorder, e.g., a hemophilia patient with inhibitors for FVIII or FIX replacement therapy, a patient with FVII deficiency.

Exemplary implantable devices comprise materials such as metals, metallic alloys, ceramics, polymers, fibers, inert materials, and combinations thereof. The device (e.g., particle) can have any configuration and shape appropriate for supporting the viability and productivity of the encapsulated cells after implant into the intended target location. In some embodiments, the device is a hydrogel capsule, e.g., a millicapsule or a microcapsule (e.g., a hydrogel millicapsule or a hydrogel microcapsule). The device (e.g., capsule, particle) may comprise (and optionally is configured to release) one or more exogenous agents that are not expressed by the engineered RPE cells, and may include, e.g., a nucleic acid (e.g., an RNA or DNA molecule), a protein (e.g., a hormone, an enzyme (e.g., glucose oxidase, kinase, phosphatase, oxygenase, hydrogenase, reductase) antibody, antibody fragment, antigen, or epitope)), small molecule, lipid, drug, vaccine, or any derivative thereof, a small-molecule, an active or inactive fragment of a protein or polypeptide. In some embodiments, the device comprises at least one means for mitigating the foreign body response (FBR), for example, mitigate the FBR when the device is implanted into or onto a subject.

A device described herein may be provided as a preparation or composition for implantation or administration to a subject, i.e., a device preparation or device composition. In some embodiments, a device preparation or device composition comprises at least 2, 4, 8, 16, 32, 64 or more devices, and at least 20%, 30%, 35%, 40%, 45%, 50%, 55%, 60%, 65%, 70%, 75%, 80%, 85%, 90%, 95% or 100% of the devices in the preparation or composition have a characteristic as described herein, e.g., mean capsule diameter, or number of cells in the cell-containing compartment.

A device, device preparation, or device composition may be configured for implantation, or is implanted or disposed, into or onto any site or part of the body. In some embodiments, the implantable device or device preparation is configured for implantation into the peritoneal cavity (e.g., the lesser sac, also known as the omental bursa or bursalis omentum). A device, device preparation or device composition may be implanted in the peritoneal cavity (e.g., the omentum, e.g., the lesser sac) or disposed on a surface within the peritoneal cavity (e.g., omentum, e.g., lesser sac) via injection or catheter. Additional considerations for implantation or disposition of a device, device preparation or device composition into the omentum (e.g., the lesser sac) are provided in M. Pellicciaro et al. (2017) CellR4 5(3):e2410.

In some embodiments, the implantable device comprises at least one cell-containing compartment comprising a plurality of live cells encapsulated by a polymer composition. In an embodiment, the device contains two, three, four or more cell-containing compartments. Each cell-containing compartment comprises a plurality of live cells and the cells in at least one of the compartments are capable of expressing and secreting an FVII protein when the device is implanted into a subject.

In some embodiments, the polymer composition in the cell-containing compartment(s) comprises a polysaccharide or other hydrogel-forming polymer (e.g., alginate, hyaluronate or chondroitin). In some embodiments, the polymer is an alginate, which is a polysaccharide made up of β-D-mannuronic acid (M) and α-L-guluronic acid (G). In some embodiments, the alginate has a low molecular weight (e.g., approximate molecular weight of <75 kD) and G:M ratio ≥1.5, (ii) a medium molecular weight alginate, e.g., has approximate molecular weight of 75-150 kDa and G:M ratio ≥1.5, (iii) a high molecular weight alginate, e.g., has an approximate MW of 150 kDa-250 kDa and G:M ratio ≥1.5, (iv) or a blend of two or more of these alginates.

In some embodiments, the cell-containing compartment(s) further comprises at least one cell-binding substance (CBS), e.g., a cell-binding peptide (CBP) or cell-binding polypeptide (CBPP). In an embodiment, the CBS comprises a CBP covalently attached to polymer molecules in the polymer composition via a linker (“CBP-polymer”). In an embodiment, the polymer in the CBP-polymer is a polysaccharide (e.g., an alginate) or other hydrogel-forming polymer. Various cell-binding peptides for use in the devices of the disclosure are described herein. In an embodiment, the cell-binding peptide is 25 amino acids or less (e.g., 20, 15, 10 or less) in length and comprises the cell binding sequence of a ligand for a cell-adhesion molecule (CAM). In an embodiment, the cell-binding peptide consists essentially of a cell binding sequence shown in Table 1 herein. In an embodiment, the cell binding sequence is RGD (SEQ ID NO: 33) or RGDSP (SEQ ID NO: 48). In an embodiment, the amino terminus of the cell-binding peptide is covalently attached to the polymer via an amino acid linker. In an embodiment, the amino acid linker consists essentially of one to three glycine residues. In an embodiment, the cell-binding peptide consists essentially of RGD (SEQ ID NO: 33) or RGDSP (SEQ ID NO: 48) and the linker consists essentially of a single glycine residue.

In an embodiment, each CBP-polymer present in the cell-containing compartment (e.g., inner compartment of a two-compartment hydrogel capsule) has a cell-binding peptide density (% nitrogen as determined by combustion analysis as described in the Examples herein) of at least 0.05%, 0.1%, 0.2% or 0.3% but less than 4%, 3%, 2% or 1%. In an embodiment, the total density of a linker-CBP in a cell containing compartment is about 0.1 to about 1.0 micromoles of the CBP per g of CBP-polymer (e.g., a MMW-alginate covalently modified with GRGD (SEQ ID NO: 50) or GRGDSP (SEQ ID NO: 49)) in solution as determined by a quantitative peptide conjugation assay, e.g., an assay described herein. In an embodiment, the CBP is RGDSP (SEQ ID NO: 48), the linker is G and the polymer is an alginate with a molecular weight of 75 kDa to 150 kDa and a G:M ratio of greater than or equal to 1.5. In an embodiment, the cell-containing compartment also comprises an unmodified hydrogel-forming polymer which is the same or different than the polymer in the CBP-polymer. In an embodiment, the polymer in the CBP-polymer and the unmodified polymer is an alginate with a molecular weight of 75 kDa to 150 kDa and a G:M ratio of greater than or equal to 1.5.

In an embodiment, the quantitative peptide conjugation assay includes subjecting a sample of a CPB-polymer to acid hydrolysis to generate individual amino acids from the conjugated peptide (and any residual unconjugated peptide in the CBP-polymer), quantitating the individual amino acids, averaging the molar concentration of each amino acid, and calculating the total peptide concentration in the sample. In an embodiment, the quantitative peptide conjugation assay is performed substantially similar to the process described in the Examples herein below. In an embodiment, the quantitative peptide conjugation assay also includes subtracting the concentration of any residual unconjugated peptide in the sample from the total peptide concentration. The concentration of unconjugated peptide in a CBP-polymer composition may be determined using any suitable assay known in the art, e.g., by LC-MS as described herein below. Typically, the quantitative peptide conjugation assay is performed on a sample of a saline solution of the CBP-polymer that is used to prepare the device but may also be performed on a lyophilized sample of the CBP-polymer.

In some embodiments, the device further comprises at least one means for mitigating the foreign body response (FBR), for example, mitigate the FBR when the device is implanted into or onto a subject. Various means for mitigating the FBR of the devices are described herein, but any biological, chemical or physical element that is capable of reducing the FBR to the device compared to a reference device is contemplated herein.

For example, the means for mitigating the FBR in devices disclosed herein can comprise surrounding the cells with a semi-permeable biocompatible membrane having a pore size that is selected to allow oxygen and other molecules important to cell survival and function to move through the semi-permeable membrane while preventing immune cells from traversing through the pores. In an embodiment, the semi-permeable membrane has a molecular weight cutoff of less than 1000 kD or between 50-700 kD, 70-300 kD, or between 70-150 kD, or between 70 and 130 kD.

Another FBR-mitigating means comprises surrounding the cell-containing compartment with a barrier compartment formed from a cell-free biocompatible material, such as the core-shell microcapsules described in Ma, M et al., Adv. Healthc Mater., 2(5):667-672 (2012). Such a barrier compartment could be used with or without the semi-permeable member means. FBR-mitigating means can comprise disposing on or within the device an anti-inflammatory drug that is released from the implanted device to inhibit FBR, e.g., as described in U.S. Pat. No. 9,867,781. Other FBR-mitigating means employ a CSF-1R inhibitor that is disposed on the device surface or encapsulated within the device, as described in WO 2017/176792 and WO 2017/176804. Other FBR-mitigating means employ configuring the device in a spherical shape with a diameter of greater than 1 mm, as described in Veiseh, O., et al., Nature Materials 14:643-652 (2015). In some embodiments, the means for mitigating the FBR comprises disposing an afibrotic compound on the exterior surface of the device and/or within a barrier compartment surrounding the cell-containing compartment. Exemplary afibrotic compounds include compounds of Formula (I) described herein below. In some embodiments, the device can comprise combinations of two or more of the above FBR-mitigating means.

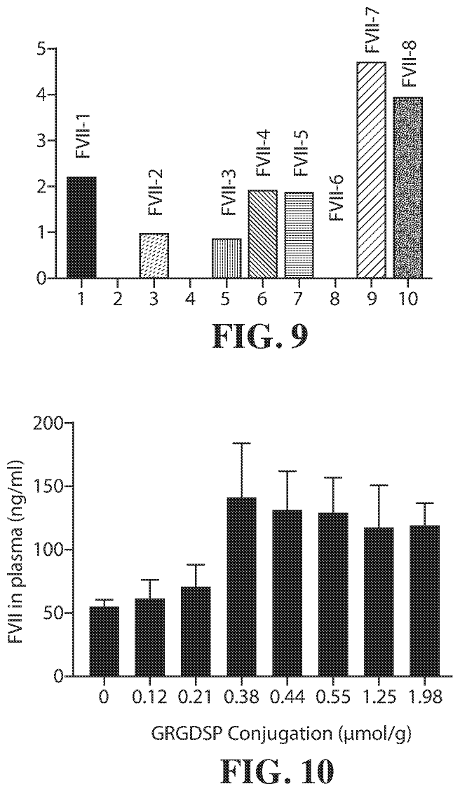

In some embodiments, the device has two hydrogel compartments, in which the inner, cell-containing compartment is completely surrounded by the second, outer (e.g., barrier) compartment. In an embodiment, the inner boundary of the second compartment forms an interface with the outer boundary of the first compartment, e.g., as illustrated in FIG. 9. In such embodiments, the thickness of the second (outer) compartment means the average distance between the outer boundary of the second compartment and the interface between the two compartments. In some embodiments, the thickness of the outer compartment is greater than about 10 nanometers (nm), preferably 100 nm or greater and can be as large as 1 millimeter (mm). For example, the thickness of the outer compartment in a hydrogel capsule device described herein may be 10 nm to 1 mm, 100 nm to 1 mm, 500 nm to 1 millimeter, 1 micrometer (μm) to 1 mm, 1 μm to 1 mm, 1 μm to 500 μm, 1 μm to 250 μm, 1 μm to 1 mm, 5 μm to 500 μm, 5 μm to 250 μm, 10 μm to 1 mm, 10 μm to 500 μm, or 10 μm to 250 μm. In some embodiments, the thickness of the outer compartment is 100 nm to 1 mm, between 1 μm and 1 mm, between 1 μm and 500 μm or between 5 μm and 1 mm. In some embodiments, the thickness of the outer compartment is between about 50 μm and about 100 μm.

In some embodiments, one or more compartments in a device comprises an afibrotic polymer, e.g., an afibrotic compound of Formula (I) covalently attached to a polymer that is the same or different than the polymer in the CBP-polymer. In an embodiment, some or all the monomers in the afibrotic polymer are modified with the same compound of Formula (I). In some embodiments, some or all the monomers in the afibrotic polymer are modified with different compounds of Formula (I). In some embodiments in which the device is a two-compartment hydrogel capsule, the afibrotic polymer is present only in the outer, barrier compartment, including its outer surface.

One or more compartments in a device may comprise an unmodified polymer that is the same or different than the polymer in the CBP-polymer and in any afibrotic polymer that is present in the device. In an embodiment, the first compartment, second compartment or all compartments in the device comprises the unmodified polymer. In some embodiments, the unmodified polymer is an unmodified alginate. In an embodiment, the unmodified alginate has a molecular weight of 150 kDa-250 kDa and a G:M ratio of 1.5.

In some embodiments, the afibrotic polymer comprises an alginate chemically modified with a Compound of Formula (I). The alginate in the afibrotic polymer may be the same or different than any unmodified alginate that is present in the device. In some embodiments, a compound of Formula (I) (e.g., Compound 101 in Table 3) is covalently attached to an alginate (e.g., an alginate with approximate MW<75 kDa, G:M ratio ≥1.5 at a conjugation density of at least 2.0% and less than 9.0% nitrogen, or 2.0% to 5% nitrogen, 3.0% to 8.0% nitrogen, 5% to 8.0% nitrogen, 4.0% to 7.0% nitrogen, 5.0% to 7.0% nitrogen, or about 6.0% to about 7.0% nitrogen or about 6.8% nitrogen as determined by combustion analysis for percent nitrogen as described in the Examples below. In an embodiment, the amount of Compound 101 produces an increase in % N (as compared with the unmodified alginate) of about 0.5% to 2% 2% to 4% N, about 4% to 6% N, about 6% to 8%, or about 8% to 10% N), where % N is determined by combustion analysis and corresponds to the amount of Compound 101 in the modified alginate.

In other embodiments, the density (e.g., concentration) of the Compound of Formula (I) (e.g., Compound 101) in the afibrotic alginate is defined as the % w/w, e.g., % of weight of amine/weight of afibrotic alginate in solution (e.g., saline) as determined by a suitable quantitative amine conjugation assay (e.g. by an assay described herein), and in certain embodiments, the density of a Compound of Formula (I) (e.g., Compound 101) is between about 1.0% w/w and about 3.0% w/w, between about 1.3% w/w and about 2.5% w/w or between about 1.5% w/w and 2.2% w/w. In an embodiment, the quantitative amine conjugation assay includes subjecting a sample of a chemically-modified polymer (e.g., an alginate modified with a Compound of Formula (I), e.g., CM-LMW-Alg-101) to acid hydrolysis to generate free amine and quantitating the total free amine in the sample. In an embodiment, the quantitative amine conjugation assay also includes subtracting the concentration of unconjugated amine (e.g., Compound of Formula (I)) in an unhydrolyzed sample from the total amine concentration. The quantitative amine conjugation assay is typically performed on a sample of a saline solution of the chemically modified alginate used to prepare the device but may also be performed on a lyophilized sample of the chemically-modified alginate. In an embodiment, the quantitative amine conjugation assay is performed substantially similar to the process described in Example 9 herein. In an embodiment, the Compound of Formula (I) is Compound 101 shown in Table 3.

The alginate in an afibrotic polymer can be chemically modified with a compound of Formula (I) using any suitable method known in the art. For example, the alginate carboxylic acid moiety can be activated for coupling to one or more amine-functionalized compounds to achieve an alginate modified with a compound of Formula (I). The alginate polymer may be dissolved in water (30 mL/gram polymer) and treated with 2-chloro-4,6-dimethoxy-1,3,5-triazine (0.5 eq) and N-methylmorpholine (1 eq). To this mixture may be added a solution of the compound of Formula (I) in acetonitrile (0.3M). The reaction may be warmed to 55° C. for 16 h, then cooled to room temperature and gently concentrated via rotary evaporation, then the residue may be dissolved, e.g., in water. The mixture may then be filtered, e.g., through a bed of cyano-modified silica gel (Silicycle) and the filter cake washed with water. The resulting solution may then be dialyzed (10,000 MWCO membrane) against water for 24 hours, e.g., replacing the water twice. The resulting solution can be concentrated, e.g., via lyophilization, to afford the desired chemically modified alginate.

### Compounds of Formula (I)

In some embodiments, the devices described herein comprise a compound of Formula (I):

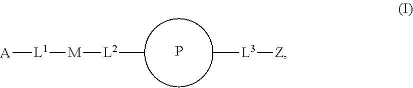

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})C(O)(C₁-C₆-alkylene)-, —N(R^{C})C(O)(C₁-C₆-alkenylene)-, —N(R^{C})N(R^{D})—, —NCN—, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and is optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is absent, cycloalkyl, heterocycyl, or heteroaryl, each of which is optionally substituted by one or more R⁴;

Z is hydrogen, alkyl, alkenyl, alkynyl, heteroalkyl, —OR^{A}, —C(O)R^{A}, —C(O)OR^{A}, —C(O)N(R^{C})(R^{D}), —N(R^{C})C(O)R^{A}, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, the compound of Formula (I) is a compound of Formula (I-a):

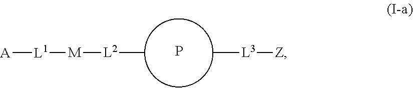

or a pharmaceutically acceptable salt thereof, wherein:

A is alkyl, alkenyl, alkynyl, heteroalkyl, cycloalkyl, heterocyclyl, aryl, heteroaryl, —O—, —C(O)O—, —C(O)—, —OC(O)—, —N(R^{C})—, —N(R^{C})C(O)—, —C(O)N(R^{C})—, —N(R^{C})N(R^{D})—, —NCN—, —N(R^{C})C(O)(C₁-C₆- alkylene)-, —N(R^{C})C(O)(C₁-C₆-alkenylene)-, —C(═N(R^{C})(R^{D}))O—, —S—, —S(O)ₓ—, —OS(O)ₓ—, —N(R^{C})S(O)ₓ—, —S(O)ₓN(R^{C})—, —P(R^{F})_{y}—, —Si(OR^{A})₂—, —Si(R^{G})(OR^{A})—, —B(OR^{A})—, or a metal, each of which is optionally linked to an attachment group (e.g., an attachment group described herein) and optionally substituted by one or more R¹;

each of L¹ and L³ is independently a bond, alkyl, or heteroalkyl, wherein each alkyl and heteroalkyl is optionally substituted by one or more R²;

L² is a bond;

M is absent, alkyl, heteroalkyl, cycloalkyl, heterocyclyl, aryl, or heteroaryl, each of which is optionally substituted by one or more R³;

P is heteroaryl optionally substituted by one or more R⁴;

Z is alkyl, alkenyl, alkynyl, heteroalkyl, cycloalkyl, heterocyclyl, aryl, or heteroaryl, each of which is optionally substituted by one or more R⁵;

each R^{A}, R^{B}, R^{C}, R^{D}, R^{E}, R^{F}, and R^{G} is independently hydrogen, alkyl, alkenyl, alkynyl, heteroalkyl, halogen, azido, cycloalkyl, heterocyclyl, aryl, or heteroaryl, wherein each alkyl, alkenyl, alkynyl, heteroalkyl, cycloalkyl, heterocyclyl, aryl, and heteroaryl is optionally substituted with one or more R⁶;

or R^{C} and R^{D}, taken together with the nitrogen atom to which they are attached, form a ring (e.g., a 5-7 membered ring), optionally substituted with one or more R⁶;

each R¹, R², R³, R⁴, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, S(O)ₓR^{E1}, —OS(O)ₓR^{E1}, —N(R^{C1})S(O)ₓR^{E1}, —S(O)ₓN(R^{C1})(R^{D1}), —P(R^{F1})_{y}, cycloalkyl, heterocyclyl, aryl, heteroaryl, wherein each alkyl, alkenyl, alkynyl, heteroalkyl, cycloalkyl, heterocyclyl, aryl, and heteroaryl is optionally substituted by one or more R⁷;

each R^{A1}, R^{B1}, R^{C1}, R^{D1}, R^{E1}, and R^{F1} is independently hydrogen, alkyl, alkenyl, alkynyl, heteroalkyl, cycloalkyl, heterocyclyl, aryl, or heteroaryl, wherein each alkyl, alkenyl, alkynyl, heteroalkyl, cycloalkyl, heterocyclyl, aryl, heteroaryl is optionally substituted by one or more R⁷;

each R⁷ is independently alkyl, alkenyl, alkynyl, heteroalkyl, halogen, cyano, oxo, hydroxyl, cycloalkyl, or heterocyclyl;

x is 1 or 2; and

y is 2, 3, or 4.

In some embodiments, for Formulas (I) or (I-a), A is alkyl, alkenyl, alkynyl, heteroalkyl, cycloalkyl, heterocyclyl, aryl, heteroaryl, —O—, —C(O)O—, —C(O)—, —OC(O)—, —N(R^{C})C(O)—, —N(R^{C})C(O)(C₁-C₆-alkylene)-, —N(R^{C})C(O)(C₁-C₆-alkenylene)-, or —N(R^{C})—. In some embodiments, A is alkyl, alkenyl, alkynyl, heteroalkyl, cycloalkyl, heterocyclyl, aryl, heteroaryl, —O—, —C(O)O—, —C(O)—, —OC(O)—, or —N(R^{C})—. In some embodiments, A is alkyl, alkenyl, alkynyl, heteroalkyl, —O—, —C(O)O—, —C(O)—, —OC(O)—, or —N(R^{C})—. In some embodiments, A is alkyl, —O—, —C(O)O—, —C(O)—, —OC(O), or —N(R^{C})—. In some embodiments, A is —N(R^{C})C(O)—, —N(R^{C})C(O)(C₁-C₆-alkylene)-, or —N(R^{C})C(O)(C₁-C₆-alkenylene)-. In some embodiments, A is —N(R^{C})—. In some embodiments, A is —N(R^{c})—, and R^{C} an R^{D} is independently hydrogen or alkyl. In some embodiments, A is —NH—. In some embodiments, A is —N(R^{C})C(O)(C₁-C₆-alkylene)-, wherein alkylene is substituted with R¹. In some embodiments, A is —N(R^{C})C(O)(C₁-C₆-alkylene)-, and R¹ is alkyl (e.g., methyl). In some embodiments, A is —NHC(O)C(CH₃)₂—. In some embodiments, A is —N(R^{C})C(O)(methylene)-, and R¹ is alkyl (e.g., methyl). In some embodiments, A is —NHC(O)CH(CH₃)—. In some embodiments, A is —NHC(O)C(CH₃)—.

In some embodiments, for Formulas (I) or (I-a), L¹ is a bond, alkyl, or heteroalkyl. In some embodiments, L¹ is a bond or alkyl. In some embodiments, L¹ is a bond. In some embodiments, L¹ is alkyl. In some embodiments, L¹ is C₁-C₆ alkyl. In some embodiments, L¹ is —CH₂—, —CH(CH₃)—, —CH₂CH₂CH₂, or —CH₂CH₂—. In some embodiments, L¹ is —CH₂— or —CH₂CH₂—.

In some embodiments, for Formulas (I) or (I-a), L³ is a bond, alkyl, or heteroalkyl. In some embodiments, L³ is a bond. In some embodiments, L³ is alkyl. In some embodiments, L³ is C₁-C₁₂alkyl. In some embodiments, L³ is C₁-C₆ alkyl. In some embodiments, L³ is —CH₂—. In some embodiments, L³ is heteroalkyl. In some embodiments, L³ is C₁-C₁₂ heteroalkyl, optionally substituted with one or more R² (e.g., oxo). In some embodiments, L³ is C₁-C₆ heteroalkyl, optionally substituted with one or more R² (e.g., oxo). In some embodiments, L³ is —C(O)OCH₂—, —CH₂(OCH₂CH₂)₂—, —CH₂(OCH₂CH₂)₃—, CH₂CH₂O—, or —CH₂O—. In some embodiments, L³ is —CH₂O—.

In some embodiments, for Formulas (I) or (I-a), M is absent, alkyl, heteroalkyl, aryl, or heteroaryl. In some embodiments, M is heteroalkyl, aryl, or heteroaryl. In some embodiments, M is absent. In some embodiments, M is alkyl (e.g., C₁-C₆ alkyl). In some embodiments, M is —CH₂—. In some embodiments, M is heteroalkyl (e.g., C₁-C₆ heteroalkyl). In some embodiments, M is (—OCH₂CH₂-)z, wherein z is an integer selected from 1 to 10. In some embodiments, z is an integer selected from 1 to 5. In some embodiments, M is —OCH₂CH₂—, (—OCH₂CH₂-)₂, (—OCH₂CH₂-)₃, (—OCH₂CH₂-)₄, or (—OCH₂CH₂-)₅. In some embodiments, M is —OCH₂CH₂—, (—OCH₂CH₂-)₂, (—OCH₂CH₂-)₃, or (—OCH₂CH₂-)₄. In some embodiments, M is (—OCH₂CH₂-)₃. In some embodiments, M is aryl. In some embodiments, M is phenyl. In some embodiments, M is unsubstituted phenyl. In some embodiments, M is

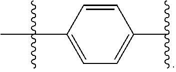 [SMILES: *c1ccc(*)cc1]

In some embodiments, M is phenyl substituted with R⁷ (e.g., 1 R⁷). In some embodiments, M is

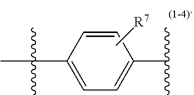

In some embodiments, R⁷ is CF₃.

In some embodiments, for Formulas (I) or (I-a), P is absent, heterocyclyl, or heteroaryl. In some embodiments, P is absent. In some embodiments, for Formulas (I) and (I-a), P is a tricyclic, bicyclic, or monocyclic heteroaryl. In some embodiments, P is a monocyclic heteroaryl. In some embodiments, P is a nitrogen-containing heteroaryl. In some embodiments, P is a monocyclic, nitrogen-containing heteroaryl. In some embodiments, P is a 5-membered heteroaryl. In some embodiments, P is a 5-membered nitrogen-containing heteroaryl. In some embodiments, P is tetrazolyl, imidazolyl, pyrazolyl, or triazolyl, pyrrolyl, oxazolyl, or thiazolyl. In some embodiments, P is tetrazolyl, imidazolyl, pyrazolyl, or triazolyl or pyrrolyl. In some embodiments, P is imidazolyl. In some embodiments, P is

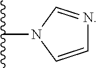 [SMILES: *n1ccnc1]

In some embodiments, P is triazolyl. In some embodiments, P is 1,2,3-triazolyl. In some embodiments, P is

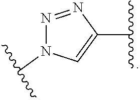 [SMILES: *c1cn(*)nn1]

In some embodiments, P is heterocyclyl. In some embodiments, P is a 5-membered heterocyclyl or a 6-membered heterocyclyl. In some embodiments, P is imidazolidinonyl. In some embodiments, P is

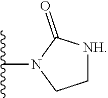 [SMILES: *N1CCNC1=O]

In some embodiments, P is thiomorpholinyl-1,1-dioxidyl.

In some embodiments, P is

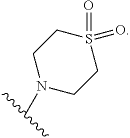 [SMILES: *N1CCS(=O)(=O)CC1]

In some embodiments, for Formulas (I) or (I-a), Z is alkyl, heteroalkyl, cycloalkyl, heterocyclyl, aryl, or heteroaryl. In some embodiments, Z is heterocyclyl. In some embodiments, Z is monocyclic or bicyclic heterocyclyl. In some embodiments, Z is an oxygen-containing heterocyclyl. In some embodiments, Z is a 4-membered heterocyclyl, 5-membered heterocyclyl, or 6-membered heterocyclyl. In some embodiments, Z is a 6-membered heterocyclyl. In some embodiments, Z is a 6-membered oxygen-containing heterocyclyl. In some embodiments, Z is tetrahydropyranyl. In some embodiments, Z is

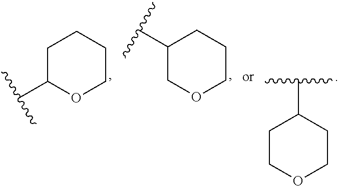

In some embodiments, Z is a 4-membered oxygen-containing heterocyclyl. In some embodiments, Z is

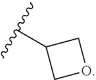 [SMILES: *C1COC1]

In some embodiments, Z is a bicyclic oxygen-containing heterocyclyl. In some embodiments, Z is phthalic anhydridyl. In some embodiments, Z is a sulfur-containing heterocyclyl. In some embodiments, Z is a 6-membered sulfur-containing heterocyclyl. n some embodiments, Z is a 6-membered heterocyclyl containing a nitrogen atom and a sulfur atom. In some embodiments, Z is thiomorpholinyl-1,1-dioxidyl. In some embodiments, Z is

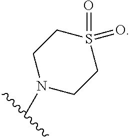 [SMILES: *N1CCS(=O)(=O)CC1]

In some embodiments, Z is a nitrogen-containing heterocyclyl. In some embodiments, Z is a 6-membered nitrogen-containing heterocyclyl. In some embodiments, Z is

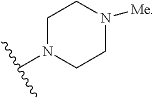 [SMILES: *N1CCN(C)CC1]

In some embodiments, Z is a bicyclic heterocyclyl. In some embodiments, Z is a bicyclic nitrogen-containing heterocyclyl, optionally substituted with one or more R⁵. In some embodiments, Z is 2-oxa-7-azaspiro[3.5]nonanyl. In some embodiments, Z is

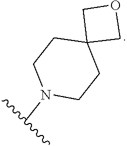 [SMILES: *N1CCC2(CC1)COC2]

In some embodiments, Z is 1-oxa-3,8-diazaspiro[4.5]decan-2-one. In some embodiments, Z is

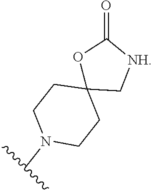 [SMILES: *N1CCC2(CC1)CNC(=O)O2]

In some embodiments, for Formulas (I) or (I-a), Z is aryl. In some embodiments, Z is monocyclic aryl. In some embodiments, Z is phenyl. In some embodiments, Z is monosubstituted phenyl (e.g., with 1 R⁵). In some embodiments, Z is monosubstituted phenyl, wherein the 1 R⁵ is a nitrogen-containing group. In some embodiments, Z is monosubstituted phenyl, wherein the 1 R⁵ is NH₂. In some embodiments, Z is monosubstituted phenyl, wherein the 1 R⁵ is an oxygen-containing group. In some embodiments, Z is monosubstituted phenyl, wherein the 1 R⁵ is an oxygen-containing heteroalkyl. In some embodiments, Z is monosubstituted phenyl, wherein the 1 R⁵ is OCH₃. In some embodiments, Z is monosubstituted phenyl, wherein the 1 R⁵ is in the ortho position. In some embodiments, Z is monosubstituted phenyl, wherein the 1 R⁵ is in the meta position. In some embodiments, Z is monosubstituted phenyl, wherein the 1 R⁵ is in the para position.

In some embodiments, for Formulas (I) or (I-a), Z is alkyl. In some embodiments, Z is C₁-C₁₂alkyl. In some embodiments, Z is C₁-C₁₀ alkyl. In some embodiments, Z is C₁-C₈ alkyl. In some embodiments, Z is C₁-C₈ alkyl substituted with 1-5 R⁵. In some embodiments, Z is C₁-C₈ alkyl substituted with 1 R⁵. In some embodiments, Z is C₁-C₈ alkyl substituted with 1 R⁵, wherein R⁵ is alkyl, heteroalkyl, halogen, oxo, —OR^{A1}, —C(O)OR^{A1}, —C(O)R^{B1}, —OC(O)R^{B1}, or —N(R^{C1})(R^{D1}). In some embodiments, Z is C₁-C₈ alkyl substituted with 1 R⁵, wherein R⁵ is —OR^{A1} or —C(O)OR^{A1}. In some embodiments, Z is C₁-C₈ alkyl substituted with 1 R⁵, wherein R⁵ is —OR^{A1} or —C(O)OH. In some embodiments, Z is —CH₃.

In some embodiments, for Formulas (I) or (I-a), Z is heteroalkyl. In some embodiments, Z is C₁-C₁₂ heteroalkyl. In some embodiments, Z is C₁-C₁₀ heteroalkyl. In some embodiments, Z is C₁-C₈ heteroalkyl. In some embodiments, Z is C₁-C₆ heteroalkyl. In some embodiments, Z is a nitrogen-containing heteroalkyl optionally substituted with one or more R⁵. In some embodiments, Z is a nitrogen and sulfur-containing heteroalkyl substituted with 1-5 R⁵. In some embodiments, Z is N-methyl-2-(methylsulfonyl)ethan-1-aminyl.

In some embodiments, Z is —OR^{A} or —C(O)OR^{A}. In some embodiments, Z is —OR^{A} (e.g., —OH or —OCH₃). In some embodiments, Z is —OCH₃. In some embodiments, Z is —C(O)OR^{A} (e.g., —C(O)OH).

In some embodiments, Z is hydrogen.

In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound of Formula (I) is a compound of Formula (I-b):

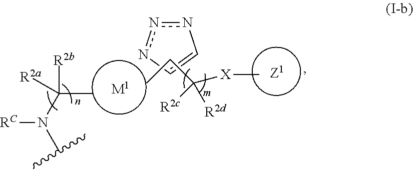

or a salt thereof, wherein Ring M¹ is cycloalkyl, heterocyclyl, aryl, or heteroaryl, each of which is optionally substituted with 1-5 R³; Ring Z¹ is cycloalkyl, heterocyclyl, aryl or heteroaryl, optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl, or each of R^{2a} and R^{2b} or R^{2c} and R^{2d} is taken together to form an oxo group; X is absent, N(R¹⁰)(R¹¹), O, or S; R^{C} is hydrogen, alkyl, alkenyl, alkynyl, heteroalkyl, cycloalkyl, heterocyclyl, aryl, or heteroaryl, wherein each of alkyl, alkenyl, alkynyl, heteroalkyl, cycloalkyl, heterocyclyl, aryl, or heteroaryl is optionally substituted with 1-6 R⁶; each R³, R⁵, and R⁶ is independently alkyl, alkenyl, alkynyl, heteroalkyl, halogen, cyano, azido, oxo, —OR^{A1}, —C(O)OR^{A1}, —C(O)R^{B1}, —OC(O)R^{B1}, —N(R^{C1})(R^{D1}), —N(R^{C1})C(O)R^{B1}, —C(O)N(R^{C1}), SR^{E1}, cycloalkyl, heterocyclyl, aryl, or heteroaryl; each of R¹⁰ and R¹¹ is independently hydrogen, alkyl, alkenyl, alkynyl, heteroalkyl, —C(O)OR^{A1}, —C(O)R^{B1}, —OC(O)R^{B1}, —C(O)N(R^{C1}), cycloalkyl, heterocyclyl, aryl, or heteroaryl; each R^{A1}, R^{B1}, R^{C1}, R^{D1}, and R^{E1} is independently hydrogen, alkyl, alkenyl, alkynyl, heteroalkyl, cycloalkyl, heterocyclyl, aryl, heteroaryl, wherein each of alkyl, alkenyl, alkynyl, heteroalkyl, cycloalkyl, heterocyclyl, aryl, heteroaryl is optionally substituted with 1-6 R⁷; each R⁷ is independently alkyl, alkenyl, alkynyl, heteroalkyl, halogen, cyano, oxo, hydroxyl, cycloalkyl, or heterocyclyl; each m and n is independently 1, 2, 3, 4, 5, or 6; and “ [symbol] ” refers to a connection to an attachment group or a polymer described herein. In some embodiments, for each R³ and R⁵, each alkyl, alkenyl, alkynyl, heteroalkyl, cycloalkyl, heterocyclyl, aryl, or heteroaryl is optionally and independently substituted with halogen, oxo, cyano, cycloalkyl, or heterocyclyl.

In some embodiments, the compound of Formula (I-b) is a compound of Formula (I-b-i):

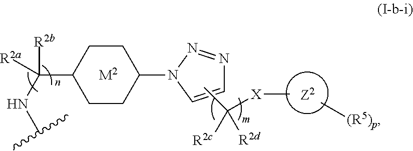

or a pharmaceutically acceptable salt thereof, wherein Ring M² is aryl or heteroaryl optionally substituted with one or more R³; Ring Z² is cycloalkyl, heterocyclyl, aryl, or heteroaryl; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; X is absent, O, or S; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; or two R⁵ are taken together to form a 5-6 membered ring fused to Ring Z²; each R^{A1} and R^{B1} is independently hydrogen, alkyl, or heteroalkyl; m and n are each independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I-b-i) is a compound of Formula (I-b-ii):

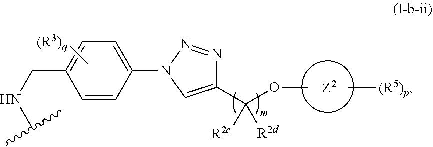

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} and taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-c):

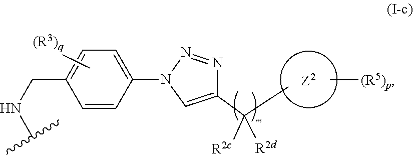

or a pharmaceutically acceptable salt thereof, wherein Ring Z² is cycloalkyl, heterocyclyl, aryl or heteroaryl; each of R^{2c} and R^{2d} is independently hydrogen, alkyl, or heteroalkyl, or R^{2c} and R^{2d} is taken together to form an oxo group; each R³ and R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; m is 1, 2, 3, 4, 5, or 6; each of p and q is independently 0, 1, 2, 3, 4, 5, or 6; and “ [symbol] ” refers to a connection to an attachment group or a polymer described herein.

In some embodiments, the compound of Formula (I) is a compound of Formula (I-d):

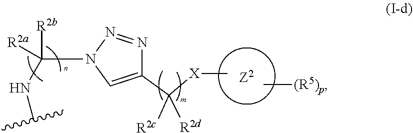

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}, wherein each alkyl and heteroalkyl is optionally substituted with halogen; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-e):

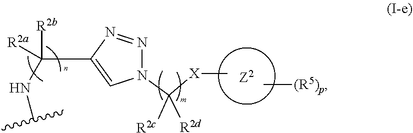

or a pharmaceutically acceptable salt thereof, wherein Ring Z² is cycloalkyl, heterocyclyl, aryl or heteroaryl; X is absent, O, or S; each of R^{2a}, R^{2b}, R^{2c}, and R^{2d} is independently hydrogen, alkyl, or heteroalkyl, or each of R^{2a} and R^{2b} or R^{2c} and R^{2d} is taken together to form an oxo group; each R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; each of m and n is independently 1, 2, 3, 4, 5, or 6; p is 0, 1, 2, 3, 4, 5, or 6; and “ [symbol] ” refers to a connection to an attachment group or a polymer described herein.

In some embodiments, the compound of Formula (I) is a compound of Formula (I-f):

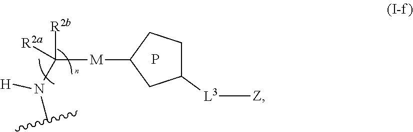

or a pharmaceutically acceptable salt thereof, wherein M is alkyl optionally substituted with one or more R³; Ring P is heteroaryl optionally substituted with one or more R⁴; L³ is alkyl or heteroalkyl optionally substituted with one or more R²; Z is alkyl, heteroalkyl, cycloalkyl, heterocyclyl, aryl, or heteroaryl, each of which is optionally substituted with one or more R⁵; each of R^{2a} and R^{2b} is independently hydrogen, alkyl, or heteroalkyl, or R^{2a} and R^{2b} is taken together to form an oxo group; each R², R³, R⁴, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (II):

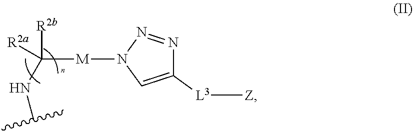

or a pharmaceutically acceptable salt thereof, wherein M is a bond, alkyl or aryl, wherein alkyl and aryl is optionally substituted with one or more R³; L³ is alkyl or heteroalkyl optionally substituted with one or more R²; Z is hydrogen, alkyl, heteroalkyl, cycloalkyl, heterocyclyl, aryl, heteroaryl or —OR^{A}, wherein alkyl, heteroalkyl, cycloalkyl, heterocyclyl, aryl, and heteroaryl is optionally substituted with one or more R⁵; R^{A} is hydrogen; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (II) is a compound of Formula (II-a):

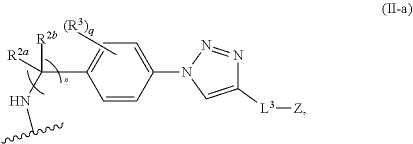

or a pharmaceutically acceptable salt thereof, wherein L³ is alkyl or heteroalkyl, each of which is optionally substituted with one or more R²; Z is hydrogen, alkyl, heteroalkyl, or —OR^{A}, wherein alkyl and heteroalkyl are optionally substituted with one or more R⁵; each of R^{2a} and R^{2b} is independently hydrogen, alkyl, or heteroalkyl, or R^{2a} and R^{2b} is taken together to form an oxo group; each R², R³, and R⁵ is independently alkyl, heteroalkyl, halogen, oxo, —OR^{A1}, —C(O)OR^{A1}, or —C(O)R^{B1}; R^{A} is hydrogen; each R^{A1} and R^{B1} is independently hydrogen, alkyl, or heteroalkyl; n is independently 1, 2, 3, 4, 5, or 6; and “ [symbol] ” refers to a connection to an attachment group or a polymer described herein.

In some embodiments, the compound of Formula (I) is a compound of Formula (III):

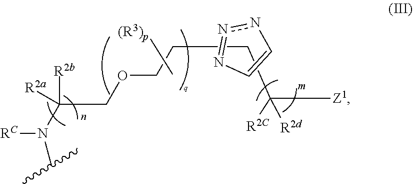

or a pharmaceutically acceptable salt thereof, wherein Z¹ is alkyl, alkenyl, alkynyl, heteroalkyl, cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, alkenyl, alkynyl, heteroalkyl, halo, cyano, nitro, amino, cycloalkyl, heterocyclyl, aryl, or heteroaryl; or R^{2a} and R^{2b} or R^{2c} and R^{2d} are taken together to form an oxo group; R^{C} is hydrogen, alkyl, alkenyl, alkynyl, or heteroalkyl, wherein each of alkyl, alkenyl, alkynyl, or heteroalkyl is optionally substituted with 1-6 R⁶; each of R³, R⁵, and R⁶ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; q is an integer from 0 to 25; and “ [symbol] ” refers to a connection to an attachment group or a polymer described herein.

In some embodiments, the compound of Formula (III) is a compound of Formula (III-a):

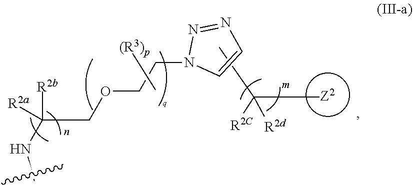

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-b):

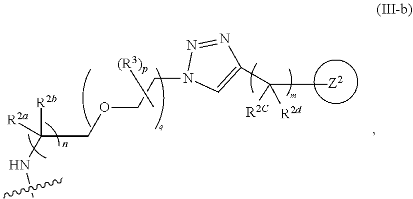

or a pharmaceutically acceptable salt thereof, wherein Ring Z² is cycloalkyl, heterocyclyl, aryl, or heteroaryl, each of which is optionally substituted with 1-5 R⁵; each of R^{2a}, R^{2b}, R^{2c}, and R^{2d} is independently hydrogen, alkyl, heteroalkyl, halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; o and p are each independently 0, 1, 2, 3, 4, or 5; q is an integer from 0 to 25; and “ [symbol] ” refers to a connection to an attachment group or a polymer described herein.

In some embodiments, the compound of Formula (III-a) is a compound of Formula (III-c):

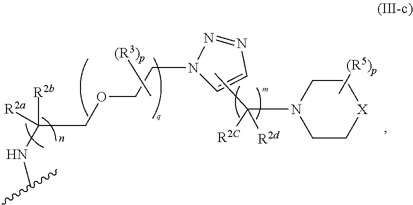

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound of Formula (III-c) is a compound of Formula (III-d):

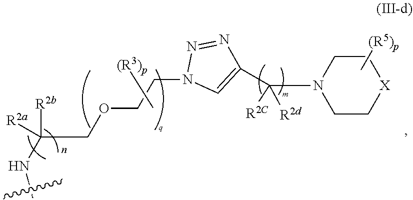

or a pharmaceutically acceptable salt thereof, wherein X is C(R′)(R″), N(R′), or S(O)ₓ; each of R′ and R″ is independently hydrogen, alkyl, halogen, or cycloalkyl; each of R^{2a}, R^{2b}, R^{2c}, and R^{2d} is independently hydrogen, alkyl, heteroalkyl, or halo; or R^{2a} and R^{2b} or R^{2c} and R^{2d} are taken together to form an oxo group; each of R³ and R⁵ is independently alkyl, heteroalkyl, halogen, oxo, —OR^{A1}, —C(O)OR^{A1}, or —C(O)R^{B1}; each R^{A1} and R^{B1} is independently hydrogen, alkyl, or heteroalkyl; m and n are each independently 1, 2, 3, 4, 5, or 6; p is 0, 1, 2, 3, 4, or 5; q is an integer from 0 to 25; x is 0, 1, or 2; and “ [symbol] ” refers to a connection to an attachment group or a polymer described herein.

In some embodiments, the compound is a compound of Formula (I). In some embodiments, L² is a bond and P and L³ are independently absent.

In some embodiments, the compound is a compound of Formula (I-a). In some embodiments of Formula (II-a), L² is a bond, P is heteroaryl, L³ is a bond, and Z is hydrogen. In some embodiments, P is heteroaryl, L³ is heteroalkyl, and Z is alkyl. In some embodiments, L² is a bond and P and L³ are independently absent. In some embodiments, L² is a bond, P is heteroaryl, L³ is a bond, and Z is hydrogen. In some embodiments, P is heteroaryl, L³ is heteroalkyl, and Z is alkyl.

In some embodiments, the compound is a compound of Formula (I-b). In some embodiments, P is absent, L¹ is —NHCH₂, L² is a bond, M is aryl (e.g., phenyl), L³ is —CH₂O, and Z is heterocyclyl (e.g., a nitrogen-containing heterocyclyl, e.g., thiomorpholinyl-1,1-dioxide). In some embodiments, the compound of Formula (I-b) is Compound 116.

In some embodiments of Formula (I-b), P is absent, L¹ is —NHCH₂, L² is a bond, M is absent, L³ is a bond, and Z is heterocyclyl (e.g., an oxygen-containing heterocyclyl, e.g., tetrahydropyranyl, tetrahydrofuranyl, oxetanyl, or oxiranyl). In some embodiments, the compound of Formula (I-b) is Compound 105.

In some embodiments, the compound is a compound of Formula (I-b-i). In some embodiments of Formula (I-b-i), each of R^{2a} and R^{2b} is independently hydrogen or CH₃, each of R^{2c} and R^{2d} is independently hydrogen, m is 1 or 2, n is 1, X is O, p is 0, M² is phenyl optionally substituted with one or more R³, R³ is —CF₃, and Z² is heterocyclyl (e.g., an oxygen-containing heterocyclyl, e.g., tetrahydropyranyl, tetrahydrofuranyl, oxetanyl, or oxiranyl). In some embodiments, the compound of Formula (I-b-i) is Compound 100, Compound 106, Compound 107, Compound 108, Compound 109, or Compound 111.

In some embodiments, the compound is a compound of Formula (I-b-ii). In some embodiments of Formula (I-b-ii), each of R^{2a}, R^{2b}, R^{2c}, and R^{2d} is independently hydrogen, q is 0, p is 0, m is 1, and Z² is heterocyclyl (e.g., an oxygen-containing heterocyclyl, e.g., tetrahydropyranyl). In some embodiments, the compound of Formula (I-b-ii) is Compound 100.

In some embodiments, the compound is a compound of Formula (I-c). In some embodiments of Formula (I-c), each of R^{2c} and R^{2d} is independently hydrogen, m is 1, p is 1, q is 0, R⁵ is —CH₃, and Z is heterocyclyl (e.g., a nitrogen-containing heterocyclyl, e.g., piperazinyl). In some embodiments, the compound of Formula (I-c) is Compound 113.

In some embodiments, the compound is a compound of Formula (I-d). In some embodiments of Formula (I-d), each of R^{2a}, R^{2b}, R^{2c}, and R^{2d} is independently hydrogen, m is 1, n is 3, X is O, p is 0, and Z is heterocyclyl (e.g., an oxygen-containing heterocyclyl, e.g., tetrahydropyranyl, tetrahydrofuranyl, oxetanyl, or oxiranyl). n some embodiments, the compound of Formula (I-d) is Compound 110 or Compound 114.

In some embodiments, the compound is a compound of Formula (I-f). In some embodiments of Formula (I-f), each of R^{2a} and R^{2b} is independently hydrogen, n is 1, M is —CH₂—, P is a nitrogen-containing heteroaryl (e.g., imidazolyl), L³ is —C(O)OCH₂—, and Z is CH₃. In some embodiments, the compound of Formula (I-f) is Compound 115.

In some embodiments, the compound is a compound of Formula (II-a). In some embodiments of Formula (II-a), each of R^{2a} and R^{2b} is independently hydrogen, n is 1, q is 0, L³ is —CH₂(OCH₂CH₂)₂, and Z is —OCH₃. In some embodiments, the compound of Formula (II-a) is Compound 112.

In some embodiments of Formula (II-a), each of R^{2a} and R^{2b} is independently hydrogen, n is 1, L³ is a bond or —CH₂, and Z is hydrogen or —OH. In some embodiments, the compound of Formula (II-a) is Compound 103 or Compound 104.

In some embodiments, the compound is a compound of Formula (III). In some embodiments of Formula (III), each of R^{2a}, R^{2b}, R^{2c}, and R^{2d} is independently hydrogen, m is 1, n is 2, q is 3, p is 0, R^{C} is hydrogen, and Z¹ is heteroalkyl optionally substituted with R⁵ (e.g., —N(CH₃)(CH₂CH₂)S(O)₂CH₃). In some embodiments, the compound of Formula (III) is Compound 120.

In some embodiments, the compound is a compound of Formula (III-b). In some embodiments of Formula (III-b), each of R^{2a}, R^{2b}, R^{2c}, and R^{2d} is independently hydrogen, m is 0, n is 2, q is 3, p is 0, and Z² is aryl (e.g., phenyl) substituted with 1 R⁵ (e.g., —NH₂). In some embodiments, the compound of Formula (III-b) is Compound 102.

In some embodiments, the compound is a compound of Formula (III-b). In some embodiments of Formula (III-b), each of R^{2a}, R^{2b}, R^{2c}, and R^{2d} is independently hydrogen, m is 1, n is 2, q is 3, p is 0, R^{C} is hydrogen, and Z² is heterocyclyl (e.g., an nitrogen-containing heterocyclyl, e.g., a nitrogen-containing spiro heterocyclyl, e.g., 2-oxa-7-azaspiro[3.5]nonanyl). In some embodiments, the compound of Formula (III-a) is Compound 121.

In some embodiments, the compound is a compound of Formula (III-d). In some embodiments of Formula (III-d), each of R^{2a}, R^{2b}, R^{2c}, and R^{2d} is independently hydrogen, m is 1, n is 2, q is 1, 2, 3, or 4, p is 0, and X is S(O)₂. In some embodiments of Formula (III-d), each of R^{2a} and R^{2b} is independently hydrogen, m is 1, n is 2, q is 1, 2, 3, or 4, p is 0, and X is S(O)₂. In some embodiments, the compound of Formula (III-d) is Compound 101, Compound 117, Compound 118, or Compound 119.

In some embodiments, the compound is a compound of Formula (I-b), (I-d), or (I-e). In some embodiments, the compound is a compound of Formula (I-b), (I-d), or (II). In some embodiments, the compound is a compound of Formula (I-b), (I-d), or (I-f). In some embodiments, the compound is a compound of Formula (I-b), (I-d), or (III).

In some embodiments, the compound of Formula (I) is not a compound disclosed in WO2012/112982, WO2012/167223, WO2014/153126, WO2016/019391, WO 2017/075630, US2012-0213708, US 2016-0030359 or US 2016-0030360.

In some embodiments, the compound of Formula (I) comprises a compound shown in Table 3, or a pharmaceutically acceptable salt thereof. In some embodiments, the exterior surface and/or one or more compartments within a device described herein comprises a small molecule compound shown in Table 3, or a pharmaceutically acceptable salt thereof.

TABLE 3
Exemplary compounds of Formula (I)

| Compound No. | Structure |
|---|---|
| 100 | 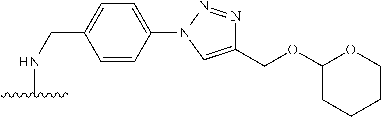 [SMILES: *NCc1ccc(-n2cc(COC3CCCCO3)nn2)cc1] |
| 101 | 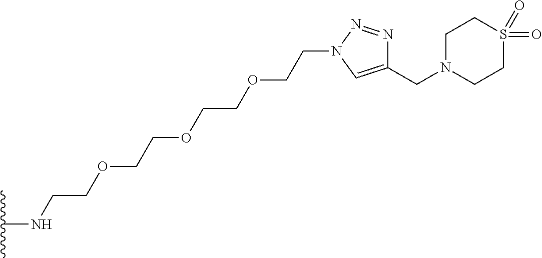 [SMILES: *NCCOCCOCCOCCn1cc(CN2CCS(=O)(=O)CC2)nn1] |
| 102 | 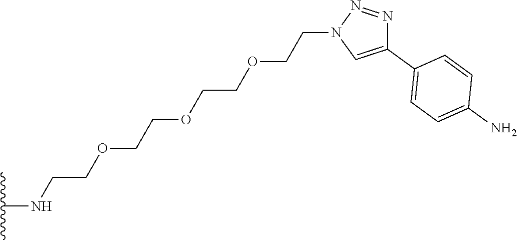 [SMILES: *NCCOCCOCCOCCn1cc(-c2ccc(N)cc2)nn1] |
| 103 | 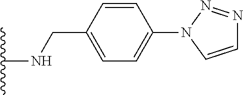 [SMILES: *NCc1ccc(-n2ccnn2)cc1] |
| 104 | 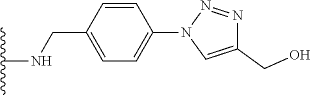 [SMILES: *NCc1ccc(-n2cc(CO)nn2)cc1] |
| 105 | 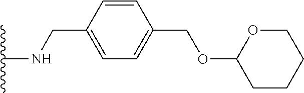 [SMILES: *NCc1ccc(COC2CCCCO2)cc1] |
| 106 | 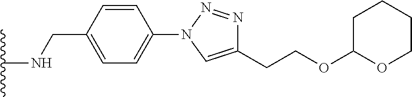 [SMILES: *NCc1ccc(-n2cc(CCOC3CCCCO3)nn2)cc1] |
| 107 | 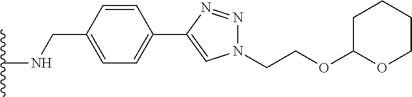 [SMILES: *NCc1ccc(-c2cn(CCOC3CCCCO3)nn2)cc1] |
| 108 | 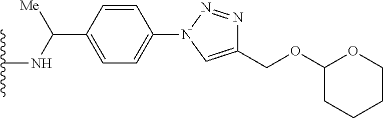 [SMILES: *NC(C)c1ccc(-n2cc(COC3CCCCO3)nn2)cc1] |
| 109 | 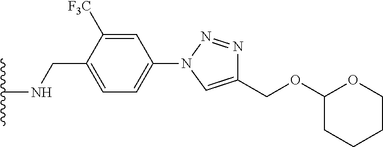 [SMILES: *NCc1ccc(-n2cc(COC3CCCCO3)nn2)cc1C(F)(F)F] |
| 110 | 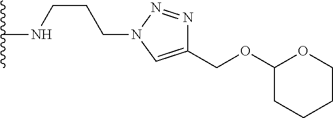 [SMILES: *NCCCn1cc(COC2CCCCO2)nn1] |
| 111 | 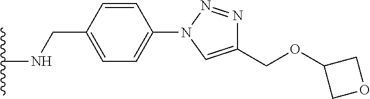 [SMILES: *NCc1ccc(-n2cc(COC3COC3)nn2)cc1] |
| 112 | 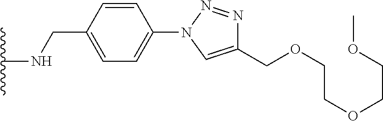 [SMILES: *NCc1ccc(-n2cc(COCCOCCOC)nn2)cc1] |
| 113 | 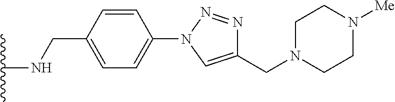 [SMILES: *NCc1ccc(-n2cc(CN3CCN(C)CC3)nn2)cc1] |
| 114 | 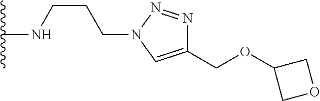 [SMILES: *NCCCn1cc(COC2COC2)nn1] |
| 115 | 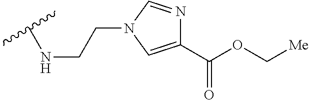 [SMILES: *NCCn1cnc(C(=O)OCC)c1] |
| 116 | 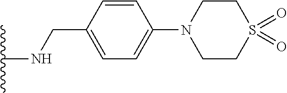 [SMILES: *NCc1ccc(N2CCS(=O)(=O)CC2)cc1] |
| 117 | 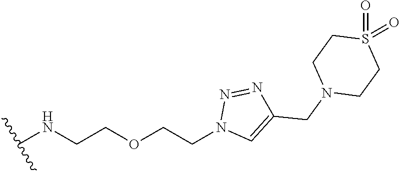 [SMILES: *NCCOCCn1cc(CN2CCS(=O)(=O)CC2)nn1] |
| 118 | 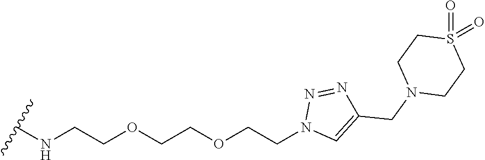 [SMILES: *NCCOCCOCCn1cc(CN2CCS(=O)(=O)CC2)nn1] |
| 119 | 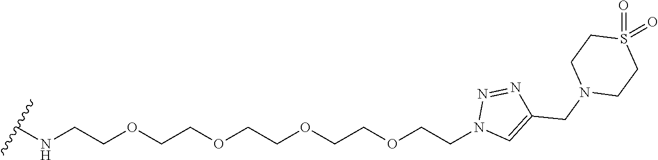 [SMILES: *NCCOCCOCCOCCOCCn1cc(CN2CCS(=O)(=O)CC2)nn1] |
| 120 | 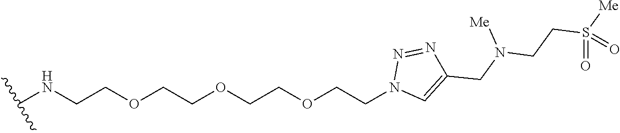 [SMILES: *NCCOCCOCCOCCn1cc(CN(C)CCS(C)(=O)=O)nn1] |
| 121 | 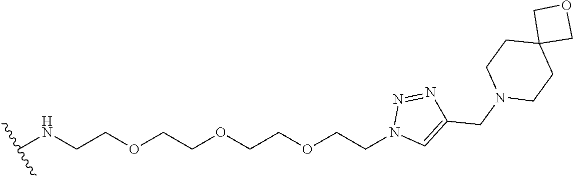 [SMILES: *NCCOCCOCCOCCn1cc(CN2CCC3(CC2)COC3)nn1] |

In some embodiments, the compound is a compound of Formula (I) (e.g., Formulas (I-a), pharmaceutically acceptable salt thereof, and is selected from:

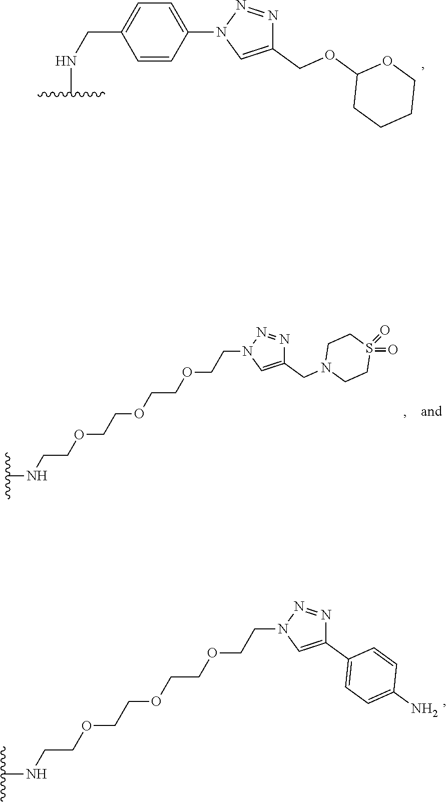 [SMILES: *NCc1ccc(-n2cc(COC3CCCCO3)nn2)cc1 ; *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of any of said compounds.

In some embodiments, the device described herein comprises the compound of

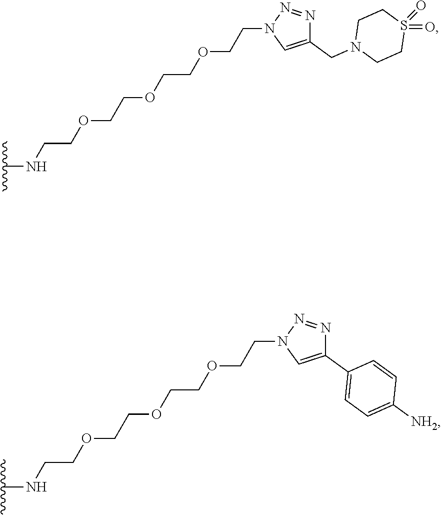 [SMILES: *NCCOCCOCCOCCn1cc(CN2CCS(=O)(=O)CC2)nn1 ; *NCCOCCOCCOCCn1cc(-c2ccc(N)cc2)nn1]

or a pharmaceutically acceptable salt of either compound.

In an embodiment, a device described herein comprises a compound of Formula (I) (e.g., a compound shown in Table 3) covalently bound to an alginate polymer. In an embodiment, a particle described herein comprises a compound of Formula (I) (e.g., a compound shown in Table 3, e.g., Compound 101) covalently bound to one or more guluronic acid and/or mannuronic acid monomers in an alginate polymer, e.g., by an amide bond.

In some embodiments, a compound of Formula (I) (e.g., Compound 101 in Table 3) is covalently attached to an alginate (e.g., an alginate with approximate MW<75 kDa, G:M ratio ≥1.5) at a conjugation density of at least 2.0% and less than 9.0% nitrogen, or 2.0% to 5% nitrogen, 3.0% to 8.0% nitrogen, 5% to 8.0% nitrogen, 4.0% to 7.0% nitrogen, 5.0% to 7.0% nitrogen, or 6.0% to 7.0% nitrogen or about 6.8% nitrogen as determined by combustion analysis for percent nitrogen as described in the Examples below.

### Methods of Treatment

Described herein are methods for preventing or treating a bleeding disorder in a subject through administration or implantation of a plurality of engineered RPE cells that are capable of expressing and secreting a FVII protein as described herein. In an embodiment, the plurality of RPE cells are contained in an implantable device described herein. In some embodiments, the methods described herein directly or indirectly reduce or alleviate at least one symptom of the bleeding disorder or prevent or slow the onset of the disorder. In an embodiment, the method comprises administering (e.g., implanting) an effective amount of a composition of two-compartment alginate hydrogel capsules which comprise in the inner compartment engineered RPE cells and a cell-binding polymer described herein and comprise a Compound of Formula (I), e.g., Compound 101, on the outer capsule surface and optionally within the outer compartment.

### Enumerated Exemplary Embodiments

1. An isolated polynucleotide comprising a coding sequence for a factor VII (FVII) protein, wherein the polynucleotide has at least one or more of the following features:
(a) a promoter operably linked to the coding sequence, wherein the promoter consists essentially of, or consists of, a nucleotide sequence that is identical to, or substantially identical to, SEQ ID NO:10 or SEQ ID NO:21;
(b) the coding sequence comprises a precursor FVII coding sequence selected from the group consisting of:
   (i) SEQ ID NO:3,
   (ii) a nucleotide sequence that is at least 95%, 96%, 97%, 98%, 99% or more identical to SEQ ID NO:3,
   (iii) SEQ ID NO:4, and
   (iv) a nucleotide sequence that is at least 95%, 96%, 97%, 98%, 99% or more identical to SEQ ID NO:4.
      2. The isolated polynucleotide of embodiment 1, wherein the precursor FVII coding sequence comprises SEQ ID NO:3 or SEQ ID NO:4.
      3. The isolated polynucleotide of embodiment 2, which comprises SEQ ID NO: 10 as a promoter operably linked to the precursor FVII coding sequence.
      4. The isolated polynucleotide of any one of embodiments 1 to 3, wherein the FVII protein is an FVII fusion protein.
      5. The isolated polynucleotide of embodiment 4, wherein the FVII fusion protein comprises SEQ ID NO:11 or SEQ ID NO:12.
      6. The isolated polynucleotide of embodiment 5, wherein the coding sequence comprises, consists essentially of or consists of SEQ ID NO:13, SEQ ID NO:14, SEQ ID NO:15, SEQ ID NO:16, SEQ ID NO:17 or SEQ ID NO:18.
      7. An isolated polynucleotide comprising an upstream transcription unit and a downstream transcription unit, wherein the upstream transcription unit comprises SEQ ID NO:10 operably linked to SEQ ID NO:3 and the downstream transcription unit comprises SEQ ID NO:21 operably linked to SEQ ID NO:3.
      8. The isolated polynucleotide of any one of the above embodiments, which is provided as an isolated double-stranded DNA molecule e.g., an expression vector.
      9. A plurality of engineered RPE cells capable of expressing and secreting a FVII protein (e.g., a human FVII protein or variant thereof), wherein each cell in the plurality comprises an exogenous nucleotide sequence comprising a coding sequence for the FVII protein, wherein the exogenous nucleotide sequence has at least one or more of the following features:
   (a) a promoter operably linked to the coding sequence, wherein the promoter consists essentially of, or consists of, a nucleotide sequence that is identical to, or substantially identical to, SEQ ID NO: 10 or SEQ ID NO:21;
   (b) the coding sequence comprises a precursor FVII coding sequence selected from the group consisting of:
      (i) SEQ ID NO:3,
      (ii) a nucleotide sequence that is at least 95%, 96%, 97%, 98%, 99% or more identical to SEQ ID NO:3,
      (iii) SEQ ID NO:4, and
      (iv) a nucleotide sequence that is at least 95%, 96%, 97%, 98%, 99% or more identical to SEQ ID NO:4.
         10. The plurality of engineered RPE cells of embodiment 9, wherein the precursor FVII coding sequence comprises SEQ ID NO:3 or SEQ ID NO:4.
         11. The plurality of engineered RPE cells of embodiment 9 or 10, wherein the exogenous nucleotide sequence comprises at least one transcription unit comprising SEQ ID NO:10 or SEQ ID NO:21 as the promoter operably linked to the FVII coding sequence.
         12. The plurality of engineered RPE cells of any one of embodiments 9 to 11, wherein the FVII protein is an FVII fusion protein.
         13. The plurality of engineered RPE cells of embodiment 12, wherein the FVII fusion protein comprises SEQ ID NO:11 or SEQ ID NO:12.
         14. The plurality of engineered RPE cells of any one of embodiments 9 to 13, wherein the exogenous nucleotide sequence comprises SEQ ID NO:13, SEQ ID NO:14, SEQ ID NO:15, SEQ ID NO:16, SEQ ID NO:17 or SEQ ID NO:18.
         15. The plurality of engineered RPE cells of any one of embodiments 9 to 14, wherein the exogenous nucleotide sequence comprises an extrachromosomal expression vector or is integrated into at least one location in the nuclear genome of the RPE cells.
         16. The plurality of engineered RPE cells of any one of embodiments 9 to 15, which are derived from ARPE-19 cells transfected with the isolated polynucleotide of any one of embodiments 1 to 8.
         17. The plurality of engineered RPE cells of any one of embodiments 9 to 16, which is provided as a polyclonal cell culture or as a culture of a monoclonal cell line.
         18. The plurality of engineered RPE cells of any one of embodiments 8 to 17, wherein the plurality exhibits one or more of the following features:
   a) secrete the FVII protein for at least 5 days, at least 10 days, at least one month, or at least two months, e.g., in an in vitro cell culture or when implanted into a subject (e.g., as evaluated by a reference method described herein); or
   b) secrete at least a 2-fold, 3-fold, 4-fold, 5-fold or 10-fold higher quantity of the FVII protein than a reference plurality of engineered RPE cells transfected with a wild-type human nucleotide sequence encoding precursor FVII.
      19. An implantable device which comprises at least one cell-containing compartment comprising the plurality of engineered RPE cells of any of embodiments 9 to 18 and at least one means for mitigating the foreign body response (FBR) when the device is implanted into the subject.
      20. The device of embodiment 19, wherein the at least one cell-containing compartment comprises a polymer composition which encapsulates the plurality of engineered RPE cells, and optionally comprises at least one cell-binding substance (CBS).
      21. The device of embodiment 19 or 20, wherein the cell-containing compartment comprises an alginate hydrogel and is surrounded by a barrier compartment, which comprises an alginate hydrogel and optionally comprises a compound of Formula (I), e.g., Compound 101 in Table 3, disposed on the outer surface of the barrier compartment.
      22. The device of embodiment 20 or 21, wherein the polymer composition comprises an alginate covalently modified with a peptide, wherein the peptide consists essentially of or consists of GRGDSP (SEQ ID NO:49), GGRGDSP (SEQ ID NO:51) or GGGRGDSP (SEQ ID NO:52).
      23. The device of any embodiment 21 or 22, wherein the barrier compartment comprises a mixture of an unmodified alginate and an alginate modified with Compound 101, wherein:
   (a) the unmodified alginate has a molecular weight of 150 kDa to 250 kDa and a G:M ratio of greater than or equal to 1.5; and
   (b) the alginate in the modified alginate has a molecular weight of <75 kDa and a G:M ratio of greater than or equal to 1.5.
      24. The device of embodiment 23, wherein the conjugation density of Compound 101 in the modified alginate is determined by quantitative free amine analysis, e.g., as described in Example 9 herein below, wherein the determined conjugation density is 1.0% w/w to 3.0% w/w, 1.3% w/w to 2.8% ww, 1.3% w/w to 2.6% w/w, 1.5% w/w to 2.4% w/w, 1.5% w/w to 2.2% w/w, or 1.7% w/w to 2.2% w/w.
      25. A hydrogel capsule comprising:
   (a) an inner cell-containing compartment which comprises the plurality of engineered cells of any one of embodiments 8 to 17 encapsulated in a first polymer composition comprising a first RGD-polymer, optionally wherein the concentration of the plurality of cells is 40 million cells per ml of the first polymer composition, or is any of 40 million to 100 million cells per ml, 60 million to 100 million cells per ml or 80 million to 100 million cells per ml of the first polymer composition; and
   (b) a barrier compartment surrounding the cell-containing compartment and comprising a second polymer composition which comprises a mixture of an unmodified alginate and an alginate covalently modified with at least one compound selected from the group consisting of Compound 100, Compound 101, Compound 110, Compound 112, Compound 113 and Compound 114 shown in Table 3,
      wherein the hydrogel capsule has a spherical shape and has a diameter of 0.5 millimeter to 5 millimeters, and optionally the average thickness of the barrier compartment is about 10 to about 300 microns, about 20 to about 150 microns, or about 40 to about 75 microns.
      26. The hydrogel capsule of embodiment 25, which comprises an effective amount of the first RGD-polymer for increased secretion of the Factor VII protein and wherein the first RGD-polymer consists essentially of an alginate covalently modified with an RGD peptide via a linker, the cell-containing compartment is substantially free of any afibrotic compound and the barrier compartment is substantially free of cells and the RGD peptide, and optionally wherein the effective amount of the RGD-polymer is an optimal amount.
      27. The hydrogel capsule of embodiment 25 or 26, wherein the RGD peptide consists essentially of an amino acid sequence of RGD (SEQ ID NO: 33) or RGDSP (SEQ ID NO: 48) and the linker is a single glycine residue or a single beta-alanine residue attached to the N-terminus of the RGD peptide.
      28. The hydrogel capsule of any one of embodiments 25 to 27, wherein:
   (a) the polymer in the first RGD-polymer is an alginate and has a molecular weight of 150 to 250 kDa and a G:M ratio of greater than or equal to 1.5, and optionally the cell-containing compartment is formed from an alginate solution with a viscosity of between about 90 cP and about 230 cP to about 300, 350 or 400 cP, or between about 80 cP to about 120 cP;
   (b) the alginate in the covalently-modified alginate in the barrier compartment has a molecular weight of <75 kDa and a G:M ratio of greater than or equal to 1.5;
   (c) the unmodified alginate in the barrier compartment has a molecular weight of 150 kDa to 250 kDa and a G:M ratio of greater than or equal to 1.5; and
   (d) optionally, the barrier compartment is formed from an alginate solution comprising a mixture of the covalently modified alginate and unmodified alginate and having a viscosity of 250-350 cP.
      29. The hydrogel capsule of embodiment 28, wherein:
   (a) the capsule has a diameter of between 1.0 millimeters and 2.0 millimeters;
   (b) the conjugation density of the RGD peptide on the alginate is the percent nitrogen determined by combustion analysis of the RGD-polymer (SEQ ID NO: 33) that has been lyophilized to a constant weight (e.g., as described in Example 1B herein), wherein the determined percent nitrogen is about 0.10% nitrogen (N) to 1.00% N, about 0.20% N to about 0.80% N, about 0.30% N to about 0.60% N, about 0.30% to about 0.50%, or 0.33% N to 0.46% N; and
   (c) the covalently modified alginate in the barrier compartment is modified only with Compound 101 and the density of Compound 101 in the covalently modified alginate is the percent nitrogen determined by combustion analysis of the covalently modified alginate that has been lyophilized to a constant weight, e.g., as described in Example 1A herein, wherein the determined percent nitrogen is between about nitrogen is at least 2.0% and less than 9.0%, or is 3.0% to 8.0%, 4.0% to 7.0%, 5.0% to 7.0%, or 6.0% to 7.0% or about 6.8%.
      30. The hydrogel capsule of embodiment 29, wherein:
   (a) the capsule has a diameter of about 1.5 millimeters;
   (b) the RGD peptide consists essentially of an amino acid sequence of RGDSP (SEQ ID NO: 48) and the linker is a single glycine residue;
   (c) the conjugation density of the RGD peptide on the alginate is selected from the group consisting of:
      (i) an amount effective to increase the viability of the engineered RPE cells, e.g., as determined by an assay described herein,
      (ii) an amount effective to increase the productivity of the engineered RPE cells, e.g., as determined by an assay described herein,
      (iii) the percent nitrogen determined by combustion analysis of the RGD-polymer that has been lyophilized to a constant weight (e.g., as described in Example 1B herein), wherein the determined percent nitrogen is about 0.10% nitrogen (N) to 1.00% N, about 0.20% N to about 0.80% N, about 0.30% N to about 0.60% N, about 0.30% to about 0.50%, or 0.33% N to 0.46% N;
      (iv) 0.1 to 1.0, 0.2 to 0.8, 0.3 to 0.7 or 0.3 to 0.6 micromoles of GRGDSP (SEQ ID NO: 49) per gram of RGD-polymer in solution, e.g., as described in Example 7 and optionally Example 8 herein; and
      (v) any combination of two or more of c(i), c(ii), c(iii) and c(iv); and
   (d) the covalently modified alginate in the barrier compartment is modified only with Compound 101 and the density of Compound 101 in the covalently modified alginate is:
      (i) the percent nitrogen determined by combustion analysis of the covalently modified alginate that has been lyophilized to a constant weight, e.g., as described in Example 1A herein, wherein the determined percent nitrogen is between about nitrogen is at least 2.0% and less than 9.0%, or is 3.0% to 8.0%, 4.0% to 7.0%, 5.0% to 7.0%, or 6.0% to 7.0% or about 6.8%; or
      (ii) the % amine on a weight/weight basis as determined by quantitative free amine analysis, e.g., as described in Example 9 herein, wherein the determined density is 1.0% w/w to 3.0% w/w, 1.3% w/w to 2.8% w/w, 1.3% w/w to 2.6% w/w, 1.5% w/w to 2.4% w/w, 1.5% w/w to 2.2% w/w, or 1.7% w/w to 2.2% w/w.
         31. The hydrogel capsule of embodiment 30, wherein the conjugation density of the RGD peptide on the alginate in the RGD-polymer is 0.3 to 0.6 micromoles of GRGDSP (SEQ ID NO: 49) per gram of RGD-polymer in solution.
         32. A preparation of devices, wherein each device in the preparation is a device of any one of embodiments 18 to 23.
         33. A composition comprising a plurality of hydrogel capsules, wherein each capsule in the composition is a hydrogel capsule of any one of embodiments 25 to 31.
         34. The composition of embodiment 33, wherein each capsule is a spherical hydrogel capsule with a diameter selected from the group consisting of: 0.5 millimeter to 2 millimeters; 0.7 millimeter to 1.8 millimeters, 1.0 millimeter to 1.8 millimeters; 1.2 millimeters to 1.7 millimeters; 1.3 millimeters to 1.7 millimeters; and 1.4 to 1.6 millimeters.
         35. The composition of embodiment 33 or 34, which is a pharmaceutically acceptable composition.
         36. A method of delivering a FVII therapy to a patient in need thereof, comprising administering an effective amount of the preparation of devices of embodiment 32 or the composition of any one of embodiments 33 to 35.
         37. The method of embodiment 36, wherein the patient has hemophilia.
         38. The method of embodiment 37, wherein the patient has been diagnosed as having a congenital FVII deficiency or an acquired FVII deficiency.
         39. A method of engineering a plurality of RPE cells to produce a FVII protein (e.g., a human FVII protein or variant thereof), having a property described herein, comprising stably transfecting the RPE cells with a polynucleotide as defined in any one of embodiments 1 to 8, and optionally isolating a monoclonal cell line expressing the FVII protein.
         40. The method of embodiment 39, wherein the plurality of RPE cells are derived from ARPE-19 cells.
         41. An engineered RPE cell capable of expressing and secreting an FVII protein, wherein the cell comprises an exogenous nucleotide sequence comprising a coding sequence for the FVII protein, wherein the coding sequence comprises a precursor FVII coding sequence selected from the group consisting of: (i) SEQ ID NO:3, (ii) a nucleotide sequence that is at least 95%, 96%, 97%, 98%, 99% or more identical to SEQ ID NO:3, (iii) SEQ ID NO:4, and (iv) a nucleotide sequence that is at least 95%, 96%, 97%, 98%, 99% or more identical to SEQ ID NO:4.
         42. The engineered RPE cell of embodiment 41, wherein the exogenous nucleotide sequence further comprises a promoter operably linked to the coding sequence, wherein the promoter optionally consists essentially of, or consists of, a nucleotide sequence that is identical to, or substantially identical to, SEQ ID NO:10 or SEQ ID NO:21.
         43. The engineered RPE cell of embodiment 42, wherein the coding sequence comprises SEQ ID NO:3 and the promoter consists of SEQ ID NO:10 or SEQ ID NO:21.
         44. The engineered RPE cell of embodiment 41, wherein the FVII protein is an FVII fusion protein, and optionally the FVII fusion protein comprises SEQ ID NO:11 or SEQ ID NO:12.
         45. The engineered RPE cell of embodiment 44, wherein the coding sequence comprises SEQ ID NO:13, SEQ ID NO:14, SEQ ID NO:15, SEQ ID NO:16, SEQ ID NO:17 or SEQ ID NO:18.
         46. The engineered RPE cell of embodiment 41, which comprises SEQ ID NO:23, SEQ ID NO:24 or SEQ ID NO:25.
         47. The engineered RPE cell of embodiment 41, which comprises SEQ ID NO:22.
         48. The engineered RPE cell of any one of embodiments 41 to 47, wherein the exogenous nucleotide sequence comprises an extrachromosomal expression vector or is integrated into at least one chromosomal location in the RPE cell.
         49. The engineered RPE cell of any one of embodiments 41 to 48, which is derived from an ARPE-19 cell.
         50. A composition comprising an engineered RPE cell of any one of embodiments 41 to 49.
         51. The composition of embodiment 50 which is a polyclonal cell culture or a culture of a monoclonal cell line.
         52. An isolated double-stranded DNA molecule which comprises a nucleotide sequence selected from the group consisting of: SEQ ID NO:3, SEQ ID NO:4, SEQ ID NO:13, SEQ ID NO:14, SEQ ID NO:15, SEQ ID NO:16, SEQ ID NO: 17, SEQ ID NO:18, SEQ ID NO:22, SEQ ID NO:23, SEQ ID NO:24 and SEQ ID NO:25.
         53. The isolated DNA molecule of embodiment 52, which consists essentially of SEQ ID NO:22.
         54. An implantable device comprising at least one cell-containing compartment which comprises an engineered RPE cell of any one of embodiments 41 to 49 and at least one means for mitigating the foreign body response (FBR) when the device is implanted into the subject.
         55. The implantable device of embodiment 54, wherein the at least one cell-containing compartment comprises a polymer composition which encapsulates the plurality of engineered RPE cells, and optionally comprises at least one cell-binding substance (CBS).
         56. The implantable device of embodiment 54 or 55, wherein the cell-containing compartment is surrounded by a barrier compartment comprising an alginate hydrogel and optionally a compound of Formula (I) disposed on the outer surface of the barrier compartment.
         57. The implantable device of embodiment 55 or 56, wherein the polymer composition comprises an alginate covalently modified with a peptide, wherein the peptide consists essentially of, or consists of, GRGDSP (SEQ ID NO:49) or GGRGDSP (SEQ ID NO:51), and wherein the barrier compartment comprises an alginate modified with

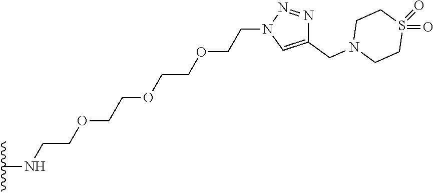 [SMILES: *NCCOCCOCCOCCn1cc(CN2CCS(=O)(=O)CC2)nn1]

or a pharmaceutically acceptable salt thereof.
58. A hydrogel capsule comprising:
(a) an inner compartment which comprises an engineered cell of any one of embodiments 41 to 49 encapsulated in a first polymer composition, wherein the first polymer composition comprises a hydrogel-forming polymer; and
(b) barrier compartment surrounding the inner compartment and comprising a second polymer composition, wherein the second polymer composition comprises an alginate covalently modified with at least one compound selected from the group consisting of Compound 100, Compound 101, Compound 110, Compound 112, Compound 113 and Compound 114 as shown in Table 3.
59. The hydrogel capsule of embodiment 58, wherein the selected compound is

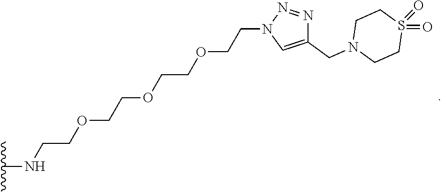 [SMILES: *NCCOCCOCCOCCn1cc(CN2CCS(=O)(=O)CC2)nn1]

60. The hydrogel capsule of embodiment 58 or 59, wherein the inner compartment comprises a plurality of the engineered cell of any one of embodiments 41 to 49, optionally wherein the concentration of the engineered cell in the inner compartment is at least 40 million cells per ml of the first polymer composition.
61. The hydrogel capsule of any one of embodiments 58 to 60, wherein the engineered cell is derived from an ARPE-19 cell and comprises SEQ ID NO:25.
62. A composition comprising a plurality of the hydrogel capsule of any one of embodiments 19 to 22.
63. A method of delivering a FVII therapy to a patient in need thereof, comprising administering the implantable device of any one of embodiments of 54 to 57, the hydrogel capsule of any one of embodiments 58 or 59, or the composition of embodiment 62.

### EXAMPLES

In order that the disclosure described herein may be more fully understood, the following examples are set forth. The examples described in this application are offered to illustrate the engineered RPE cells, implantable devices, and compositions and methods provided herein and are not to be construed in any way as limiting their scope.

### Example 1: Generation and Culturing of Exemplary Engineered ARPE-19 Cells

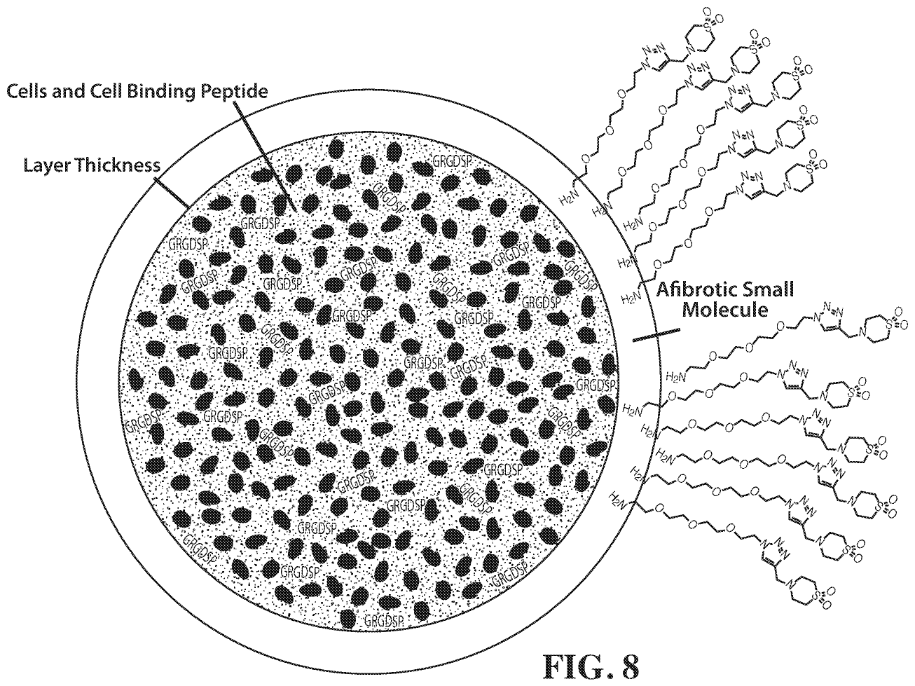

FVII-secreting cells were created using the expression vector shown in FIG. 8, in which an exogenous nucleotide sequence encoding a precursor FVII protein had been inserted using standard cloning methods. To accomplish this, ARPE-19 cells were co-transfected with a PiggyBac containing transposase plasmid along with an FVII-expression vector and the stably-transfected cells were cultured in complete growth medium containing puromycin. The FVII-expression vector was identical to the vector in FIG. 6, except for having one or two transcription units inserted between the 5′ ITR and 3′ ITR. Other than the promoter sequence and the inserted FVII coding sequence or FVII-albumin fusion sequence, the backbone sequence of each transcription unit was identical to the corresponding sequence shown in FIG. 6A and FIG. 6B, e.g., each transcription unit had the same Kozak sequence and the same rBG pA sequence. The promoter and FVII coding sequence combinations present in the FVII-expression vectors are listed below.

FVII-1 Vector: single transcription unit comprising a promoter (SEQ ID NO:10) operably linked to SEQ ID NO:3, which is a codon optimized coding sequence for wild-type, human precursor FVII.

FVII-2 Vector: single transcription unit comprising a promoter (SEQ ID NO:10) operably linked to SEQ ID NO:4, which is a codon optimized coding sequence for wild-type, human precursor FVII.

FVII-3 Vector: single transcription unit comprising a promoter (SEQ ID NO:10) operably linked to SEQ ID NO:2, which is a wild-type coding sequence for wild-type, human precursor FVII.

FVII-4 Vector: two transcription units, with the upstream unit comprising a promoter (SEQ ID NO:10) operably linked to SEQ ID NO:2 and the downstream unit comprising SEQ ID NO:21 fused to SEQ ID NO:3.

FVII-5 Vector: single transcription unit comprising a promoter (SEQ ID NO:10) operably linked to SEQ ID NO:3 fused to SEQ ID NO:8. SEQ ID NO:8 consists of a cleavable linker peptide fused to a codon optimized coding sequence for the mature human serum albumin protein.

FVII-6 Vector: single transcription unit comprising a promoter (SEQ ID NO:10) operably linked to SEQ ID NO:3 fused to SEQ ID NO:6. SEQ ID NO:6 consists of a glycine serine linker peptide fused to the same codon optimized coding sequence of the mature human serum albumin protein used in the FVII-5 vector.

FVII-7 Vector: two transcription units, with the upstream unit comprising a promoter (SEQ ID NO:10) operably linked to SEQ ID NO:3 and the downstream unit comprising a different promoter (SEQ ID NO:21) operably linked to SEQ ID NO:2.

FVII-8 Vector: two transcription units, with the upstream comprising a promoter (SEQ ID NO:10) operably linked to SEQ ID NO:3 fused to SEQ ID NO:8 and the downstream unit comprising SEQ ID NO:21 operably to SEQ ID NO:3 fused to SEQ ID NO:8.

The resulting stably-transfected ARPE-19 cells were cultured according to the following protocol. Cells were grown in complete growth medium (DMEM:F12 with 10% FBS) in 150 cm² cell culture flasks. To passage cells, the medium in the culture flask was aspirated, and the cell layer was briefly rinsed with phosphate buffered saline (pH 7.4, 137 mM NaCl, 2.7 mM KCl, 8 mM Na₂HPO₄, and 2 mM KH₂PO₄, Gibco). 5-10 mL of 0.05% (w/v) trypsin/0.53 mM EDTA solution (“TrypsinEDTA”) was added to the flask, and the cells were observed under an inverted microscope until the cell layer was dispersed, usually between 3-5 minutes. To avoid clumping, cells were handled with care and hitting or shaking the flask during the dispersion period was minimized. If the cells did not detach, the flasks were placed at 37° C. to facilitate dispersal. Once the cells dispersed, 10 mL complete growth medium was added, and the cells were aspirated by gentle pipetting. The cell suspension was transferred to a centrifuge tube and spun down at approximately 125×g for 5-10 minutes to remove TrypsinEDTA. The supernatant was discarded, and the cells were resuspended in fresh growth medium. Appropriate aliquots of cell suspension were added to new culture vessels, which were incubated at 37° C. The medium was renewed weekly.

### Example 2: Secretion of FVII from Exemplary Engineered ARPE-19 Cells

To quantify FVII protein secretion from cells, polyclonal populations of ARPE-19 cells engineered with various FVII-expression constructs as described in Example 1 were trypsinized as described above, and 500,000 cells were seeded in a single well of a 6-well tissue culture dish in 2 ml of growth medium. Engineered cells were allowed to settle and adhere to the dish for 3-4 hours before replacing the growth medium with fresh culture medium. One day (24 hours) after media replacement, the growth media containing secreted FVII was collected and placed on ice. Cells were washed as previously described with phosphate buffered saline and trypsinized with 0.05% TrypsinEDTA. Cells were collected and counted using trypan blue on a Countess II cell analyzer. FVII protein levels were determined using conditioned growth media from engineered cells mentioned above and run on a commercially available FVII ELISA kit (Abcam #190810). Total protein secretion was normalized to the total number of cells (picogram/cell/day), and the results are shown in FIG. 9.

### Example 3: Synthesis of Exemplary Compounds of Formula (I)

### General Protocols

The procedures below describe methods of preparing exemplary compounds for preparation of implantable devices described herein. The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Huisgen Cycloaddition to Afford 1,4-Substituted Triazoles

The copper-catalyzed Huisgen [3+2] cycloaddition was used to prepare triazole-based compounds and compositions, devices, and materials thereof. The scope and typical protocols have been the subject of many reviews (e.g., Meldal, M. and Tornoe, C. W. Chem. Rev. (2008) 108:2952-3015; Hein, J. E. and Fokin, V. V. Chem. Soc. Rev. (2010) 39(4):1302-1315; both of which are incorporated herein by reference).

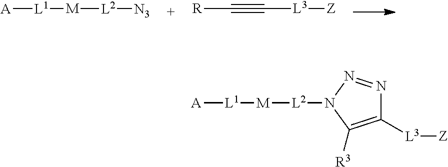

In the example shown above, the azide is the reactive moiety in the fragment containing the connective element A, while the alkyne is the reactive component of the pendant group Z. As depicted below, these functional handles can be exchanged to produce a structurally related triazole product. The preparation of these alternatives is similar, and do not require special considerations.

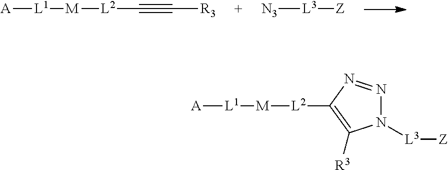

A typical Huisgen cycloaddition procedure starting with an iodide is outlined below. In some instances, iodides are transformed into azides during the course of the reaction for safety.

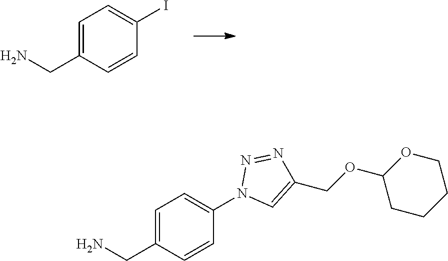 [SMILES: NCc1ccc(I)cc1 ; NCc1ccc(-n2cc(COC3CCCCO3)nn2)cc1]

A solution of sodium azide (1.1 eq), sodium ascorbate, (0.1 eq) trans-N,N′-dimethylcyclohexane-1,2-diamine (0.25 eq), copper (I) iodide in methanol (1.0 M, limiting reagent) was degassed with bubbling nitrogen and treated with the acetylene (1 eq) and the aryl iodide (1.2 eq). This mixture was stirred at room temperature for 5 minutes, then warmed to 55° C. for 16 h. The reaction was then cooled to room temperature, filtered through a funnel, and the filter cake washed with methanol. The combined filtrates were concentrated and purified via flash chromatography on silica gel (120 g silica, gradient of 0 to 40% (3% aqueous ammonium hydroxide, 22% methanol, remainder dichloromethane) in dichloromethane to afford the desired target material.

A typical Huisgen cycloaddition procedure starting with an azide is outlined below.

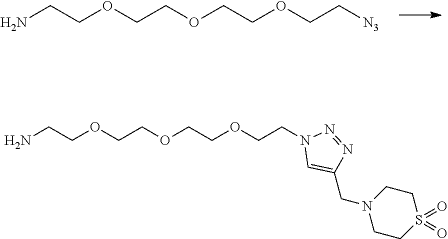 [SMILES: [N-]=[N+]=NCCOCCOCCOCCN ; NCCOCCOCCOCCn1cc(CN2CCS(=O)(=O)CC2)nn1]

A solution of tris[(1-benzyl-1H-1,2,3-triazol-4-yl)methyl]amine (0.2 eq), triethylamine (0.5 eq), copper (I) iodide (0.06 eq) in methanol (0.4 M, limiting reagent) was treated with the acetylene (1.0 eq) and cooled to 0° C. The reaction was allowed to warm to room temperature over 30 minutes, then heated to 55° C. for 16 h. The reaction was cooled to room temperature, concentrated, and purified with HPLC (C18 column, gradient of 0 to 100% (3% aqueous ammonium hydroxide, 22% methanol remainder dichloromethane) in dichloromethane to afford the desired target material.

### Huisgen Cycloaddition to Afford 1,5-Substituted Triazoles

The Huisgen [3+2] cycloaddition was also performed with ruthenium catalysts to obtain 1,5-disubstituted products preferentially (e.g., as described in Zhang et al, J. Am. Chem. Soc., 2005, 127, 15998-15999; Boren et al, J. Am. Chem. Soc., 2008, 130, 8923-8930, each of which is incorporated herein by reference in its entirety).

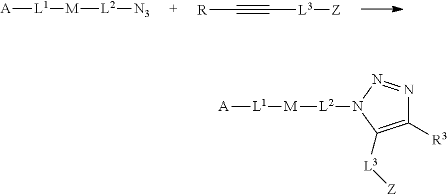

As described previously, the azide and alkyne groups may be exchanged to form similar triazoles as depicted below.

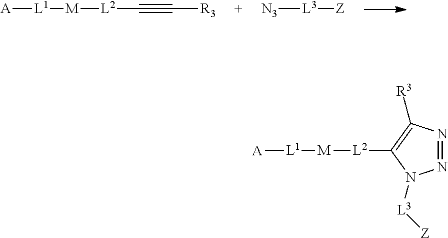

A typical procedure is described as follows: a solution of the alkyne (1 eq) and the azide (1 eq) in dioxane (0.8M) were added dropwise to a solution of pentamethylcyclo-pentadienylbis(triphenylphosphine) ruthenium(II) chloride (0.02 eq) in dioxane (0.16M). The vial was purged with nitrogen, sealed and the mixture heated to 60° C. for 12 h. The resulting mixture was concentrated and purified via flash chromatography on silica gel to afford the requisite compound.

### Experimental Procedure for (4-(4-((4-methylpiperazin-1-yl)methyl)-1H-1,2,3-triazol-1-yl)phenyl)methanamine (3)

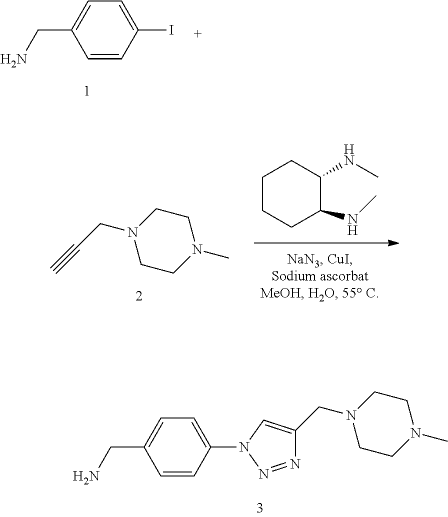

A mixture of (4-iodophenyl)methanamine (1, 843 mg, 3.62 mmol, 1.0 eq), (1S,2S)-N1,N2-dimethylcyclohexane-1,2-diamine (74 μL, 0.47 mmol, 0.13 eq), Sodium ascorbate (72 mg, 0.36 mmol, 0.1 eq), Copper Iodide (69 mg, 0.36 mmol, 0.1 eq), Sodium azide (470 mg, 7.24 mmol, 2.0 eq), and 1-methyl-4-(prop-2-yn-1-yl)piperazine (2, 0.5 g, 3.62 mmol, 1.0 eq) in Methanol (9 mL) and water (1 mL) were purged with nitrogen for 5 minutes and heated to 55° C. for overnight. The reaction mixture was cooled to room temperature, concentrated under reduced pressure, and the brownish slurry was extracted with dichloromethane. Celite was added to the combined dichloromethane phases and the solvent was removed under reduced pressure. The crude product was purified over silica gel (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 7.5% to afford (4-(4-((4-methylpiperazin-1-yl)methyl)-1H-1,2,3-triazol-1-yl)phenyl)methanamine (3, 0.45 g, 43%). LCMS m/z: [M+H]⁺ Calcd for C₁₅H₂₂N₆ 287.2; Found 287.1.

### Experimental Procedure for N-(4-(4-((4-methylpiperazin-1-yl)methyl)-1H-1,2,3-triazol-1-yl)benzyl)methacrylamide (4)

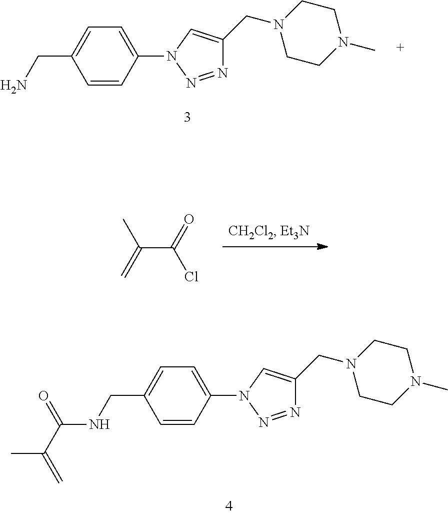

A solution of (4-(4-((4-methylpiperazin-1-yl)methyl)-1H-1,2,3-triazol-1-yl)phenyl)methanamine (3, 1.2 g, 4.19 mmol, 1.0 eq) and triethylamine (0.70 mL, 5.03 mmol, 1.2 eq) in CH₂Cl₂ (50 mL) was cooled to 0° C. with an ice-bath and methacryloyl chloride (0.43 mL, 4.40 mmol, 1.05 eq in 5 mL of CH₂Cl₂) was added. The reaction was stirred for a day while cooled with an ice-bath. Ten (10) grams of Celite were added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 7.5%. The solvent was removed under reduced pressure and the resulting solid was triturated with diethyl ether, filtered and washed multiple times with diethyl ether to afford N-(4-(4-((4-methylpiperazin-1-yl)methyl)-1H-1,2,3-triazol-1-yl)benzyl)methacrylamide (4, 0.41 g, 28% yield) as a white solid. LCMS m/z: [M+H]⁺ Calcd for C₁₉H₂₆N₆O 355.2; Found 355.2.

### Experimental Procedure for (4-(4-((2-(2-methoxyethoxy)ethoxy)methyl)-1H-1,2,3-triazol-1-yl)phenyl)methanamine (6)

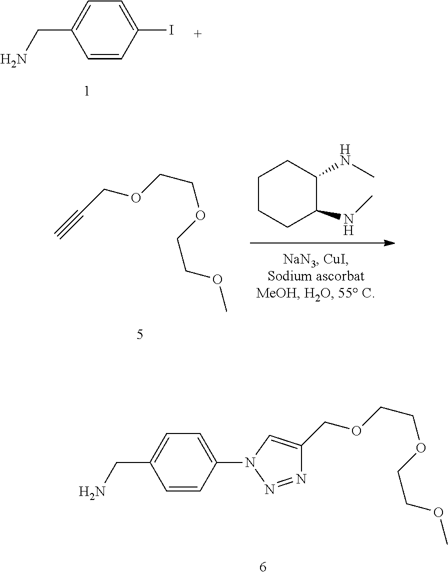

A mixture of (4-iodophenyl)methanamine (1, 2.95 g, 12.64 mmol, 1.0 eq), (1S,2S)-N1,N2-dimethylcyclohexane-1,2-diamine (259 μL, 1.64 mmol, 0.13 eq), Sodium ascorbate (250 mg, 1.26 mmol, 0.1 eq), Copper Iodide (241 mg, 1.26 mmol, 0.1 eq), Sodium azide (1.64 g, 25.29 mmol, 2.0 eq), and 1-methyl-4-(prop-2-yn-1-yl)piperazine (5, 2.0 g, 12.64 mmol, 1.0 eq) in Methanol (40 mL) and water (4 mL) were purged with Nitrogen for 5 minutes and heated to 55° C. overnight. The reaction mixture was cooled to room temperature and concentrated under reduced pressure. The residue was dissolved in dichloromethane, filtered, and concentrated with Celite (10 g). The crude product was purified by silica gel chromatography (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 6.25% to afford (4-(4-((2-(2-methoxyethoxy)ethoxy)methyl)-1H-1,2,3-triazol-1-yl)phenyl)methanamine (6, 1.37 g, 35%). LCMS m/z: [M+H]⁺ Calcd for C₁₅H₂₂N₄O₃ 307.2; Found 307.0.

### Experimental Procedure for N-(4-(4-((2-(2-methoxyethoxy)ethoxy)methyl)-1H-1,2,3-triazol-1-yl)benzyl)methacrylamide (7)

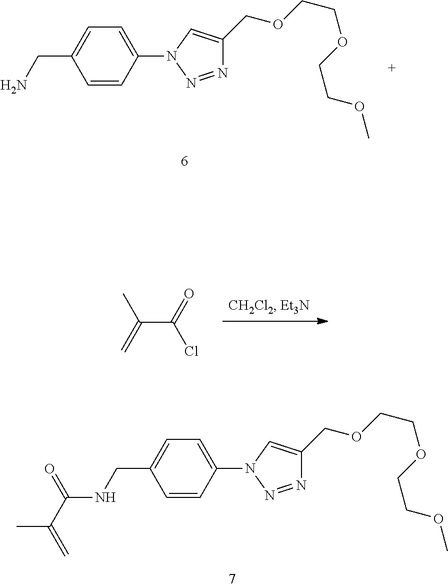

A solution of 4-(4-((2-(2-methoxyethoxy)ethoxy)methyl)-1H-1,2,3-triazol-1-yl)phenyl)methanamine (6, 1.69 g, 5.52 mmol, 1.0 eq) and triethylamine (0.92 mL, 6.62 mmol, 1.2 eq) in CH₂Cl₂ (50 mL) was cooled to 0° C. with an ice-bath and methacryloyl chloride (0.57 mL, 5.79 mmol, 1.05 eq) was added in a dropwise fashion. The reaction was stirred for 4 h at room temperature. Ten (10) grams of Celite were added and the solvent was removed under reduced pressure. The residue was purified by silica gel (80 g) chromatography using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(4-(4-((2-(2-methoxyethoxy)ethoxy)methyl)-1H-1,2,3-triazol-1-yl)benzyl)methacrylamide (7, 1.76 g, 85% yield) as a white solid. LCMS m/z: [M+H]⁺ Calcd for C₁₉H₂₆N₄O₄ 375.2; Found 375.0.

### Experimental Procedure for 3-(prop-2-yn-1-yloxy)oxetane (9)

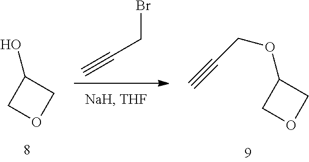

A suspension of sodium hydride (27.0 g, 675 mmol, 60% purity) in THE (200 mL) was cooled with an ice bath. Oexetan-3-ol (8, 25 g, 337 mmol) was added in a dropwise fashion and stirred for 30 minutes at 0° C. 3-Bromoprop1-yne (9, 41.2 mL, 371 mmol, 80% purity) was then added in a dropwise fashion. The mixture was stirred over night while allowed to warm to room temperature. The mixture was filtered over Celite, washed with THF, and concentrated with Celite under reduced pressure. The crude product was purified over silica gel (220 g) and eluted with Hexanes/EtOAc. The concentration of EtOAc in the mobile phase was increased from 0 to 25% to afford a yellow oil of (9, 18.25 g 48%).

### Experimental Procedure for 3-(4-((oxetan-3-yloxy)methyl)-1H-1,2,3-triazol-1-yl)propan-1-amine (11)

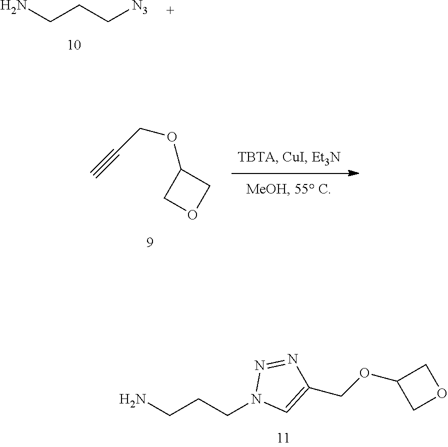

A mixture of 3-(prop-2-yn-1-yloxy)oxetane (9, 7.96 g, 71 mmol, 1.0 eq), 3-azidopropan-1-amine (10, 7.82 g, 78 mmol, 1.1 eq), Tris[(1-benzyl-1H-1,2,3-triazol-4-yl)methyl]-amine (8.29 g, 15.6 mmol, 0.22 eq), Copper Iodide (1.35 g, 7.1 mmol, 0.1 eq), and Triethylamine (2.47 mL, 17.8 mmol, 0.25 eq) in Methanol (80 mL) was warmed to 55° C. and stirred overnight under Nitrogen atmosphere. The reaction mixture was cooled to room temperature, Celite (20 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 15% to afford 3-(4-((oxetan-3-yloxy)methyl)-1H-1,2,3-triazol-1-yl)propan-1-amine (11, 11.85 g, 79%) as a yellow oil. LCMS m/z: [M+H]⁺ Calcd for C₉H₁₆N₄O₂ 213.1; Found 213.0.

### Experimental Procedure for N-(3-(4-((oxetan-3-yloxy)methyl)-1H-1,2,3-triazol-1-yl)propyl)methacrylamide (12)

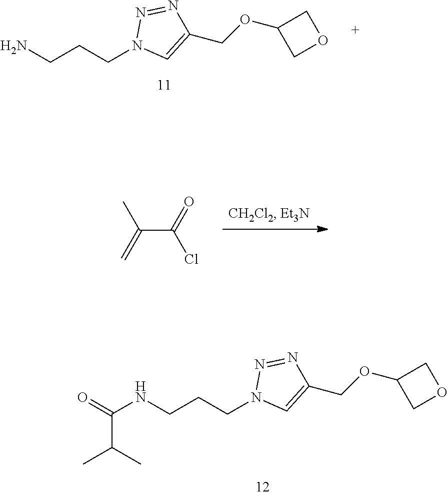

A solution of 3-(4-((oxetan-3-yloxy)methyl)-1H-1,2,3-triazol-1-yl)propan-1-amine (11, 3.94 g, 18.56 mmol, 1.0 eq) and triethylamine (3.1 mL, 22.28 mmol, 1.2 eq) in CH₂Cl₂ (100 mL) was cooled to 0° C. with an ice-bath and methacryloyl chloride (1.99 mL, 20.42 mmol, 1.1 eq) was added in a dropwise fashion. The reaction was stirred over night while allowed to warm to room temperature. 20 grams of Celite were added and the solvent was removed under reduced pressure. The residue was purified by silica gel chromatography (220 g) using dichloromethane/methanol as mobile phase. The concentration of methanol was gradually increased from 0% to 5% to afford N-(3-(4-((oxetan-3-yloxy)methyl)-1H-1,2,3-triazol-1-yl)propyl)methacrylamide (12, 3.22 g, 62% yield) as a solid. LCMS m/z: [M+H]⁺ Calcd for C₁₃H₂₀N₄O₃ 281.2; Found 281.0.

### Experimental Procedure for N-(4-(1H-1,2,3-triazol-1-yl)benzyl) methacrylamide (14)

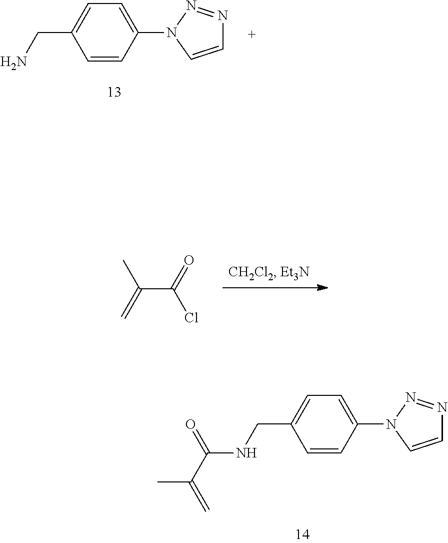

To a solution of (4-(1H-1,2,3-triazol-1-yl)phenyl)methanamine (13, obtained from WuXi, 1.2 g, 5.70 mmol, 1.0 eq) and triethylamine (15 mL, 107.55 mmol, 18.9 eq) in CH₂Cl₂ (100 mL) was slowly added methacryloyl chloride (893 mg, 8.54 mmol, 1.5 eq) in a dropwise fashion. The reaction was stirred overnight. 20 grams of Celite were added and the solvent was removed under reduced pressure. The residue was purified by silica gel chromatography using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(4-(1H-1,2,3-triazol-1-yl)benzyl) methacrylamide (14, 1.38 g, 40% yield).

### Experimental Procedure for (4-(4-(((tetrahydro-2H-pyran-2-yl)oxy)methyl)-1H-1,2,3-triazol-1-yl)phenyl)methanamine (15)

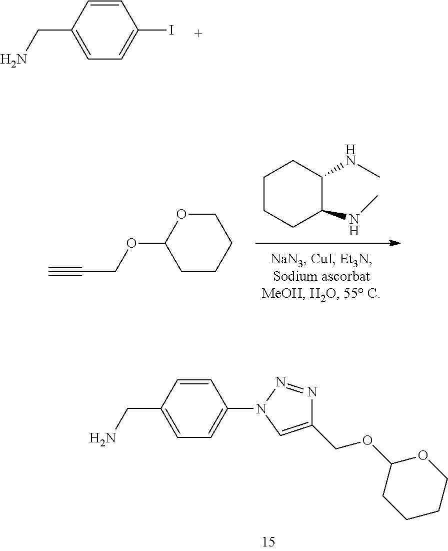

A mixture of (4-iodophenyl)methanamine hydrochloride (5.0 g, 18.55 mmol, 1.0 eq), (1S,2S)-N1,N2-dimethylcyclohexane-1,2-diamine (0.59 mL 3.71 mmol, 0.2 eq), Sodium ascorbate (368 mg, 1.86 mmol, 0.1 eq), Copper Iodide (530 mg, 2.78 mmol, 0.15 eq), Sodium azide (2.41 g, 37.1 mmol, 2.0 eq), Et₃N (3.11 mL, 22.26 mmol, 1.2 eq) and 2-(prop-2-yn-1-yloxy)tetrahydro-2H-pyran (2.6 g, 18.55 mmol, 1.0 eq) in Methanol (50 mL) and water (12 mL) were purged with Nitrogen for 5 minutes and heated to 55° C. for overnight. The reaction mixture was cooled to room temperature and filtered through 413 filter paper. Celite was added and the solvent was removed under reduced pressure and the residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 6.25% to afford (4-(4-(((tetrahydro-2H-pyran-2-yl)oxy)methyl)-1H-1,2,3-triazol-1-yl)phenyl)methanamine (15, 3.54 g, 66%) as a white solid. LCMS m/z: [M+H]⁺ Calcd for C₁₅H₂₀N₄O₂ 289.2; Found 289.2.

### Experimental Procedure for N-(4-(4-(((tetrahydro-2H-pyran-2-yl)oxy)methyl)-1H-1,2,3-triazol-1-yl)benzyl)methacrylamide (16)

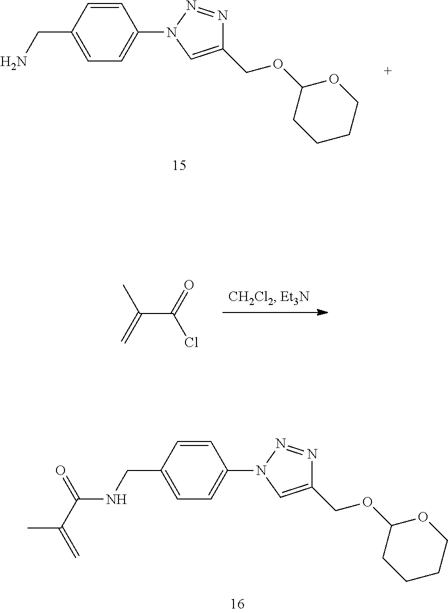

A solution of (4-(4-(((tetrahydro-2H-pyran-2-yl)oxy)methyl)-1H-1,2,3-triazol-1-yl)phenyl)methanamin (15, 3.46 g, 12.00 mmol, 1.0 eq) and triethylamine (2.01 mL, 14.40 mmol, 1.2 eq) in CH₂Cl₂ (40 mL) was cooled to 0° C. with an ice-bath and methacryloyl chloride (1.23 mL, 12.60 mmol, 1.05 eq, diluted in 5 mL of CH₂Cl₂) was added in a dropwise fashion. The cooling bath was removed, and the reaction was stirred for 4 h. 20 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 3.75% to afford N-(4-(4-(((tetrahydro-2H-pyran-2-yl)oxy)methyl)-1H-1,2,3-triazol-1-yl)benzyl)methacrylamide (16, 2.74 g, 64% yield) as a white solid. LCMS m/z: [M+H]⁺ Calcd for C₁₉H₂₄N₄O₃ 357.2; Found 357.3.

### Experimental Procedure for N-(4-(4-(hydroxymethyl)-1H-1,2,3-triazol-1-yl)benzyl)methacrylamide (17)

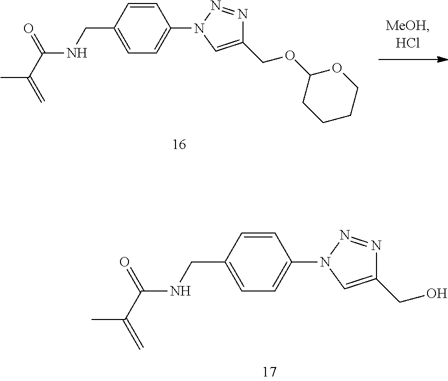 [SMILES: C=C(C)C(=O)NCc1ccc(-n2cc(COC3CCCCO3)nn2)cc1 ; C=C(C)C(=O)NCc1ccc(-n2cc(CO)nn2)cc1]

A solution of N-(4-(4-(hydroxymethyl)-1H-1,2,3-triazol-1-yl)benzyl)methacrylamide (16, 1.2 g, 3.37 mmol, 1.0 eq) was dissolved in Methanol (6 mL) and HCl (1N, aq., 9 mL) for overnight at room temperature. Celite was added and the solvent was removed under reduced pressure. The crude product was purified over silica gel chromatography (24 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 12.5% to afford N-(4-(4-(hydroxymethyl)-1H-1,2,3-triazol-1-yl)benzyl)methacrylamide (17, 0.85 g, 92% yield) as a white solid. LCMS m/z: [M+H]⁺ Calcd for C₁₄H₁₆N₄O₂ 273.1; Found 273.1.

### Experimental Procedure for (4-(((tetrahydro-2H-pyran-2-yl)oxy)methyl)benzyl)carbamate (19)

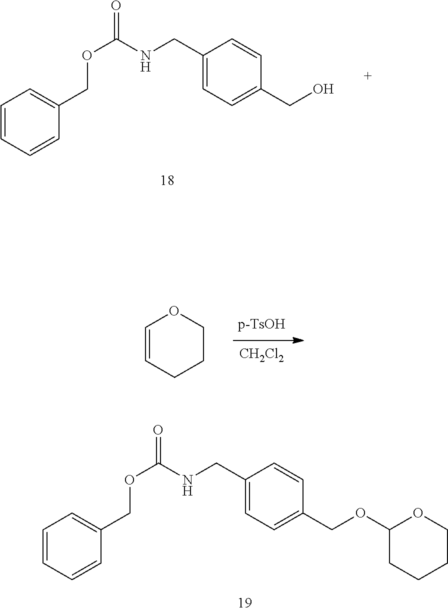

Benzyl (4-(hydroxymethyl)benzyl)carbamate (2.71 g, 10 mmol, 1 eq), 3,4-dihydro-2H-pyran (1.81 mL, 20 mmol, 2 eq), p-Toluenesulfonic acid monohydrate (285 mg, 1.5 mmol, 0.15 eq) in dichloromethane (100 mL) were stirred at room temperature overnight. Celite was added and the solvent was removed under reduced pressure. The crude product was purified over silica gel (24 g) using Hexanes/EtOAc as eluent starting at 100% Hexanes and increasing the concentration of EtOAc gradually to 100% to afford benzyl (4-(((tetrahydro-2H-pyran-2-yl)oxy)methyl)benzyl)-carbamate (19, 2.4 g, 68%) as a colorless oil. LCMS m/z: [M+Na]⁺ Calcd for C₂₁H₂₅NO₄ 378.17 Found 378.17.

### Experimental Procedure for (4-(((tetrahydro-2H-pyran-2-yl)oxy)methyl)-phenyl)methanamine (20)

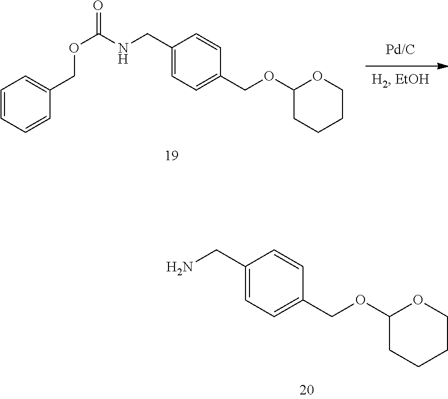

(4-(((tetrahydro-2H-pyran-2-yl)oxy)methyl)benzyl)carbamate (19, 1.5 g, 4.2 mmol, 1 eq), Palladium on carbon (160 mg, 10 wt. %) in EtOH was briefly evacuated and then Hydrogen was added via a balloon and the mixture was stirred for 1 hour at room temperature. Celite was added and the solvent was removed under reduced pressure. The crude product was purified over silica gel (12 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(((tetrahydro-2H-pyran-2-yl)oxy)methyl)phenyl)methanamine (20, 890 mg, 95%) as a colorless oil. LCMS m/z: [M+H]⁺ Calcd for C₁₃H₁₉NO₂ 222.15 Found 222.14.

### Experimental Procedure for N-(4-(((tetrahydro-2H-pyran-2-yl)oxy)methyl)benzyl)-methacrylamide (21)

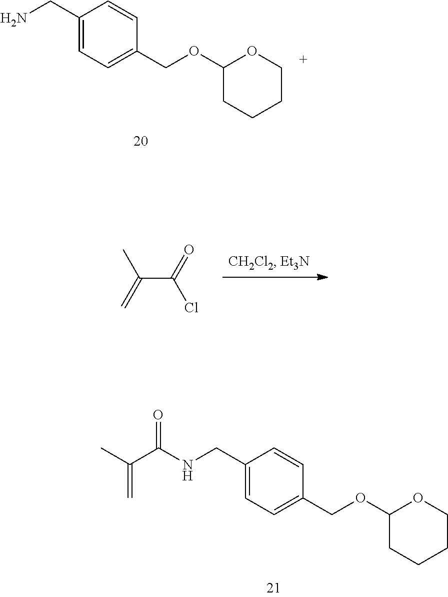

A solution of (4-(((tetrahydro-2H-pyran-2-yl)oxy)methyl)phenyl)methanamine (20, 0.5 g, 2.26 mmol, 1.0 eq) and triethylamine (0.47 mL, 3.39 mmol, 1.5 eq) in CH₂Cl₂ (10 mL) were briefly evacuated and flushed with Nitrogen. Methacryloyl chloride (0.33 mL, 3.39 mmol, 1.5 eq) was added in a dropwise fashion. The reaction mixture was stirred over night at room temperature. Ten (10) grams of Celite was added and the solvent was removed under reduced pressure. The residue was purified by silica gel chromatography (12 g) using Hexanes/EtOAc as eluent starting at 100% Hexanes and increasing the concentration of EtOAc gradually to 100% to afford N-(4-(((tetrahydro-2H-pyran-2-yl)oxy)methyl)benzyl)methacrylamide (21, 0.47 g, 72% yield) as a colorless solid. LCMS m/z: [M+Na]⁺ Calcd for C₁₇H₂₃NO₃ 312.16; Found 312.17.

### Experimental Procedure (4-(4-(2-((tetrahydro-2H-pyran-2-yl)oxy)ethyl)-1H-1,2,3-triazol-1-yl)phenyl)methanamine (22)

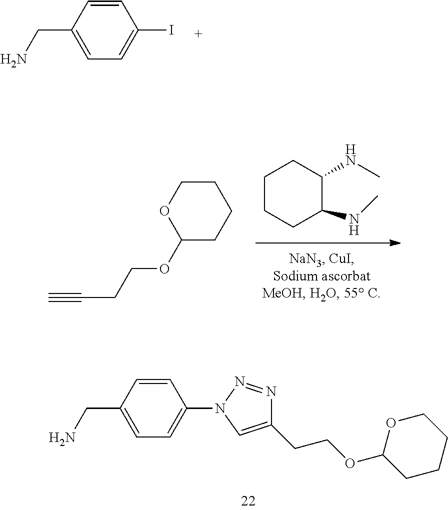

A mixture of (4-iodophenyl)methanamine (5.0 g, 21.45 mmol, 1.0 eq), (1S,2S)-N1,N2-dimethylcyclohexane-1,2-diamine (0.44 mL 2.79 mmol, 0.13 eq), Sodium ascorbate (425 mg, 2.15 mmol, 0.1 eq), Copper Iodide (409 mg, 2.15 mmol, 0.1 eq), Sodium azide (2.79 g, 42.91 mmol, 2.0 eq), and 2-(but-3-yn-1-yloxy)tetrahydro-2H-pyran (3.36 mL, 21.45 mmol, 1.0 eq) in Methanol (20 mL) and water (5 mL) were purged with Nitrogen for 5 minutes and heated to 55° C. for overnight. The reaction mixture was cooled to room temperature and filtered through 413 filter paper. Celite (10 g) was added and the solvent was removed under reduced pressure and the residue was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 5% to afford (4-(4-(2-((tetrahydro-2H-pyran-2-yl)oxy)ethyl)-1H-1,2,3-triazol-1-yl)phenyl)methanamine (22, 3.15 g, 49%) as a solid. LCMS m/z: [M+H]⁺ Calcd for C₁₆H₂₂N₄O₂ 303.18; Found 303.18.

### Experimental Procedure for N-(4-(4-(2-((tetrahydro-2H-pyran-2-yl)oxy)ethyl)-1H-1,2,3-triazol-1-yl)benzyl)methacrylamide (23)

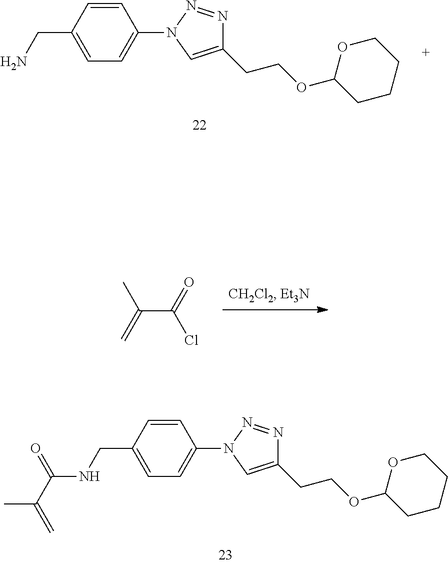

A solution of (4-(4-(2-((tetrahydro-2H-pyran-2-yl)oxy)ethyl)-1H-1,2,3-triazol-1-yl)phenyl)methanamine (22, 3.10 g, 10.25 mmol, 1.0 eq) and triethylamine (1.71 mL, 12.30 mmol, 1.2 eq) in CH₂Cl₂ (55 mL) was cooled to 0° C. with an ice-bath and methacryloyl chloride (1.05 mL, 12.30 mmol, 1.2 eq, diluted in 5 mL of CH₂Cl₂) was added in a dropwise fashion. The cooling bath was removed, and the reaction was stirred for 4 h. 8 grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 2.5% to afford N-(4-(4-(2-((tetrahydro-2H-pyran-2-yl)oxy)ethyl)-1H-1,2,3-triazol-1-yl)benzyl)methacrylamide (23, 2.06 g, 54% yield) as a white solid. LCMS m/z: [M+H]⁺ Calcd for C₂₀H₂₆N₄O₃ 371.2078; Found 371.2085.

### Experimental Procedure (4-(1-(2-((tetrahydro-2H-pyran-2-yl)oxy)ethyl)-1H-1,2,3-triazol-4-yl)phenyl)methanamine (24)

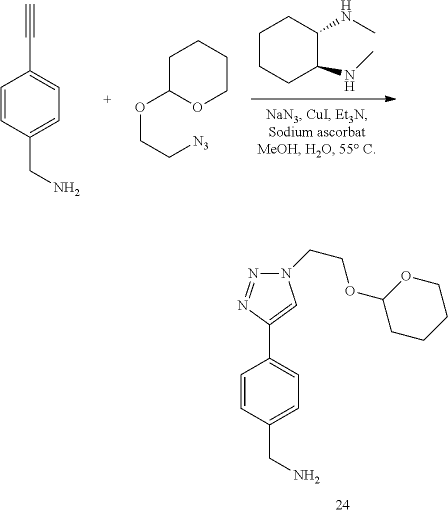

A mixture of (4-ethynylphenyl)methanamine (2.36 g, 18.00 mmol, 1.0 eq), (1S,2S)-N1,N2-dimethylcyclohexane-1,2-diamine (0.56 mL, 3.60 mmol, 0.2 eq), Sodium ascorbate (357 mg, 1.80 mmol, 0.1 eq), Copper Iodide (514 mg, 2.70 mmol, 0.15 eq), and 2-(2-azidoethoxy)tetrahydro-2H-pyran (3.08, 18.00 mmol, 1.0 eq) in Methanol (24 mL) and water (6 mL) were purged with Nitrogen for 5 minutes and heated to 55° C. for overnight. The reaction mixture was cooled to room temperature and filtered over Celite and rinsed with MeOH (3×50 mL). The solvent was removed under reduced pressure and the residue was redissolved in dichloromethane, Celite (20 g) was added and the solvent was removed under reduced pressure and the residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(1-(2-((tetrahydro-2H-pyran-2-yl)oxy)ethyl)-1H-1,2,3-triazol-4-yl)phenyl)methanamine (24, 3.51 g, 64%) as a yellowish oil. LCMS m/z: [M+H]⁺ Calcd for C₁₆H₂₂N₄O₂ 303.1816; Found 303.1814.

### Experimental Procedure for N-(4-(1-(2-((tetrahydro-2H-pyran-2-yl)oxy)ethyl)-1H-1,2,3-triazol-4-yl)benzyl)methacrylamide (25)

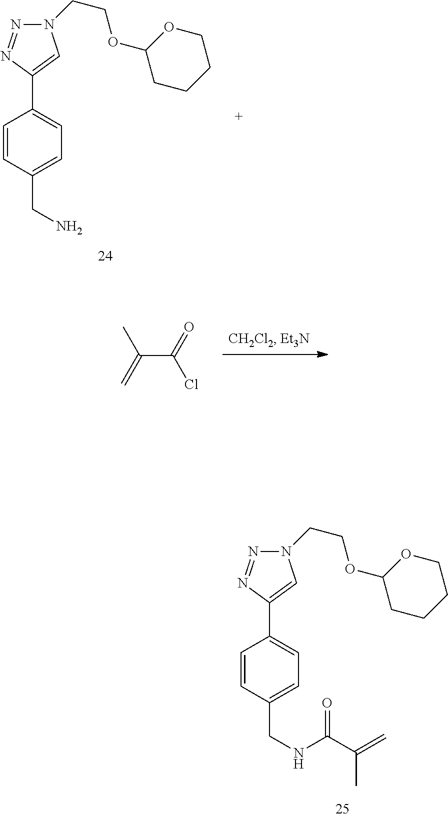

A solution of (4-(1-(2-((tetrahydro-2H-pyran-2-yl)oxy)ethyl)-1H-1,2,3-triazol-4-yl)phenyl)methanamine (24, 1.5 g, 4.96 mmol, 1.0 eq) and triethylamine (1.04 mL, 7.44 mmol, 1.5 eq) in CH₂Cl₂ (30 mL) were briefly evacuated and flushed with Nitrogen. Methacryloyl chloride (0.72 mL, 7.44 mmol, 1.5 eq) was added in a dropwise fashion. The reaction mixture was stirred for 2 h at room temperature. Ten (10) grams of Celite was added and the solvent was removed under reduced pressure. The residue was purified by silica gel chromatography (40 g) using Hexanes/EtOAc as eluent starting at 100% Hexanes and increasing the concentration of EtOAc gradually to 100% to afford N-(4-(1-(2-((tetrahydro-2H-pyran-2-yl)oxy)ethyl)-1H-1,2,3-triazol-4-yl)benzyl)methacrylamide (25, 0.9 g, 49% yield) as a colorless solid. LCMS m/z: [M+Na]⁺ Calcd for C₂₀H₂₆N₄O₃ 371.2078; Found 371.2076.

### Experimental Procedure for 1-(4-(4-(((tetrahydro-2H-pyran-2-yl)oxy)methyl)-1H-1,2,3-triazol-1-yl)phenyl)ethan-1-amine (26)

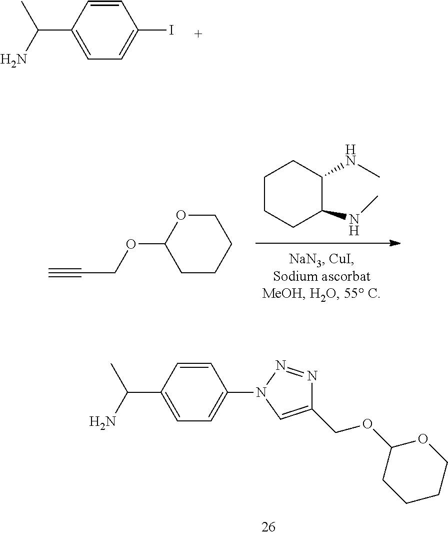

A mixture of 1-(4-iodophenyl)ethan-1-amine hydrochloride (1.0 g, 4.05 mmol, 1.0 eq), (1S,2S)-N1,N2-dimethylcyclohexane-1,2-diamine (0.08 mL 0.53 mmol, 0.13 eq), Sodium ascorbate (80 mg, 0.40 mmol, 0.1 eq), Copper Iodide (77 mg, 0.40 mmol, 0.1 eq), Sodium azide (526 g, 8.09 mmol, 2.0 eq), and 2-(prop-2-yn-1-yloxy)tetrahydro-2H-pyran (0.57 g, 4.05 mmol, 1.0 eq) in Methanol (9 mL) and water (1 mL) were purged with Nitrogen for 5 minutes and heated to 55° C. for overnight. The reaction mixture was cooled to room temperature and the solvent was removed under reduced pressure. The residue was redissolved in dichloromethane and filtered over a plug of Celite. Celite was added to the filtrate and the solvent was removed under reduced pressure. The residue was purified over silica gel (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 5% to afford 1-(4-(4-(((tetrahydro-2H-pyran-2-yl)oxy)methyl)-1H-1,2,3-triazol-1-yl)phenyl)ethan-1-amine (26, 0.62 g, 51%) as a yellowish solid. LCMS m/z: [M+H]⁺ Calcd for C₁₆H₂₂N₄O₂ 303.2; Found 303.2.

### Experimental Procedure for N-(1-(4-(4-(((tetrahydro-2H-pyran-2-yl)oxy)methyl)-1H-1,2,3-triazol-1-yl)phenyl)ethyl)methacrylamide (27)

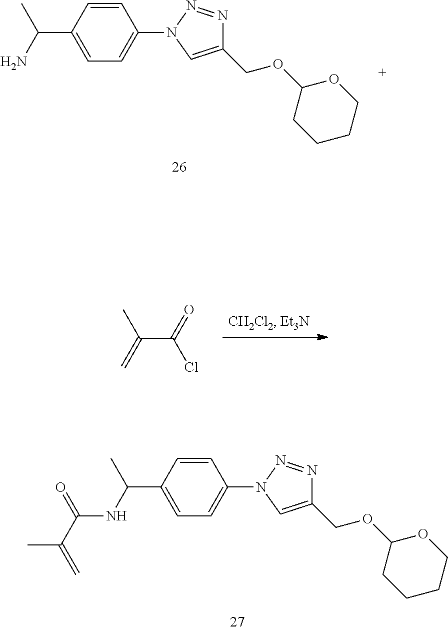

A solution of 1-(4-(4-(((tetrahydro-2H-pyran-2-yl)oxy)methyl)-1H-1,2,3-triazol-1-yl)phenyl)ethan-1-amine (26, 0.52 g, 1.7 mmol, 1.0 eq) and triethylamine (0.29 mL, 2.1 mmol, 1.2 eq) in CH₂Cl₂ (11 mL) was cooled to 0° C. with an ice-bath and methacryloyl chloride (0.18 mL, 1.8 mmol, 1.05 eq, diluted in 11 mL of CH₂Cl₂) was added in a dropwise fashion. The cooling bath was removed, and the reaction was stirred for 4 h. Five (5) grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 2.5% to afford N-(1-(4-(4-(((tetrahydro-2H-pyran-2-yl)oxy)methyl)-1H-1,2,3-triazol-1-yl)phenyl)ethyl)methacrylamide (27, 0.49 g, 76% yield) as a white solid. LCMS m/z: [M+H]⁺ Calcd for C₂₀H₂₆N₄O₃ 371.2078; Found 371.2087.

### Experimental Procedure for (4-(4-(((tetrahydro-2H-pyran-2-yl)oxy)methyl)-1H-1,2,3-triazol-1-yl)-2-(trifluoromethyl)phenyl)methanamine (28)

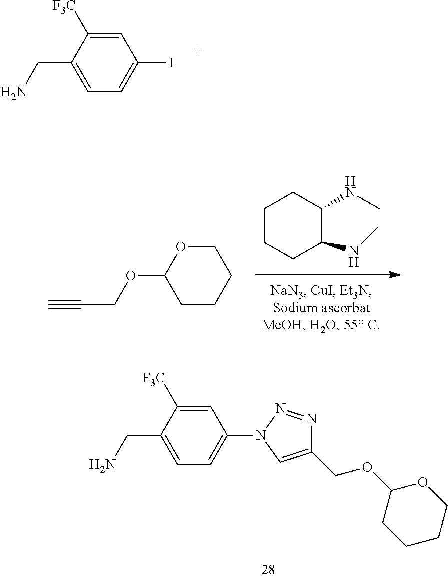

A mixture of (4-iodo-2-(trifluoromethyl)phenyl)methanamine (3.0 g, 9.97 mmol, 1.0 eq), (1S,2S)-N1,N2-dimethylcyclohexane-1,2-diamine (0.31 mL 1.99 mmol, 0.2 eq), Sodium ascorbate (197 mg, 1.00 mmol, 0.1 eq), Copper Iodide (285 mg, 1.49 mmol, 0.15 eq), Sodium azide (1.30 g, 19.93 mmol, 2.0 eq), Et₃N (1.67 mL, 11.96 mmol, 1.2 eq) and 2-(prop-2-yn-1-yloxy)tetrahydro-2H-pyran (1.40 g, 9.97 mmol, 1.0 eq) in Methanol (24 mL) and water (6 mL) were purged with Nitrogen for 5 minutes and heated to 55° C. for overnight. The reaction mixture was cooled to room temperature and filtered through a plug of Celite and rinsed with Methanol (3×50 mL). Celite was added to the filtrate and the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(4-(((tetrahydro-2H-pyran-2-yl)oxy)methyl)-1H-1,2,3-triazol-1-yl)-2-(trifluoromethyl)phenyl)methanamine (28, 2.53 g, 71%) as a green oil. LCMS m/z: [M+H]⁺ Calcd for C₁₆H₁₉N₄O₂F₃ 357.2; Found 357.1.

### Experimental Procedure for N-(4-(4-(((tetrahydro-2H-pyran-2-yl)oxy)methyl)-1H-1,2,3-triazol-1-yl)-2(trifluoromethyl)benzyl) methacrylamide (29)

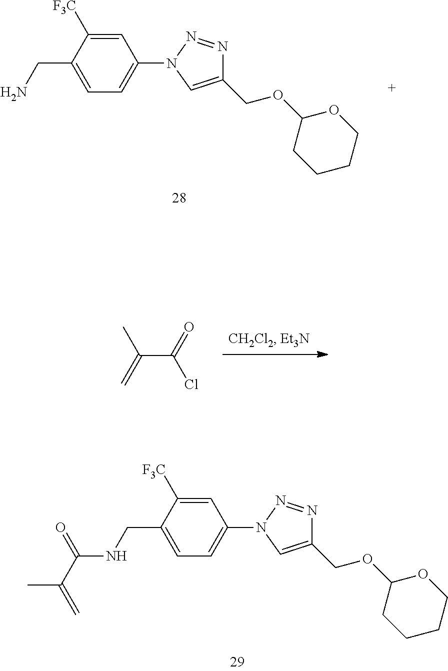

A solution of (4-(4-(((tetrahydro-2H-pyran-2-yl)oxy)methyl)-1H-1,2,3-triazol-1-yl)-2-(trifluoromethyl)phenyl) methanamine (28, 1.0 g, 2.81 mmol, 1.0 eq) and triethylamine (0.59 mL, 4.21 mmol, 1.5 eq) in CH₂Cl₂ (25 mL) were briefly evacuated and flushed with Nitrogen. Methacryloyl chloride (0.41 mL, 4.21 mmol, 1.5 eq) was added in a dropwise fashion. The reaction mixture was stirred for 6 h at room temperature. Ten (10) grams of Celite was added and the solvent was removed under reduced pressure. The residue was purified by silica gel chromatography (40 g) using Hexanes/EtOAc as eluent starting at 100% Hexanes and increasing the concentration of EtOAc gradually to 100% to afford N-(4-(4-(((tetrahydro-2H-pyran-2-yl)oxy)methyl)-1H-1,2,3-triazol-1-yl)-2(trifluoromethyl)benzyl) methacrylamide (29, 0.65 g, 55% yield) as a colorless solid. LCMS m/z: [M+H]⁺ Calcd for C₂₀H₂₃N₄O₃F₃ 425.2; Found 425.1.

### Experimental Procedure for 3-(4-(((tetrahydro-2H-pyran-2-yl)oxy)methyl)-1H-1,2,3-triazol-1-yl)propan-1-amine (30)

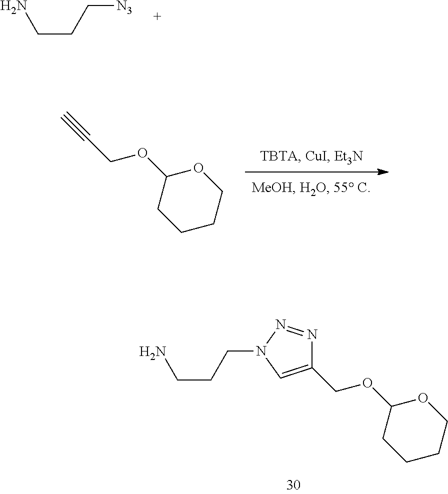

A mixture of 3-azidopropan-1-amine hydrochloride (1.5 g, 14.98 mmol, 1.0 eq), Tris[(1-benzyl-1H-1,2,3-triazol-4-yl)methyl]-amine (1.99 g, 3.75 mmol, 0.25 eq), Copper Iodide (0.29 g, 1.50 mmol, 0.1 eq), and Triethylamine (0.52 mL, 3.75 mmol, 0.25 eq) in Methanol (50 mL) and water (6 mL) were purged with Nitrogen for 5 minutes and cooled to 0 C. 2-(prop-2-yn-1-yloxy)tetrahydro-2H-pyran (2.10 g, 14.98 mmol, 1.0 eq) was added and the reaction mixture was warmed to 55° C. and stirred overnight under Nitrogen atmosphere. The reaction mixture was cooled to room temperature, filtered over a plug of Celite and rinsed with Methanol (3×50 mL). Celite (20 g) was added to the filtrate the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 20% to afford 3-(4-(((tetrahydro-2H-pyran-2-yl)oxy)methyl)-1H-1,2,3-triazol-1-yl)propan-1-amine (30, 2.36 g, 66%). LCMS m/z: [M+H]⁺ Calcd for C₁₁H₂₀N₄O₂ 241.2; Found 241.2.

### Experimental Procedure for N-(3-(4-(((tetrahydro-2H-pyran-2-yl)oxy)methyl)-1H-1,2,3-triazol-1-yl)propyl)methacrylamide (31)

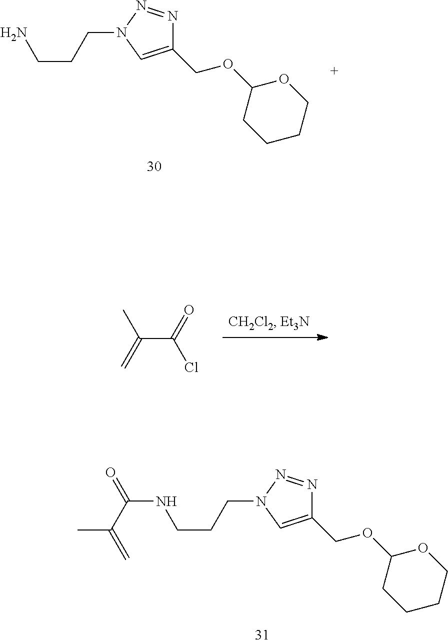

A solution of 3-(4-(((tetrahydro-2H-pyran-2-yl)oxy)methyl)-1H-1,2,3-triazol-1-yl)propan-1-amine (30, 1.0 g, 4.16 mmol, 1.0 eq) and triethylamine (0.58 mL, 4.16 mmol, 1.0 eq) in CH₂Cl₂ (20 mL) were briefly evacuated and flushed with Nitrogen. Methacryloyl chloride (0.40 mL, 4.16 mmol, 1.0 eq) was added in a dropwise fashion. The reaction mixture was stirred at room temperature overnight. Ten (10) grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 20% to afford N-(3-(4-(((tetrahydro-2H-pyran-2-yl)oxy)methyl)-1H-1,2,3-triazol-1-yl)propyl)methacrylamide (31, 0.96 g, 75% yield) as a colorless oil. LCMS m/z: [M+H]⁺ Calcd for C₁₅H₂₄N₄O₃ 309.2; Found 309.4.

### Experimental Procedure for (4-(4-((oxetan-3-yloxy)methyl)-1H-1,2,3-triazol-1-yl)phenyl)methanamine (32)

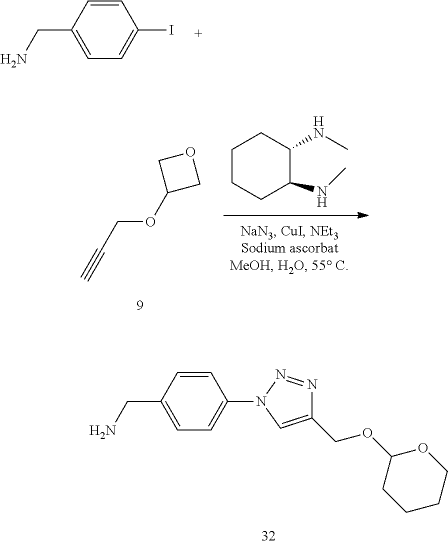

A mixture of (4-iodophenyl)methanamine hydrochloride (2.64 g, 9.80 mmol, 1.0 eq), (1S,2S)-N1,N2-dimethylcyclohexane-1,2-diamine (0.31 mL 1.96 mmol, 0.2 eq), Sodium ascorbate (198 mg, 0.98 mmol, 0.1 eq), Copper Iodide (279 mg, 1.47 mmol, 0.15 eq), Sodium azide (1.27 g, 19.59 mmol, 2.0 eq), Et₃N (1.64 mL, 11.75 mmol, 1.2 eq) and 3-(prop-2-yn-1-yloxy)oxetane (9, 1.10 g, 9.80 mmol, 1.0 eq) in Methanol (24 mL) and water (6 mL) were purged with Nitrogen for 5 minutes and heated to 55° C. for overnight. The reaction mixture was cooled to room temperature and filtered through a plug of Celite and rinsed with Methanol (3×50 mL). Celite was added to the filtrate and the solvent was removed under reduced pressure. The residue was purified over silica gel (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford (4-(4-((oxetan-3-yloxy)methyl)-1H-1,2,3-triazol-1-yl)phenyl)methanamine (32, 1.43 g, 56%) as an oil. LCMS m/z: [M+H]⁺ Calcd for C₁₃H₁₆N₄O₂ 261.1346; Found 261.1342.

### Experimental Procedure for N-(4-(4-((oxetan-3-yloxy)methyl)-1H-1,2,3-triazol-1-yl)benzyl)methacrylamide (33)

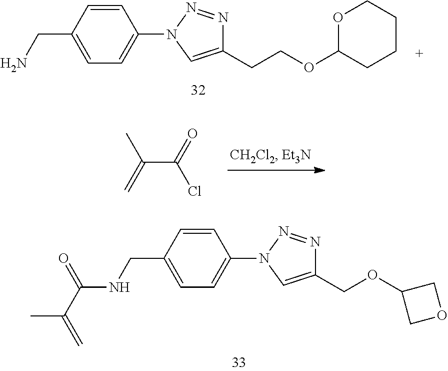

A solution of (4-(4-((oxetan-3-yloxy)methyl)-1H-1,2,3-triazol-1-yl)phenyl)methanamine (32, 0.58 g, 2.23 mmol, 1.0 eq) and triethylamine (0.47 mL, 3.34 mmol, 1.5 eq) in CH₂Cl₂ (20 mL) were briefly evacuated and flushed with Nitrogen. Methacryloyl chloride (0.32 mL, 3.34 mmol, 1.5 eq) was added in a dropwise fashion. The reaction mixture was stirred for 6 h at room temperature. Ten (10) grams of Celite was added and the solvent was removed under reduced pressure. The residue was purified by silica gel chromatography (24 g) using Hexanes/EtOAc as eluent starting at 100% Hexanes and increasing the concentration of EtOAc gradually to 100% to afford N-(4-(4-((oxetan-3-yloxy)methyl)-1H-1,2,3-triazol-1-yl)benzyl)methacrylamide (33, 0.48 g, 66% yield) as a colorless solid. LCMS m/z: [M+H]⁺ Calcd for C₁₇H₂₀N₄O₃ 329.1608; Found 329.1611.

### Experimental Procedure for ethyl 1-(2-methacrylamidoethyl)-1H-imidazole 4-carboxylate (35)

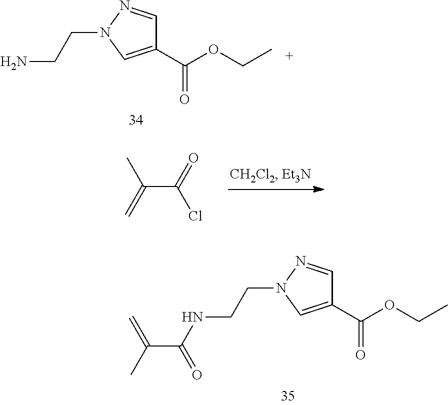

A solution of ethyl 1-(2-aminoethyl)-1H-imidazole-4-carboxylate (34, 2.0 g, 10.91 mmol, 1.0 eq) and triethylamine (3.80 mL, 27.29 mmol, 2.5 eq) in CH₂Cl₂ (20 mL) were briefly evacuated and flushed with Nitrogen. Methacryloyl chloride (1.60 mL, 16.37 mmol, 1.5 eq) was added in a dropwise fashion. The reaction mixture was stirred for 3 h at room temperature. Fifteen (15) grams of Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford ethyl 1-(2-methacrylamidoethyl)-1H-imidazole-4-carboxylate (35, 1.28 g, 47% yield) as a colorless solid. LCMS m/z: [M+H]⁺ Calcd for C₁₂H₁₇N₃O₃ 252.1; Found 252.1.

### Experimental Procedure for N-(4-(1,1-dioxidothiomorpholino)benzyl) methacrylamide (37)

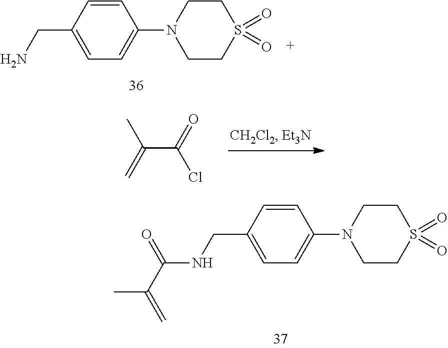

To a solution of 4-(4-(aminomethyl)phenyl)thiomorpholine 1,1-dioxide hydrochloride (36, 1.15 g, 4.15 mmol, 1.0 eq) and triethylamine (1.39 mL, 9.97 mmol, 2.4 eq) in CH₂Cl₂ (80 mL) was added a solution of methacryloyl chloride (0.43 mL, 4.36 mmol, 1.05 eq, in CH₂Cl₂, 5 mL) in a dropwise fashion. The reaction mixture was stirred for 22 h at room temperature. Eight (8) grams of Celite was added and the solvent was removed under reduced pressure. The residue was purified by silica gel chromatography (80 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 3.75% to afford N-(4-(1,1-dioxidothiomorpholino)benzyl) methacrylamide (37, 0.32 g, 25% yield) as a solid.

### Experimental Procedure for N-methyl-N-(2-(methylsulfonylethyl)prop-2-yn-1-amine (38)

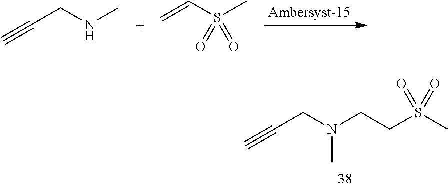

To a mixture of 1-methylsulfonylethylene (4.99 g, 47.03 mmol, 4.13 mL) and Amberlyst-15 ((30% w/w)), N-methylprop-2-yn-1-amine (2.6 g, 37.62 mmol) was added in a dropwise fashion. The mixture was stirred at room temperature for 12 hours. The catalyst was removed by filtration and the filtrate was concentrated under reduced pressure to afford: N-methyl-N-(2-(methylsulfonyl)ethyl)prop-2-yn-1-amine (38, 6.43 g, 98%) as an oil. LCMS m/z: [M+H]⁺ Calcd for C₇H₁₃NSO₂ 176.11; Found 176.1.

### Experimental Procedure for N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40)

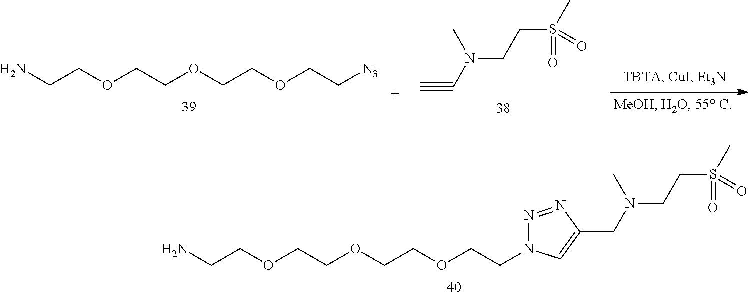

A mixture of N-methyl-N-(2-(methylsulfonyl)ethyl)prop-2-yn-1-amine (38, 5.02 g, 28.64 mmol, 1.25 eq), Tris[(1-benzyl-1H-1,2,3-triazol-4-yl)methyl]-amine (3.04 g, 5.73 mmol, 0.25 eq), Copper Iodide (436 mg, 2.29 mmol, 0.1 eq), and Triethylamine (0.8 mL, 5.7 mmol, 0.25 eq) in Methanol (50 mL) and water (6 mL) was evacuated and flushed with Nitrogen (3 times) and cooled with an ice bath. 2-(2-(2-(2-azidoethoxy)ethoxy)ethoxy)ethan-1-amine (39, 5.02 g, 22.91 mmol, 1.0 eq) was added in a dropwise fashion, the cooling bath was removed, and the mixture was stirred for 5 minutes. The reaction was warmed to 55° C. and stirred overnight under Nitrogen atmosphere. The reaction mixture was cooled to room temperature, Celite (20 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 25% to afford N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40, 4.98 g, 55%) as an oil. LCMS m/z: [M+H]⁺ Calcd for C₁₅H₃₁N₅O₅S 394.2; Found 394.2.

### Experimental Procedure N-(2-(2-(2-(2-(4-((methyl(2-(methylsulfonyl)ethyl)amino)methyl)-1H-1,2,3-triazol-1-yl)ethoxy)ethoxy)ethoxy)ethyl)methacrylamide (41)

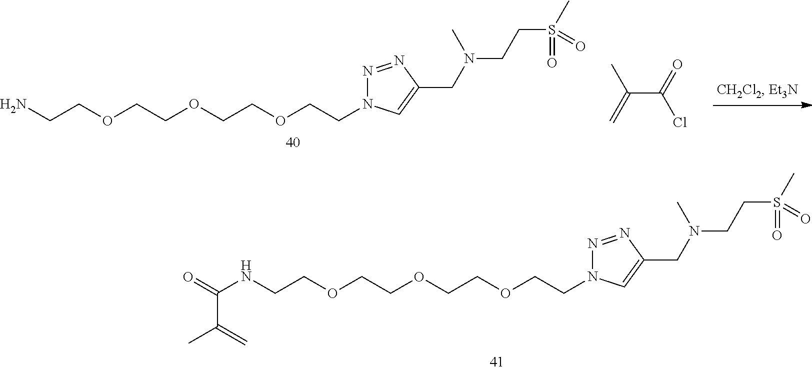

To a solution of N-((1-(2-(2-(2-(2-aminoethoxy)ethoxy)ethoxy)ethyl)-1H-1,2,3-triazol-4-yl)methyl)-N-methyl-2-(methylsulfonyl)ethan-1-amine (40, 1.0 g, 2.54 mmol, 1.0 eq) and triethylamine (0.43 mL, 3.05 mmol, 1.2 eq) in CH₂Cl₂ (15 mL) was added a solution of methacryloyl chloride (0.30 mL, 3.05 mmol, 1.5 eq) in a dropwise fashion. The reaction mixture was stirred for 5 h at room temperature. Celite was added and the solvent was removed under reduced pressure. The residue was purified by silica gel chromatography (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 12.5% to afford N-(2-(2-(2-(2-(4-((methyl(2-(methylsulfonyl)ethyl)amino)methyl)-1H-1,2,3-triazol-1-yl)ethoxy)ethoxy)ethoxy)ethyl)methacrylamide (41, 0.86 g, 73% yield) as an oil. LCMS m/z: [M+H]⁺ Calcd for C₁₉H₃₅N₅O₆S 462.2; Found 462.2.

### Experimental Procedure for 7-(prop-2-yn-1-yl)-2-oxa-7-azaspiro[3.5]nonane (42)

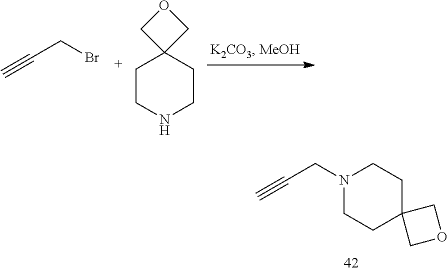

3-Bromoprop-1-yne (4.4 mL, 39.32 mmol 1.0 eq) was added to a mixture of 2-oxa-7-azaspiro[3.5]nonane (8.54 g, 39.32 mmol, 1.0 eq), potassium carbonate (17.9 g, 129.7 mmol, 3.3 eq) in Methanol (200 mL) and stirred over night at room temperature. The mixture was filtered, Celite was added and the solvent was removed under reduced pressure. The residue was purified by silica gel chromatography (220 g) using dichloromethane/methanol as mobile phase. The concentration of methanol was gradually increased from 0% to 5% to afford 7-(prop-2-yn-1-yl)-2-oxa-7-azaspiro[3.5]nonane (42, 4.44 g, 68%) as an oil.

### Experimental Procedure for 2-(2-(2-(2-(4-((2-oxa-7-azaspiro[3.5]nonan-7-yl)methyl)-1H-1,2,3-triazol-1-yl)ethoxy)ethoxy)ethoxy)ethan-1-amine (43)

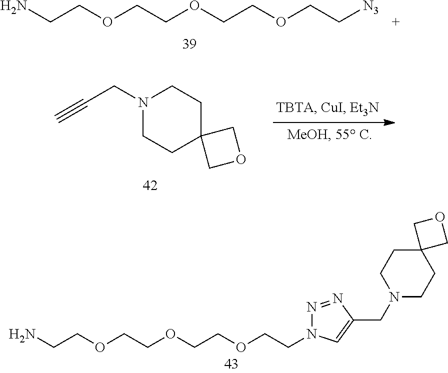 [SMILES: [N-]=[N+]=NCCOCCOCCOCCN ; C#CCN1CCC2(CC1)COC2 ; NCCOCCOCCOCCn1cc(CN2CCC3(CC2)COC3)nn1]

A mixture of 7-(prop-2-yn-1-yl)-2-oxa-7-azaspiro[3.5]nonane (42, 2.5 g, 15.13 mmol, 1.0 eq), Tris[(1-benzyl-1H-1,2,3-triazol-4-yl)methyl]-amine (1.77 g, 3.33 mmol, 0.22 eq), Copper Iodide (288 mg, 1.51 mmol, 0.1 eq), and Triethylamine (0.53 mL, 3.8 mmol, 0.25 eq) in Methanol (50 mL) was cooled with an ice bath. 2-(2-(2-(2-azidoethoxy)ethoxy)ethoxy)ethan-1-amine (39, 3.86 g, 17.70 mmol, 1.17 eq) was added in a dropwise fashion, the cooling bath was removed, and the mixture was stirred for 5 minutes. The reaction was warmed to 55° C. and stirred overnight under Nitrogen atmosphere. The reaction mixture was cooled to room temperature, Celite (10 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 2-(2-(2-(2-(4-((2-oxa-7-azaspiro[3.5]nonan-7-yl)methyl)-1H-1,2,3-triazol-1-yl)ethoxy)ethoxy)ethoxy)ethan-1-amine (43, 4.76 g, 82%) as an oil. LCMS m/z: [M+H]⁺ Calcd for C₁₈H₃₃N₅O₄ 384.3; Found 384.2.

### Experimental Procedure for N-(2-(2-(2-(2-(4-((2-oxa-7-azaspiro[3.5]nonan-7-yl)methyl)-1H-1,2,3-triazol-1-yl)ethoxy)ethoxy)ethoxy)ethyl)methacrylamide (44)

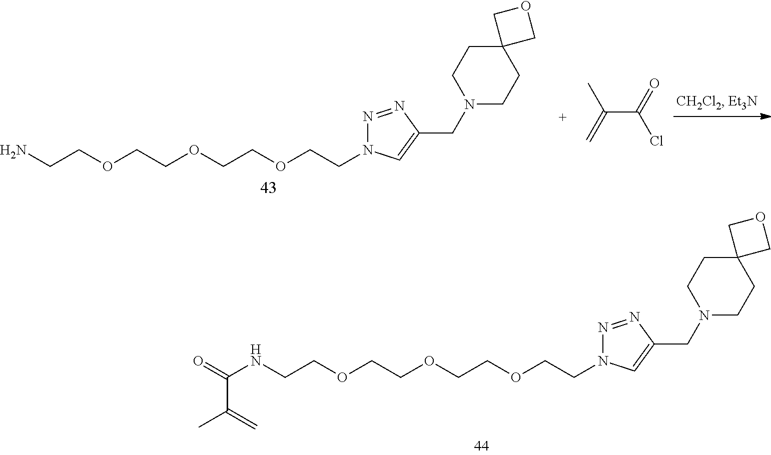

A solution of 2-(2-(2-(2-(4-((2-oxa-7-azaspiro[3.5]nonan-7-yl)methyl)-1H-1,2,3-triazol-1-yl)ethoxy)ethoxy)ethoxy)ethan-1-amine (43, 2.65 g, 6.91 mmol, 1.0 eq) and triethylamine (1.16 mL, 8.29 mmol, 1.2 eq) in CH₂Cl₂ (100 mL) was cooled with an ice-bath under Nitrogen atmosphere. Methacryloyl chloride (0.74 mL, 7.6 mmol, 1.1 eq) was added in a dropwise fashion. The cooling bath was removed, and the reaction mixture was stirred for 4 h at room temperature. Ten (10) grams of Celite was added and the solvent was removed under reduced pressure. The residue was purified by silica gel chromatography (120 g) using dichloromethane/methanol as mobile phase. The concentration of methanol was gradually increased from 0% to 10% to afford N-(2-(2-(2-(2-(4-((2-oxa-7-azaspiro[3.5]nonan-7-yl)methyl)-1H-1,2,3-triazol-1-yl)ethoxy)ethoxy)ethoxy)ethyl)methacrylamide (44, 1.50 g, 48% yield) as a colorless oil. LCMS m/z: [M+H]⁺ Calcd for C₂₂H₃₇N₅O₅ 452.29; Found 452.25.

### Experimental Procedure for 4-((1-(2-(2-aminoethoxy)ethyl)-1H-1,2,3-triazol-4-yl)methyl)thiomorpholine 1,1-dioxide (45)

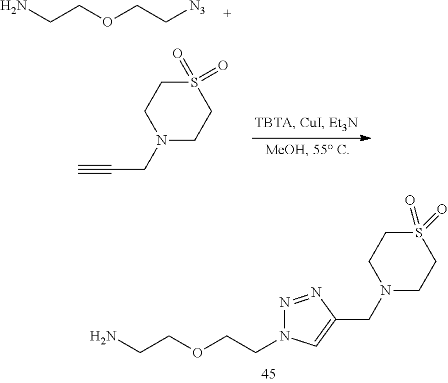

A mixture of 4-(prop-2-yn-1-yl)thiomorpholine 1,1-dioxide (1.14 g, 6.58 mmol, 1.0 eq), Tris[(1-benzyl-1H-1,2,3-triazol-4-yl)methyl]-amine (768 mg, 1.45 mmol, 0.22 eq), Copper Iodide (125 mg, 0.66 mmol, 0.1 eq), and Triethylamine (0.23 mL, 1.65 mmol, 0.25 eq) in Methanol (20 mL) was cooled with an ice bath. 2-(2-azidoethoxy)ethan-1-amine (1.00 g, 7.70 mmol, 1.17 eq) was added in a dropwise fashion, the cooling bath was removed, and the mixture was stirred for 5 minutes. The reaction was warmed to 55° C. and stirred overnight under Nitrogen atmosphere. The reaction mixture was cooled to room temperature, Celite (10 g) was added, and concentrated under reduced pressure. The crude product was purified over silica gel (40 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 9.5% to afford for 4-((1-(2-(2-aminoethoxy)ethyl)-1H-1,2,3-triazol-4-yl)methyl)thiomorpholine 1,1-dioxide (45, 1.86 g, 93%) as a white solid. LCMS m/z: [M+H]⁺ Calcd for C₁₁H₂₁N₅O₄S 304.1438; Found 304.1445.

### Experimental Procedure for N-(2-(2-(4-((1,1-dioxidothiomorpholino)methyl)-1H-1,2,3-triazol-1-yl)ethoxy)ethyl)methacrylamide (46)

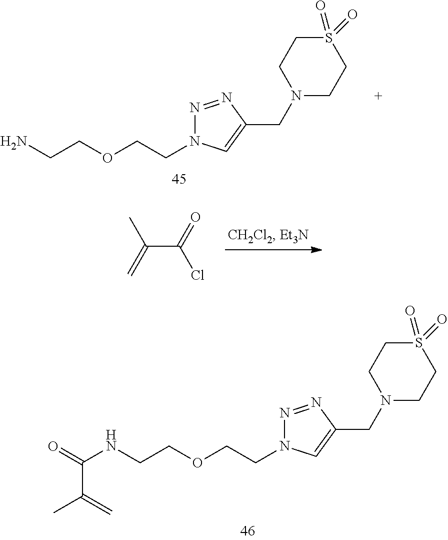

A solution of 4-((1-(2-(2-aminoethoxy)ethyl)-1H-1,2,3-triazol-4-yl)methyl)thiomorpholine 1,1-dioxide (45, 1.32 g, 4.35 mmol, 1.0 eq) and triethylamine (0.73 mL, 5.22 mmol, 1.2 eq) in CH₂Cl₂ (100 mL) was cooled with an ice-bath under Nitrogen atmosphere. Methacryloyl chloride (0.47 mL, 4.8 mmol, 1.1 eq) was added in a dropwise fashion. The cooling bath was removed, and the reaction mixture was stirred for 4 h at room temperature. Ten (10) grams of Celite was added and the solvent was removed under reduced pressure. The residue was purified by silica gel chromatography (120 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 1.25% to afford N-(2-(2-(4-((1,1-dioxidothiomorpholino)methyl)-1H-1,2,3-triazol-1-yl)ethoxy)ethyl)-methacrylamide (46, 0.90 g, 56% yield) as a colorless oil. LCMS m/z: [M+H]⁺ Calcd for C₁₅H₂₅N₅O₄S 372.17; Found 372.15.

### Experimental Procedure for 4-((1-(2-(2-(2-aminoethoxy)ethoxy)ethyl)-1H-1,2,3-triazol-4-yl)methyl)thiomorpholine 1,1-dioxide (47)

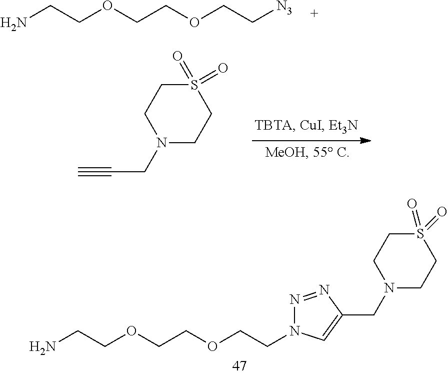

A mixture of 4-(prop-2-yn-1-yl)thiomorpholine 1,1-dioxide (4.6 g, 26.55 mmol, 1.0 eq), Tris[(1-benzyl-1H-1,2,3-triazol-4-yl)methyl]-amine (3.1 g, 5.84 mmol, 0.22 eq), Copper Iodide (506 mg, 2.66 mmol, 0.1 eq), and Triethylamine (0.93 mL, 6.64 mmol, 0.25 eq) in Methanol (80 mL) was cooled with an ice bath. 2-(2-(2-azidoethoxy)ethoxy)ethan-1-amine (5.00 g, 28.68 mmol, 1.08 eq) was added in a dropwise fashion, the cooling bath was removed, and the mixture was stirred for 5 minutes. The reaction was warmed to 55° C. and stirred overnight under Nitrogen atmosphere. The reaction mixture was cooled to room temperature, Celite was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 4-((1-(2-(2-(2-aminoethoxy)ethoxy)ethyl)-1H-1,2,3-triazol-4-yl)methyl)thiomorpholine 1,1-dioxide (47, 5.26 g, 57%) as a yellowish oil. LCMS m/z: [M+H]⁺ Calcd for C₁₃H₂₅N₅O₄S 348.1700; Found 348.1700.

### Experimental Procedure N-(2-(2-(2-(4-((1,1-dioxidothiomorpholino)methyl)-1H-1,2,3-triazol-1-yl)ethoxy)ethoxy)ethyl)methacrylamide (48)

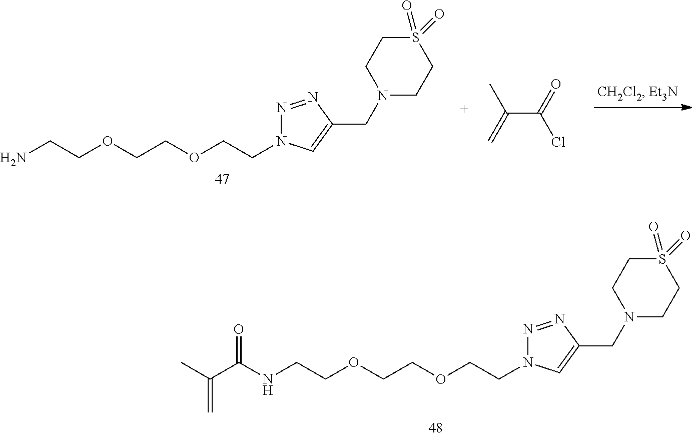

A solution of 4-((1-(2-(2-(2-aminoethoxy)ethoxy)ethyl)-1H-1,2,3-triazol-4-yl)methyl)thiomorpholine 1,1-dioxide (47, 1.49 g, 4.29 mmol, 1.0 eq) and triethylamine (0.72 mL, 5.15 mmol, 1.2 eq) in CH₂Cl₂ (50 mL) was cooled with an ice-bath under Nitrogen atmosphere. Methacryloyl chloride (0.46 mL, 4.7 mmol, 1.1 eq) was added in a dropwise fashion. The cooling bath was removed, and the reaction mixture was stirred for 4 h at room temperature. Ten (10) grams of Celite was added and the solvent was removed under reduced pressure. The residue was purified by silica gel chromatography (80 g) using dichloromethane/methanol as mobile phase. The concentration of methanol was gradually increased from 0% to 5% to afford N-(2-(2-(2-(4-((1,1-dioxidothiomorpholino)methyl)-1H-1,2,3-triazol-1-yl)ethoxy)ethoxy)ethyl)-methacrylamide (48, 0.67 g, 38% yield) as a colorless oil. LCMS m/z: [M+H]⁺ Calcd for C₁₇H₂₉N₅O₅S 416.20; Found 416.20.

### Experimental Procedure for 4-((1-(14-amino-3,6,9,12-tetraoxatetradecyl)-1H-1,2,3-triazol-4-yl)methyl)thiomorpholine 1,1-dioxide (49)

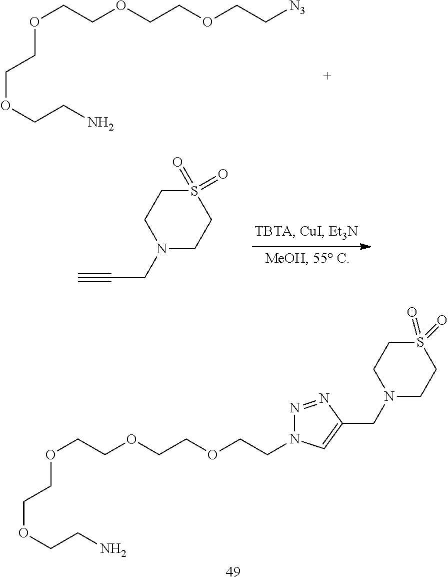 [SMILES: [N-]=[N+]=NCCOCCOCCOCCOCCN ; C#CCN1CCS(=O)(=O)CC1 ; NCCOCCOCCOCCOCCn1cc(CN2CCS(=O)(=O)CC2)nn1]

A mixture of 4-(prop-2-yn-1-yl)thiomorpholine 1,1-dioxide (5.0 g, 28.86 mmol, 1.0 eq), Tris[(1-benzyl-1H-1,2,3-triazol-4-yl)methyl]-amine (3.37 g, 6.35 mmol, 0.22 eq), Copper Iodide (550 mg, 2.89 mmol, 0.1 eq), and Triethylamine (1.01 mL, 7.22 mmol, 0.25 eq) in Methanol (90 mL) was cooled with an ice bath. 14-azido-3,6,9,12-tetraoxatetradecan-1-amine (8.86 g, 33.77 mmol, 1.17 eq) was added in a dropwise fashion, the cooling bath was removed, and the mixture was stirred for 5 minutes. The reaction was warmed to 55° C. and stirred overnight under Nitrogen atmosphere. The reaction mixture was cooled to room temperature, Celite (15 g) was added, and concentrated under reduced pressure. The crude product was purified over silica gel (220 g) using dichloromethane/(methanol containing 12% (v/v) aqueous ammonium hydroxide) as mobile phase. The concentration of (methanol containing 12% (v/v) aqueous ammonium hydroxide) was gradually increased from 0% to 10% to afford for 4-((1-(14-amino-3,6,9,12-tetraoxatetradecyl)-1H-1,2,3-triazol-4-yl)methyl)thiomorpholine 1,1-dioxide (49, 7.56 g, 60%) as an oil. LCMS m/z: [M+H]⁺ Calcd for C₁₇H₃₃N₅O₆S 436.2224; Found 436.2228.

### Experimental Procedure N-(14-(4-((1,1-dioxidothiomorpholino)methyl)-1H-1,2,3-triazol-1-yl)-3,6,9,12-tetraoxatetradecyl)methacrylamide (50)

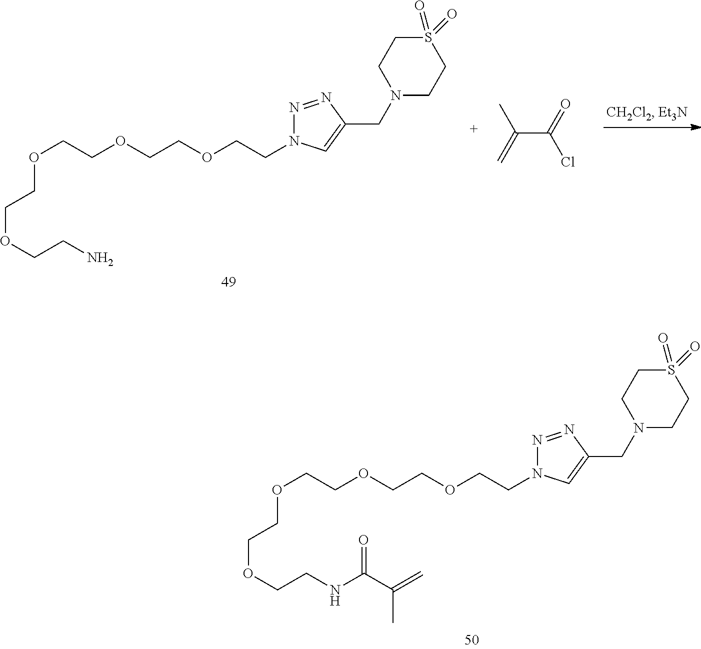

A solution of 4-((1-(14-amino-3,6,9,12-tetraoxatetradecyl)-1H-1,2,3-triazol-4-yl)methyl)thiomorpholine 1,1-dioxide (49, 1.95 g, 4.79 mmol, 1.0 eq) and triethylamine (0.80 mL, 5.74 mmol, 1.2 eq) in CH₂Cl₂ (50 mL) was cooled with an ice-bath under Nitrogen atmosphere. Methacryloyl chloride (0.51 mL, 5.26 mmol, 1.1 eq) was added in a dropwise fashion. The cooling bath was removed, and the reaction mixture was stirred for 4 h at room temperature. Ten (10) grams of Celite was added and the solvent was removed under reduced pressure. The residue was purified by silica gel chromatography (80 g) using dichloromethane/methanol as mobile phase. The concentration of methanol was gradually increased from 0% to 5% to afford N-(14-(4-((1,1-dioxidothiomorpholino)methyl)-1H-1,2,3-triazol-1-yl)-3,6,9,12-tetraoxatetradecyl)methacrylamide (50, 0.76 g, 32% yield) as a colorless oil. LCMS m/z: [M+H]⁺ Calcd for C₂₁H₃₇N₅O₇S 504.25; Found 504.20.

### Example 4A: Preparation of Exemplary Modified Polymers

1A. Chemically-modified Polymer. A polymeric material may be chemically modified with a compound of Formula (I) (or pharmaceutically acceptable salt thereof) prior to formation of a device described herein (e.g., a hydrogel capsule). Synthetic protocols of exemplary compounds for modification of polymeric materials are outlined above in Example 3. These compounds, or others, may be used to chemically modify any polymeric material.

For example, in the case of alginate, the alginate carboxylic acid is activated for coupling to one or more amine-functionalized compounds to achieve an alginate modified with an afibrotic compound, e.g., a compound of Formula (I). The alginate polymer is dissolved in water (30 mL/gram polymer) and treated with 2-chloro-4,6-dimethoxy-1,3,5-triazine (0.5 eq) and N-methylmorpholine (1 eq). To this mixture is added a solution of the compound of interest (e.g., Compound 101 shown in Table 3) in acetonitrile (0.3M).

The amounts of the compound and coupling reagent added depends on the desired concentration of the compound bound to the alginate, e.g., conjugation density. A medium conjugation density of Compound 101 typically ranges from 2% to 5% N, while a high conjugation density of Compound 101 typically ranges from 5.1% to 8% N. To prepare a CM-LMW-Alg-101-Medium polymer solution, the dissolved unmodified low molecular weight alginate (approximate MW<75 kDa, G:M ratio ≥1.5) is treated with 2-chloro-4,6-dimethoxy-1,3,5-triazine (5.1 mmol/g alginate) and N-methylmorpholine (10.2 mmol/g alginate) and Compound 101 (5.4 mmol/g alginate). To prepare a CM-LMW-Alg-101-High polymer solution, the dissolved unmodified low-molecular weight alginate (approximate MW<75 kDa, G:M ratio ≥1.5) is treated with 2-chloro-4,6-dimethoxy-1,3,5-triazine (5.1 mmol/g alginate) and N-methylmorpholine (10.2 mmol/g alginate) and Compound 101 (10.5 mmol/g alginate).

The reaction is warmed to 55° C. for 16 h, then cooled to room temperature and gently concentrated via rotary evaporation, then the residue is dissolved in water. The mixture is filtered through a bed of cyano-modified silica gel (Silicycle) and the filter cake is washed with water. The resulting solution is then extensively dialyzed (10,000 MWCO membrane) and the alginate solution is concentrated via lyophilization to provide the desired chemically-modified alginate as a solid or is concentrated using any technique suitable to produce a chemically modified alginate solution with a viscosity of 25 cP to 35 cP.

The conjugation density of a chemically modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

For use in generating exemplary hydrogel capsules encapsulating engineered ARPE-19 cells described above, chemically modified alginate polymers are prepared with Compound 101 (shown in Table 3) conjugated to a low molecular weight alginate (approximate MW<75 kDa, G:M ratio ≥0.5) at medium (2% to 5% N) or high (5.1% to 8% N) densities, as determined by combustion analysis for percent nitrogen, and are referred to herein as CM-LMW-Alg-101-Medium and CM-LMW-Alg-101-High.

1B. CBP-Alginates. A polymeric material may be covalently modified with a cell-binding peptide prior to formation of a device described herein (e.g., a hydrogel capsule described herein) using methods known in the art, see, e.g., Jeon O, et al., Tissue Eng Part A. 16:2915-2925 (2010) and Rowley, J. A. et al., Biomaterials 20:45-53 (1999).

For example, in the case of alginate, an alginate solution (1%, w/v) is prepared with 50 mM of 2-(N-morpholino)-ethanesulfonic acid hydrate buffer solution containing 0.5M NaCl at pH 6.5, and sequentially mixed with N-hydroxysuccinimide and 1-ethyl-3-[3-(dimethylamino)propyl]carbodiimide (EDC). The molar ratio of N-hydroxysuccinimide to EDC is 0.5:1.0. The peptide of interest is added to the alginate solution. The amounts of peptide and coupling reagent added depends on the desired concentration of the peptide bound to the alginate, e.g., peptide conjugation density. By increasing the amount of peptide and coupling reagent, higher conjugation density can be obtained. After reacting for 24 h, the reaction is purified by dialysis against ultrapure deionized water (diH2O) (MWCO 3500) for 3 days, treated with activated charcoal for 30 min, filtered (0.22 mm filter), and concentrated to the desired viscosity.

The conjugation density of a peptide-modified alginate is measured by combustion analysis for percent nitrogen. The sample is prepared by dialyzing a solution of the chemically modified alginate against water (10,000 MWCO membrane) for 24 hours, replacing the water twice followed by lyophilization to a constant weight.

In another embodiment, the conjugation density of a peptide-modified alginate is measured using a quantitative peptide-conjugation assay as described in Example 7 and optionally Example 8.

### Example 4B: Preparation of Exemplary Alginate Solutions for Making Hydrogel Capsules

70:30 mixture of chemically-modified and unmodified alginate. A low molecular weight alginate (PRONOVA™ VLVG alginate, NovaMatrix, Sandvika, Norway, cat. #4200506, approximate molecular weight <75 kDa; G:M ratio ≥1.5) is chemically modified with Compound 101 in Table 2 to produce chemically modified low molecular weight alginate (CM-LMW-Alg-101) solution with a viscosity of 25 cp to 35 cP and a conjugation density of 5.1% to 8% N, as determined by combustion analysis for percent nitrogen as described above. A solution of high molecular weight unmodified alginate (U-HMW-Alg) is prepared by dissolving unmodified alginate (PRONOVA™ SLG100, NovaMatrix, Sandvika, Norway, cat. #4202106, approximate molecular weight of 150 kDa-250 kDa) at 3% weight to volume in 0.9% saline. The CM-LMW-Alg solution is blended with the U-HMW-Alg solution at a volume ratio of 70% CM-LMW-Alg to 30% U-HMW-Alg (referred to herein as a 70:30 CM-Alg:UM-Alg solution).

Unmodified alginate solution. An unmodified medium molecular weight alginate (SLG20, NovaMatrix, Sandvika, Norway, cat. #4202006, approximate molecular weight of 75-150 kDa), is dissolved at 1.4% weight to volume in 0.9% saline to prepare a U-MMW-Alg solution.

Alginate Solution Comprising Cell Binding Sites. A solution of SLG20 alginate is modified with a peptide consisting of GRGDSP (SEQ ID NO: 49) and concentrated to a viscosity of about 100 cP as described in Example 4A above. In an embodiment, the amount of the GRGDSP peptide (SEQ ID NO: 49) and coupling reagent used are selected to achieve a target peptide conjugation density of about 0.2 to 0.3, as measured by combustion analysis as described in Example 4A above. In another embodiment, the amounts of a medium molecular weight alginate (approximate molecular weight of 75-150 kDa, G:M ratio of greater than or equal to 1.5), GRGDSP peptide (SEQ ID NO: 49) and coupling reagent used are selected to prepare a GRGDSP-MMW-Alg solution (“GRGDSP” disclosed as SEQ ID NO: 49) with a target peptide conjugation density of 0.3 to 0.6 micromoles of GRGDSP (SEQ ID NO: 49) per gram of the GRGDSP-MMW-Alg (“GRGDSP” disclosed as SEQ ID NO: 49) in saline with a viscosity of 80-120 cP.

### Example 5: Formation of Exemplary Two-Compartment Hydrogel Capsules

Suspensions of engineered ARPE-19 cells as single cells are encapsulated in two-compartment hydrogel capsules according to the protocols described below.

Immediately before encapsulation, engineered ARPE-19 cells are centrifuged at 1,400 r.p.m. for 1 min and washed with calcium-free Krebs-Henseleit (KH) Buffer (4.7 mM KCl, 25 mM HEPES, 1.2 mM KH₂PO₄, 1.2 mM MgSO₄×7H₂O, 135 mM NaCl, pH≈7.4, ≈290 mOsm). After washing, the cells ae centrifuged again and all of the supernatant is aspirated. The cell pellet is resuspended in the GRGDSP-modified alginate solution (“GRGDSP” disclosed as SEQ ID NO: 49) described in Example 4B at a desired cell density (e.g., about 50 to 150 million suspended single cells per ml alginate solution).

Prior to fabrication of hydrogel capsules, buffers and alginate solutions are sterilized by filtration through a 0.2-μm filter using aseptic processes.

To prepare particles configured as two-compartment hydrogel millicapsules of about 1.5 mm diameter, an electrostatic droplet generator is set up as follows: an ES series 0-100-kV, 20-watt high-voltage power generator (EQ series, Matsusada, N.C., USA) is connected to the top and bottom of a coaxial needle (inner lumen of 22 G, outer lumen of 18 G, Ramé-Hart Instrument Co., Succasunna, N.J., USA). The inner lumen is attached to a first 5-ml Luer-lock syringe (BD, NJ, USA), which is connected to a syringe pump (Pump 11 Pico Plus, Harvard Apparatus, Holliston, Mass., USA) that is oriented vertically. The outer lumen is connected via a luer coupling to a second 5-ml Luer-lock syringe which is connected to a second syringe pump (Pump 11 Pico Plus) that is oriented horizontally. A first alginate solution containing the engineered FVII-ARPE-19 cells (as single cells) suspended in a GRGDSP-modified alginate solution (“GRGDSP” disclosed as SEQ ID NO: 49) is placed in the first syringe and a cell-free alginate solution comprising a mixture of a chemically-modified alginate and unmodified alginate is placed in the second syringe. The two syringe pumps move the first and second alginate solutions from the syringes through both lumens of the coaxial needle and single droplets containing both alginate solutions are extruded from the needle into a glass dish containing a cross-linking solution. The settings of each Pico Plus syringe pump are 12.06 mm diameter and the flow rates of each pump are adjusted to achieve a flow rate ratio of 1:1 for the two alginate solutions. Thus, with the total flow rate set at 10 ml/h, the flow rate for each alginate solution was about 5 mL/h. Control (empty) capsules are prepared in the same manner except that the alginate solution used for the inner compartment is a cell-free solution.

After extrusion of the desired volumes of alginate solutions, the alginate droplets are crosslinked for five minutes in a cross-linking solution which contained 25 mM HEPES buffer, 20 mM BaCl₂, 0.2M mannitol and 0.01% of poloxamer 188. Capsules that fall to the bottom of the crosslinking vessel are collected by pipetting into a conical tube. After the capsules settle in the tube, the crosslinking buffer is removed, and capsules are washed. Capsules without cells may be washed four times with HEPES buffer (NaCl 15.428 g, KCl 0.70 g, MgCl₂.6H₂O 0.488 g, 0 ml of HEPES (1 M) buffer solution (Gibco, Life Technologies, California, USA) in 2 liters of deionized water) and stored at 4° C. until use. Capsules encapsulating cells may be washed four times in HEPES buffer, two times in 0.9% saline, and two times in culture media and stored in an incubator at 37° C.

The quality of capsules in a composition of two-compartment can be examined. For example, an aliquot containing at least 200 capsules is taken from the composition and transferred to a well plate and the entire aliquot examined by optical microscopy for quality by counting the number of spherical capsules out of the total.

### Example 6A: Exemplary Capsules for Delivering an FVII Protein

Compositions of two-compartment hydrogel millicapsules encapsulating ARPE-19 cells stably transfected with a transcription unit encoding a FVII protein (ARPE19:FVII) were prepared by extruding first and second alginate solutions through a coaxial needle substantially as described in Example 5. The second (outer) compartment was formed from the 70:30 CM-LMW-Alg-101-Medium:U-HMW-Alg solution (Example 4B) and the first (inner) compartment was formed from a U-MMW-Alg solution (Example 4B) or a GRGDSP-MMW-Alg solution (“GRGDSP” disclosed as SEQ ID NO: 49) with a viscosity of 80 to 120 cP and a GRGDSP (SEQ ID NO: 49) conjugation density ranging from: 0.12 to 1.98 micromoles (μmol) of GRGDSP (SEQ ID NO: 49) per gram of the GRGDSP-MMW-Alg (“GRGDSP” disclosed as SEQ ID NO: 49) (Example 4B). Each of the unmodified and GRGDSP-alginate solutions (“GRGDSP” disclosed as SEQ ID NO: 49) used to form the inner compartment contained a suspension of ARPE19:FVII cells at about 40 million cells per ml of alginate solution.

A 0.5 mL aliquot of each capsule composition was implanted into the IP space of nude mice (2 or 3 mice per composition). At 13 days post-implantation, animals were sacrificed, and blood samples were collected from each mouse. Plasma levels of FVII in these samples was measured by ELISA; the results are shown in FIG. 10.

Plasma levels of FVII were higher in mice implanted with capsules containing GRGDSP-alginate (SEQ ID NO: 49) with conjugation densities from 0.38 to 1.98 μmol/g than in mice implanted with capsules containing unmodified alginate or GRGDSP-alginate (SEQ ID NO: 49) with conjugation density of 0.12 or 0.21 μmol/g.

### Example 6B: In Vivo FVII Expression Following Implant of Encapsulated ARPE-19 Cells Engineered with Different Exemplary FVII-Expression Vectors

FVII-secreting cells were created by co-transfecting ARPE-19 cells with a PiggyBac containing transposase plasmid along with an FVII-expression vector and the stably-transfected cells (ARPE19:FVII cells) were cultured in complete growth medium containing puromycin. The FVII-expression vector was either the FVII-7 expression vector described in Example 1 above or the FVII-9 expression vector described in Table 4 below. The FVII-9 expression vector has two tandem transcription units with identical nucleotide sequences except that the promoter in the upstream transcription unit (relative to the 5′ ITR) consists of a CAG promoter (SEQ TD NO: 10) and the promoter in the downstream transcription unit consists of a CBh promoter (SEQ TD NO:21).

TABLE 5
FVII-9 Expression Vector (SEQ ID NO: 22)

| | Nucleotide Positions | Size | |
|---|---|---|---|
| Name | in SEQ ID NO: 22 | (bp) | Type |
| 5’ ITR | 1-313 | 313 | ITR |
| CAG | 337-2069 | 1733 | Promoter |
| R-U5’ | 2094-2375 | 282 | UTR |
| Kozak | 2376-2380 | 6 | Misc. |
| hFVII | 2382-3782 | 1401 | ORF |
| rBG pA | 3783-4304 | 522 | PolyA signal |
| CBh | 4334-5076 | 743 | Promoter |
| R-U5’ | 5101-5382 | 282 | UTR |
| Kozak | 5383-5388 | 6 | Misc. |
| hFVII | 5389-6789 | 1401 | ORF |
| rBG pA | 6808-7329 | 522 | PolyA signal |
| 3’ITR | Complement of | 235 | ITR |
| | 7514-7748 | | |
| AmpR | 8580-9440 | 861 | ORF |
| pUC ori | 9587-10258 | 672 | Rep origin |

Compositions of two-compartment hydrogel millicapsules encapsulating ARPE19:FVJJ-7 cells or ARPE19:FVJJ-9 cells were prepared by extruding first and second alginate solutions through a coaxial needle substantially as described in Example 5. The second (outer) compartment was formed from the 70:30 CM-LMW-Alg-101-Medium:U-HMW-Alg solution (Example 4B) and the first (inner) compartment was formed from a U-MMW-Alg solution (Example 4B) or a GRGDSP-MMW-Alg solution (“GRGDSP” disclosed as SEQ ID NO: 49) with a viscosity of 80 to 120 cP and a GRGDSP (SEQ ID NO: 49) conjugation density 0.44 micromoles (μmol) of GRGDSP (SEQ ID NO: 49) per gram of the GRGDSP-MMW-Alg (“GRGDSP” disclosed as SEQ ID NO: 49) (Example 4B). The GRGDSP-alginate solutions (“GRGDSP” disclosed as SEQ ID NO: 49) used to form the inner compartment contained a suspension of ARPE19:FVII cells at about 40 million cells per ml of alginate solution.

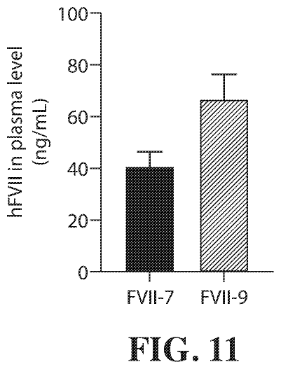

A 0.250 mL aliquot of each capsule composition was implanted into the IP space of nude mice (4 mice per composition). At 6 days post-implantation, animals were sacrificed, and blood samples were collected from each mouse. Plasma levels of FVII in these samples was measured by ELISA; the results are shown in FIG. 11.

Plasma levels of FVII were higher in mice implanted with capsules containing ARPE-19 cells transfected with the FVII-9 expression vector than in mice implanted with capsules containing ARPE-19 cells transfected with the FVII-7 expression vector.

### Example 7: Exemplary Quantitative Peptide Conjugation Assay

This assay determines the amount of peptide in a CBP-polymer by subjecting a sample of the CBP-polymer to acid hydrolysis, which cleaves off the CBP as individual amino acids. The individual amino acids in the hydrolyzed sample are separated and quantitated using amino acid references by pre-column on-line derivatization and reverse-phase liquid chromatography-Ultra-Violet-Fluoscense (LC-UV-FLR) (adapted from Agilent Biocolumns Amino Acid Analysis “How-To” Guide, Agilent Technologies, Inc., 5991-7694EN, published Mar. 1, 2018). Primary AAs (e.g., all but proline of the 20 standard L-alpha amino acids) are derivatized with Ortho-phthaladehyde (OPA) and secondary AAs (e.g., proline) are derivatized with 9-Fluorenylmethyl chloroformate (FMOC). The molar concentration of each amino acid is then averaged to calculate the concentration of the total peptide in the sample. This concentration can be corrected for the presence of any residual unconjugated CBP in the CBP-polymer by determining the amount of peptide in an unhydrolyzed sample of the CBP-polymer using any suitable analytical technique, e.g., as described in Example 8, and subtracting that amount from the total peptide amount. The assay is further described below as applied to determining peptide conjugation density in a GRGDSP-alginate (SEQ ID NO: 49); however, the skilled artisan can readily modify the assay to determine peptide concentration in a GRGDSP-alginate (SEQ TD NO: 49) or other peptide-modified polymers, provided the unmodified polymer does not contain any amino acids. Also, the skilled person can readily substitute any equipment, material or chemical specified below with a different equipment, material or chemical that can perform or provide substantially the same function or role in the assay.

### Definitions

| Abbreviation | Definition |
|---|---|
| LC-UV-FLR | Liquid Chromatography-Ultra-Violet-Fluorescence |
| LCMS | Liquid Chromatography-Mass Spectroscopy |
| SLG20 | Pronova Ultrapure SLG20 sterile sodium alginate |
| RT | Retention Time |
| ACN | Acetonitrile |
| MeOH | Methanol |
| SST | System Suitability |
| RSD | Relative Standard Deviation |
| TBD | To Be Determined |
| NMT | No More Than |
| NLT | No Less Than |
| RSQ | Coefficient of Determination |
| AA | Amino acid |
| OPA | Ortho-phthaladehyde |
| FMOC | 9-Fluorenylmethyl chloroformate |
| G | Glycine |
| R | Arginine |
| D | Aspartic acid |
| S | Serine |
| P | Proline |
| iSTD | Internal standard |
| PPE | Personal Protective Equipment |
| SDS | Safety Data Sheet |
| MW | Molecular Weight |
| PTFE | Polytetrafluoroethylene |
| RPM | Revolutions per Minute |
| min | Minutes |
| s | Seconds |
| mL | Millilitre |
| μL | Microlitre |
| nm | Nanometre |

### Equipment

Agilent 1260 LC system
Agilent diode array detector (G1315D): 13 μL/10 mm flow cell
Agilent Fluorescence detector (G1321B)
AdvanceBio AA LC column, 2.7 μm, 4.6×100 mm, Agilent 655950-80
AdvanceBio AAA guard column, 2.7 μm, 4.6×5 mm, Agilent 820750-931

| | | | |
|---|---|---|---|
| AA standard (17 AA): 25 pmol/μL | n/a | Agilent 5061-3333 | 2-8° C. |
| AA standard (17 AA): 100 pmol/uL | n/a | Agilent 5061-3332 | 2-8° C. |
| AA standard (17 AA): 250 pmol/uL | n/a | Agilent 5061-3331 | 2-8° C. |

### Procedure

Prepare 10 mM Na₂HPO₄/Na₂B₄O₇/pH 8.2 (aqueous mobile phase) and 45/45/10 ACN/MeOH/water (organic mobile phase) solution for use in the LC/MS procedure.

### Acid Hydrolysis of a Sample of an Exemplary Lyophilized Peptide-Alginate Conjugate

Weigh 12-16 mg of the lyophilized peptide-alginate conjugate into a microwave reaction vial, ensuring that the sample is not agitated.
Hydrolyze according to steps 3.3.2-3.3.19 below

### Acid Hydrolysis of an Exemplary Sample of a Peptide-Alginate Conjugate in Saline Solution

Weigh 1000±50 mg of the peptide-alginate conjugate solution in saline into a microwave reaction vial
Add 10.0 mL of 6N HCl to the sample using a 10-mL transfer pipet or volumetric pipet and a stir bar, and seal the PTFE-lined cap with a crimper
Place each sample vial in the matching heat block on a hot/stir plate
Heat at 120° C., stirring at 400 rpm, for 6 hours
Remove from heat and let cool to ambient temperature
Remove cap and transfer the entire solution from the reaction vial to a 20-mL volumetric flask with a disposable transfer pipet
Pipet 2 mL of LCMS grade water into the empty reaction vial, rinse the inner wall thoroughly with the same disposable transfer pipet, and transfer the rinseate completely to the same 20-mL volumetric flask
Repeat the above step twice
Bring to mark of the volumetric flask with LCMS grade water
Cap and invert the flask multiple times to mix well
Transfer completely to a 50-mL centrifuge tube
Centrifuge at 5000 rpm for 10 minutes
Pipet accurately 1 mL of the supernatant to an LC vial and store at 2-8° C. until drying (e.g., the next day) (store the remaining supernatant at 2-8° C. for any repeat testing if needed)
Dry the 1 mL supernatant completely under nitrogen at 60° C., make sure the needle does not touch the sample but is low enough for fast drying of the sample
Into the vial with the dried sample, pipet 0.25 mL of 0.1 μmol/mL internal standard mixture
Vortex thoroughly
Transfer with a pipet to an LC vial with a LC vial insert
Store at 2-8° C. until HPLC analysis (step 3.6)

### Standard Preparation

AA Standard Stock Solutions: 10 μmol/mL
For each AA, calculate the weight needed to prepare a 10 μmol/mL stock solution based on the MW
See an example below:

| | | | | | | Actual |
|---|---|---|---|---|---|---|
| | | | Actual | Vol- | | Concen- |
| Letter | | MW | weight | ume | Purity | tration |
| name | Full name | (g/mol) | (mg) | (mL) | (%) | (μmol/mL) |
| D | Aspartic acid | 133.10 | 66.38 | 50 | 100 | 9.97 |
| S | Serine | 105.09 | 52.55 | 50 | 100 | 10.00 |
| G | Glycine | 75.07 | 38.56 | 50 | 100 | 10.27 |
| R | Arginine HCl | 210.66 | 102.67 | 50 | 100 | 9.75 |
| N-iSTD | Norvaline | 117.15 | 58.76 | 50 | 100 | 10.03 |
| S-iSTD | Sarcosine | 89.09 | 44.00 | 50 | 98.4 | 9.72 |
| P | Proline | 115.13 | 57.74 | 50 | 100 | 10.03 |

Weigh the calculated weight into a 50-mL volumetric flask
Dissolve and bring to mark with 0.1N HCl
Mix well by capping and inverting or vortexing
Store at 2-8° C. until HPLC analysis (step 3.6)
   Internal Standard Mixture: 1 μmol/mL
Into the same 10-mL volumetric flask, pipet accurately 1.0 mL of Norvaline stock (10 μmol/mL) and 1.0 mL of Sarcosine stock (10 μmol/mL) solutions
Bring to mark with 0.1N HCl
Mix well by capping and inverting or vortexing
Store at 2-8° C. until HPLC analysis (step 3.6)
   Internal Standard Mixture: 0.1 μmol/mL
   NOTE: this solution is for reconstituting samples after drying
Into a 10-mL volumetric flask, pipet accurately 1.0 mL of the internal standard mixture (1 μmol/mL)
Bring to mark with 0.1N HCl
Mix well by capping and inverting or vortexing
Store at 2-8° C. until HPLC analysis (step 3.6)
   5 AA Mixture (+iSTD 0.1): 0.025/0.1/0.25 μmol/mL
Into the same 10-mL volumetric flask, pipet accurately xx μL (see table below) of D, S, G, R, N-iSTD, S-iSTD, and P (10 μmol/mL) solutions
Bring to mark with 0.1N HCl
Mix well by capping and inverting or vortexing
Store at 2-8° C. until HPLC analysis (step 3.6)

| | | N-iSTD | N-iSTD | | Final | |
|---|---|---|---|---|---|---|
| D/S/G/R/P | | or | or | | μmol/mL | Final |
| STD | AASTD | S-iSTD | S-iSTD | Total | (D/S/G/ | μmol/mL |
| (μmol/mL) | (xx μL) | (μmol/mL) | (xx μL) | (mL) | R/P) | (iSTD) |
| 10 | 25 | 10 | 100 | 10 | 0.025 | 0.1 |
| 10 | 100 | 10 | 100 | 10 | 0.1 | 0.1 |
| 10 | 250 | 10 | 100 | 10 | 0.25 | 0.1 |

17 AA Standard (+iSTD 0.1) Mixture: 0.1 μmol/mL
Break open an ampoule of the 0.1 μmol/mL 17 AA standard solution
A Accurately pipet 0.9 mL of the AA standard mixture into an LC vial
A Into the same LC vial, pipet accurately 100 μL of the iSTD mixture (1 μmol/mL)
Mix well by vortexing
Aliquot NLT 100 μL into an LC vial with an LC vial insert
Store at 2-8° C. until HPLC analysis (step 3.6)

### HPLC Conditions

| Instrument | Agilent 1260 LC with UV and Fluorescence detector | | |
|---|---|---|---|
| Column | AdvanceBio AAA LC, 2.7 μm, 4.6 × 100 mm | | |
| | Agilent 655950-802 | | |
| Gulard column | AdvanceBio AAA guard column, 2.7 μm, 4.6 × 5 mm | | |
| | Agilent 820750-931 | | |
| Mobile phase aqueous | 10 mM Na₂HPO₄ 10 mM Na₂B₄O₇ (pH 8.2) | | |
| Mobile phase organic | 45/45/10 ACN/MeOH/water | | |
| Flow rate | 1.5 mL/min | | |
| | Minute | % Aqueous | % Organic |
| Gradient | 0.0 | 98 | 2 |
| | 0.35 | 98 | 2 |
| | 13.4 | 43 | 57 |
| | 13.5 | 0 | 100 |
| | 15.7 | 0 | 100 |
| | 15.8 | 98 | 2 |
| | 18 | 98 | 2 |

| Column temperature | 40° C. | |
|---|---|---|
| Injection | l μL | |
| Needle wash | Flush port for 7 s | |
| | Function | Parameter |
| Online derivatization | Draw | Draw 2.5 μL from location “1” with default speed using |
| (Use Injector | | default offset (borate buffer) |
| Program) | Draw | Draw l μL from sample with default speed using default |
| | | offset |
| | Mix | Mix 3.5 μL from seat with default speed for 5 times |
| | Wait | Wait 0.2 min |
| | Draw | Draw 0.5 μL from location “2” with default speed using |
| | | default offset (OPA) |
| | Mix | Mix 4 μL from seat with default speed for 10 times |
| | Draw | Draw 0.4 μL from location “3” with default speed using |
| | | default offset (FMOC) |
| | Mix | Mix 4.4 μL from seat with default speed for 10 times |
| | Draw | Draw 32 μL from location “4” with default speed using |
| | | default offset (Injection diluent) |
| | Mix | Mix 20 μL from seat with default speed for 8 times |
| | Inject | Inject |
| | Wait | Wait 0.1 min |
| | Valve | Switch valve to “Bypass” |

| UV | Response time: 1 s | | | |
|---|---|---|---|---|
| | Autobalance: prerun | | | |
| | Slit: 4 nm | | | |
| | Wavelength | Bandwith | Reference | Bandwith |
| | 338 nm | 10 nm | 390 nm | 20 nm |
| | Switch between the last eluting OPA-derivatized AA (Lysine) and | | | |
| | before the 1^{st} eluting FMOC-derivatizedAA (Hydroxyproline): ~11 | | | |
| | min | | | |
| | 262 nm | 16 nm | 324 nm | 8 nm |

| FLR | Response time: 1 s | |
|---|---|---|
| | PMT gain: 10 (adjust if needed) | |
| | Excitation | Emission |
| | 340 nm | 450 nm |
| | Switch between the last eluting OPA-derivatized AA (Lysine) and | |
| | before the 1^{st} eluting FMOC-derivatizedAA (Hydroxyproline): ~ | |
| | 11 min | |
| | 260 nm | 325 |

System suitability criteria
Analyze retention time and peak area for each AA of interest used in quantitation (D/S/G/R) and internal standards (Norvaline and Sarcosine) in standard injections (both UV and FLR).

| Parameter | Criteria |
|---|---|
| Blanks | No significant interference |
| | in UV and FLR |
| % RSD (Retention time), initial 3 | NMT 5% |
| % RSD (Area), initial 3 | NMT 20% |
| % RSD (Relative Area), initial 3 | NMT 20% |
| % RSD (Retention time), all | NMT 5% |
| % RSD (Area), all | NMT 20% |
| % RSD (Relative Area), all | NMT 20% |
| Resolution: | Baseline separation |
| Glycine from other AAs | |
| Norvaline (iSTD) from other AAs | |
| Sarcosine (iSTD) vs. Proline | |
| Linearity | RSQ NLT 0.99 |
| Check standard | Quantitated result: within |
| | 80%~120% of theoretical |

Data Analysis—Analyze Samples Only when System Suitability Passes

### Identification of AA of Interest

RT of AA of interest and internal standards in the 17AA (+iSTD) standard mixture should match the RT of the 5AA (+iSTD) standard mixture
RT of the AA in each sample should match the RT of the standard (UV/FLR)

Relative area (D,S,G,R)=Area (D,S,G,R)/Area (Norvaline)

Relative area (P)=Area (P)/Area (Sarcosine)

Standard calibration curve
Calculate concentrations of standard solutions using the reported value on certificate of analysis or actual weights adjusted by dilution factor during the standard preparation.
Plot the average area or average relative area vs. concentration for each AA of interest from the standard injections: 0.025, 0.1, and 0.25 μmol/mL. Perform linear regression.

Conc=m*Area or Relative Area+b
Where, m is the slope of the linear fitted curve and b is the Y-intercept of the linear fitted curve.
The RSQ should be no less than 0.99.
If the linearity fails, prepare fresh derivatization reagents/standard solutions, and troubleshoot system malfunction and repeat the test.

### Sample Analysis

Quantitation can be done by both UV and FLR
Quantitation is done using relative area (area ratio relative to the internal standard)
Calculate concentration of AA: G, R, D, S in each sample using the linear fitted standard curve:

Conc,AA (μmol/mL)=(m*Area or Relative Area+b)
Calculate concentration of the total GRGDSP (SEQ ID NO: 49) by averaging the concentration of each AA:

Concentration,total GRGDSP (SEQ ID NO: 49) (μmol/mL)=(Conc,G/2+Conc,R+Conc,D+Conc,S+Conc,P)/5

μmol (Total GRGDSP (SEQ ID NO: 49))/g (conjugate)=Conc,total GRGDSP (SEQ ID NO: 49) (μmol/mL)×0.25 mL/1.0 mL×20 mL/Weight (g)

μmol (conjugated GRGDSP (SEQ ID NO: 49))/g (conjugate)=μmol (Total GRGDSP (SEQ ID NO: 49))/g (conjugate)−μmol (Residual free GRGDSP (SEQ ID NO: 49))/g (conjugate)

### Example 8: Exemplary Assay to Determine Residual Free Peptide in a CBP-Polymer Composition

This assay uses liquid chromatography—mass spectroscopy (LC-MS) to determine the amount of residual, unconjugated peptide in a composition containing a peptide-polymer conjugate, e.g., typically after one or more purification steps have been performed to remove a substantial portion, e.g., greater than 95%, 98%, 99% or more, of unconjugated peptide. In brief, a sample of the conjugate in saline solution is added to a molecular weight cut-off (MWCO) tube that has a MWCO higher than the molecular weight of the peptide, the tube is centrifuged to separate the residual peptide from the conjugate, and the amount of peptide is quantitated by LC-MS using as a standard a reference composition containing a known concentration of the same peptide.

### Example 9: Exemplary Quantitative Amine Assay to Determine Amine-Conjugation Density in an Afibrotic Polymer Modified with a Compound of Formula (I)

This assay determines the amount of an amine-containing compound (e.g., a compound of Formula I, e.g., Compound 101 in Table 3) in a polymer chemically modified with the amine compound. A sample of the chemically-modified polymer is subjected to acid hydrolysis, which cleaves off the conjugated amine and the weight % of total amine in the hydrolyzed sample is quantitated by reverse-phase, liquid chromatography with ultraviolet detection (LC-UV) using the unconjugated amine compound as a standard. The identity of the LC peak can be further confirmed by mass spectrometry. The weight % of total amine can be used as the % conjugation of the amine-compound in the chemically-modified polymer. A more precise result can be obtained by determining the amount of any residual unconjugated amine compound in an unhydrolyzed sample of the chemically-modified polymer using any suitable method (e.g., as described below) and subtracting that amount from the total peptide amount.

The assay is further described below as applied to determining % conjugation density in an alginate chemically modified with Compound 101 (i.e., CM-LMW-Alg-101); however, the skilled artisan can readily modify the assay to determine the conjugation density of any Formula I compound used to chemically-modify a polysaccharide (e.g., an alginate) or another polymer that does not contain amines. Also, the skilled person can readily substitute any equipment, material or chemical specified below with a different equipment, material or chemical that can perform or provide substantially the same function or role in the assay.

### Definitions

| Abbreviation | Definition |
|---|---|
| LC-UV-MS | Liquid Chromatography-Ultra-Violet-Mass |
| | Spectrometry |
| VLVG | Pronova Ultrapure VLVG sodium alginate |
| SLG100 | Pronova Ultrapure Sterile Alginate |
| TIC | Total Ion Chromatogram |
| SST | System Suitability |
| RSD | Relative Standard Deviation |
| m/z | Mass charge ratio |

### Equipment

Agilent 1260 LC system (DAD: 13 μL/10 mm flow cell)
Agilent SQ MS detector (G1956B)
XBridge C18, 2.5 μm, 4.6×50 mm, Waters 186006037
Small molecule reference material (unconjugated, free amine version of Compound 101 in Table 3; >98.0% purity)

### Procedure

Prepare a 0.1% ammonia in water solution (aqueous mobile phase) and a 0.1% ammonia in ACN solution (organic mobile phase) for use in the LC procedure.

### Acid Hydrolysis of an Exemplary Solid Small Molecule-Alginate Conjugate Sample

Weigh 50±5 mg of the lyophilized small molecule-alginate conjugate solid, into a microwave reaction vial, ensuring the sample is not agitate
Add 10.0 mL of 2N HCl using a 10-mL transfer pipet or volumetric pipet and a stir bar, and seal the PTFE-lined cap with a crimper
Place each sample vial in the matching heat block on a hot/stir plate and heat at 120° C., stirring at 400 rpm for 120 minutes
Remove from heat and let cool to ambient temperature
Transfer the entire solution from the reaction vial to a 25-mL volumetric flask with a disposable transfer pipet
Pipet 5 mL of LCMS grade water into the empty reaction vial, rinse the inner wall thoroughly with the same disposable transfer pipet, and transfer the rinseate completely to the same 25-mL volumetric flask
Repeat the above step twice
Bring to mark of the volumetric flask with LCMS grade water
Transfer completely to a 50-mL centrifuge tube
Centrifuge at 3000 rpm for 10 minutes
Take supernatant for HPLC analysis
Store at 2-8° C.

### Acid Hydrolysis of an Exemplary Small Molecule-Alginate Conjugate in Saline Sample

Weigh 1000±50 mg of the small molecule-alginate conjugate solution in saline into a microwave reaction vial
Add 10.0 mL of 2N HCl using a 10-mL transfer pipet or volumetric pipet and a stir bar, and seal the PTFE-lined cap with a crimper
Place each sample vial in the matching heat block on a hot/stir plate
Heat at 120° C., stir at 400 rpm, for 120 minutes
Remove from heat and let cool to ambient temperature
Transfer the entire solution from the reaction vial to a 25-mL volumetric flask with a disposable transfer pipet
Pipet 5 mL of LCMS grade water into the empty reaction vial, rinse the inner wall thoroughly with the same disposable transfer pipet, and transfer the rinseate completely to the same 25-mL volumetric flask
Repeat the above step twice
Bring to mark of the volumetric flask with LCMS grade water
Transfer completely to a 50-mL centrifuge tube
Centrifuge at 3000 rpm for 10 minutes
Take supernatant for HPLC analysis
Store at 2-8° C.

### Sample Preparation for Residual Free Amine in Solid Small Molecule-Alginate Conjugate

Weigh 50±5 mg of the lyophilized small molecule-alginate conjugate solid, into a scintillation vial
Pipet 5.0 mL of saline into the scintillation vial
Dissolve completely by shaking and vortexing for 10 minutes
Transfer completely to a MWCO tube
Centrifuge at 5000 rpm for 60 minutes
Remove the top portion of the MWCO tube and discard
Transfer the sample in the bottom portion completely to a 5 mL volumetric flask
Bring to mark with water or saline and invert to mix well
Transfer to a scintillation vial for storage at 2-8° C.
Transfer an aliquot for HPLC analysis

### Sample Preparation for Residual Free Amine in Small Molecule-Alginate Conjugate in Saline

Weigh 1000±50 mg of the small molecule-alginate conjugate (or blend with unmodified alginate) in saline, into a MWCO tube
Pipet 4.0 mL of saline into the MWCO tube
Invert and vortex the tube 5 times or until the solution is mixed well to fully extract free amine
Centrifuge at 5000 rpm for 90 minutes
Remove the top portion of the MWCO tube and discard
Transfer the sample in the bottom portion completely to a 5 mL volumetric flask
Bring to mark with water and invert to mix well
Transfer to a scintillation vial for storage at 2-8° C.
Transfer an aliquot for HPLC analysis

### Standard Preparation

Standard Solution: 1 mg/mL
Weigh 50.00±5.00 mg of the small molecule reference material standard into a scintillation vial
Add ˜10 mL of LCMS-grade water, dissolve the solid completely by shaking and vortexing
Transfer completely to a 50-mL volumetric flask by rinsing the scintillation vial twice with LCMS-grade water, using a disposable transfer pipet
Bring to volume with LCMS-grade water, mix well
Store at 2-8° C.
   Standard Solution: 0.01 mg/mL
Pipet 100 μL of the 1 mg/mL solution to a 10-mL volumetric flask
Bring to volume with LCMS-grade water
Mix well by inverting
Store at 2-8° C.

### HPLC Conditions

| Instrument | Agilent 1260 LC with DAD and SQ MS (optional) | | |
|---|---|---|---|
| Column | XBridge C18, 2.5 μm, 4.6 × 50 mm | | |
| Mobile phase aqueous | 0.1% ammonia | | |
| Mobile phase organic | 0.1% ammonia in ACN | | |
| Flow rate | 1.0 mL/min | | |
| | Minute | % Aqueous | % Organic |
| Gradient | 0.0 | 98 | 2 |
| | 6.0 | 86 | 14 |
| | 12.0 | 20 | 80 |
| | 12.1 | 98 | 2 |
| | 15.0 | 98 | 2 |

| | |
|---|---|
| Column temperature | 30° C. |
| Injection | 10 μL |
| UV | Detection: 220 nm, bw 10 nm; |
| | Reference: 360 nm, bw 100 nm; |
| | Response time: 1 s |
| | Autobalance: prerun |
| | Slit: 4 nm |
| MS (optional) | API-ES (scan: positive and negative) |
| | Drying gas: 12 L/min; Nebulizer pressure: 55 psig; |
| | Drying gas temperature: 350° C.; |
| | Capillary Voltage: 3000 V; Scan range 90-1000; |
| | Fragmentor 70 V; Gain 1.00; Threshold 150; Step size 0.10 |

### System Suitability Criteria

| Parameter | Criteria |
|---|---|
| Blanks | No significant interference |
| | in UV and TIC (optional) |
| % RSD (Retention time), initial 5 | NMT 2% |
| Small molecule reference material | |
| % RSD (area), initial 5 | NMT 10% |
| Small molecule reference material | |
| % RSD (Retention time), initial 5 and all | NMT 2% |
| bracketing Small molecule reference | |
| material | |
| % RSD (area), initial 5 and all bracketing | NMT 10% |
| Small molecule reference material | |
| m/z: amine peak (optional) | 392.1 ± 0.5 |

Data Analysis—Analyze Samples Only when System Suitability Passes

### Identification of Amine

(optional) m/z of the free amine peak in each sample should be within 392.1±0.5.
RT of the amine peak in UV in each sample matches the RT of the standard.

Concentration,standard (mg/mL)=Weight (mg)/50 mL/dilution factor,
Where dilution factor=1 for 1.0 mg/mL standard;
Where dilution factor=100 for 0.01 mg/mL standard

Concentration,free amine (mg/mL)=Area,non-hydrolyzed sample/Area,0.01 standard×Concentration,0.01 standard

% Residual free amine=Concentration,free amine (mg/mL)×5 mL/weight,non-hydrolyzed conjugate (mg)×100

Concentration,total amine (mg/mL)=Area,hydrolyzed sample/Area,1.0 standard×Concentration,1.0 standard

% Total amine=Concentration,total amine (mg/mL)×25 mL/weight,hydrolyzed conjugate (mg)×100

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. An engineered RPE cell capable of expressing and secreting a factor VII (FVII) protein, wherein the engineered RPE cell comprises an exogenous nucleotide sequence comprising a coding sequence for the FVII protein, wherein the coding sequence comprises a precursor FVII coding sequence selected from the group consisting of:
(i) SEQ ID NO:3,
(ii) a nucleotide sequence that is at least 95% identical to SEQ ID NO:3,
(iii) SEQ ID NO:4, and
(iv) a nucleotide sequence that is at least 95% identical to SEQ ID NO:4.

2. The engineered RPE cell of claim 1, wherein the exogenous nucleotide sequence further comprises a promoter operably linked to the coding sequence for the FVII protein, wherein the promoter comprises the nucleotide sequence of SEQ ID NO:10 or SEQ ID NO:21.

3. The engineered RPE cell of claim 2, wherein the precursor FVII coding sequence comprises SEQ ID NO:3 and the promoter consists of SEQ ID NO:10 or SEQ ID NO:21.

4. The engineered RPE cell of claim 1, wherein the FVII protein is an FVII fusion protein.

5. The engineered RPE cell of claim 4, wherein the coding sequence for the FVII protein comprises SEQ ID NO:13, SEQ ID NO:14, SEQ ID NO:15, SEQ ID NO:16, SEQ ID NO:17 or SEQ ID NO:18.

6. The engineered RPE cell of claim 1, wherein the exogenous nucleotide sequence further comprises SEQ ID NO:23, SEQ ID NO:24 or SEQ ID NO:25.

7. The engineered RPE cell of claim 1, wherein the exogenous nucleotide sequence further comprises SEQ ID NO:22.

8. The engineered RPE cell of claim 1, wherein the exogenous nucleotide sequence comprises an extrachromosomal expression vector or is integrated into at least one chromosomal location in the RPE cell.

9. The engineered RPE cell of claim 1, wherein the engineered RPE cell is derived from an ARPE-19 cell.

10. A composition comprising an engineered RPE cell of claim 1.

11. The composition of claim 10, wherein the composition comprises a polyclonal cell culture or a culture of a monoclonal cell line.

12. An isolated double-stranded DNA molecule which comprises a nucleotide sequence selected from the group consisting of: SEQ ID NO:3, SEQ ID NO:4, SEQ ID NO:13, SEQ ID NO:14, SEQ ID NO:15, SEQ ID NO:16, SEQ ID NO:17, SEQ ID NO:18, SEQ ID NO:22, SEQ ID NO:23, SEQ ID NO:24 and SEQ ID NO:25.

13. The isolated DNA molecule of claim 12, which consists essentially of SEQ ID NO:22.

14. An implantable device comprising at least one cell-containing compartment which comprises an engineered RPE cell of claim 1 and at least one means for mitigating the foreign body response (FBR) when the device is implanted into the subject.

15. The implantable device of claim 14, wherein the at least one cell-containing compartment comprises a polymer composition which encapsulates the plurality of engineered RPE cells.

16. The implantable device of claim 14, wherein the cell-containing compartment is surrounded by a barrier compartment comprising an alginate hydrogel and optionally a compound of Formula (I) disposed on the outer surface of the barrier compartment.

17. The implantable device of claim 16, wherein the polymer composition comprises an alginate covalently modified with a peptide, wherein the peptide comprises GRGDSP (SEQ ID NO:49) or GGRGDSP (SEQ ID NO:51), and wherein the barrier compartment comprises an alginate modified with 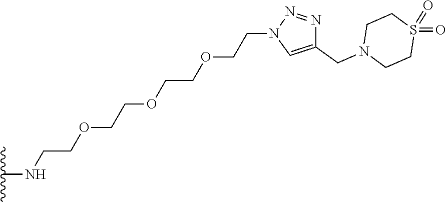 [SMILES: *NCCOCCOCCOCCn1cc(CN2CCS(=O)(=O)CC2)nn1]
or a pharmaceutically acceptable salt thereof.

18. A hydrogel capsule comprising:
(a) an inner compartment which comprises an engineered cell of claim 1 encapsulated in a first polymer composition, wherein the first polymer composition comprises a hydrogel-forming polymer; and
(b) a barrier compartment surrounding the inner compartment and comprising a second polymer composition, wherein the second polymer composition comprises an alginate covalently modified with at least one compound selected from the group consisting of Compound 100, Compound 101, Compound 110, Compound 112, Compound 113 and Compound 114 as shown in the table below:

| Compound No. | Structure |
|---|---|
| 100 | 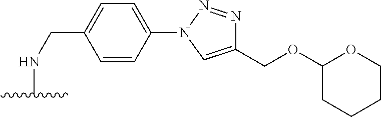 [SMILES: *NCc1ccc(-n2cc(COC3CCCCO3)nn2)cc1] |
| 101 | 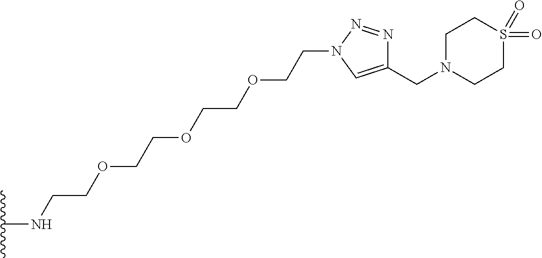 [SMILES: *NCCOCCOCCOCCn1cc(CN2CCS(=O)(=O)CC2)nn1] |
| 110 | 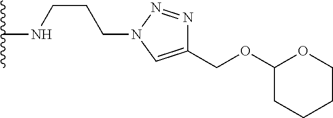 [SMILES: *NCCCn1cc(COC2CCCCO2)nn1] |
| 112 | 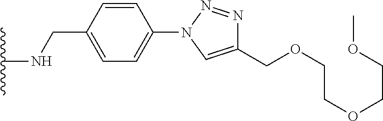 [SMILES: *NCc1ccc(-n2cc(COCCOCCOC)nn2)cc1] |
| 113 | 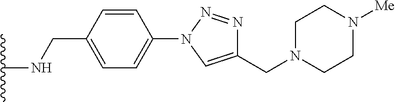 [SMILES: *NCc1ccc(-n2cc(CN3CCN(C)CC3)nn2)cc1] |
| 114 | 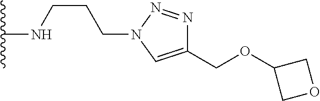 [SMILES: *NCCCn1cc(COC2COC2)nn1] |

or a pharmaceutically acceptable salt of the compound.

19. The hydrogel capsule of claim 18, wherein the selected compound is 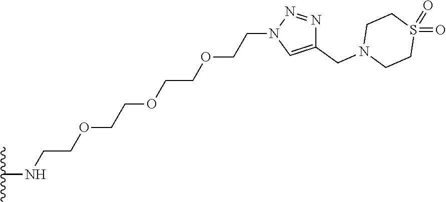 [SMILES: *NCCOCCOCCOCCn1cc(CN2CCS(=O)(=O)CC2)nn1]
or a pharmaceutically acceptable salt thereof.

20. The hydrogel capsule of claim 18, wherein the inner compartment comprises a plurality of the engineered cells.

21. The hydrogel capsule of claim 18, wherein the engineered cell is derived from an ARPE-19 cell and comprises SEQ ID NO:25.

22. A composition comprising a plurality of the hydrogel capsules of claim 18.

23. A method of delivering a FVII therapy to a patient in need thereof, comprising administering the device of claim 14.

24. The engineered RPE cells of claim 1, wherein the FVII protein comprises SEQ ID NO: 11 or SEQ ID NO: 12.

25. An isolated polynucleotide comprising a coding sequence for a factor VII (FVII) protein, wherein the polynucleotide has at last one or more of the following features:
(a) a promoter operably linked to the coding sequence, wherein the promoter comprises the nucleotide sequence of SEQ ID NO:10 or SEQ ID NO:21; and
(b) the coding sequence comprises a precursor FVII coding sequence selected from the group consisting of:
(i) SEQ ID NO:3,
(ii) a nucleotide sequence that is at least 95% identical to SEQ ID NO:3,
(iii) SEQ ID NO:4, and
(iv) a nucleotide sequence that is at least 95% identical to SEQ ID NO:4.

26. The isolated polynucleotide of claim 25, which comprises SEQ ID NO:10 as a promoter operably linked to the precursor FVII coding sequence.

27. The isolated polynucleotide of claim 25, wherein the FVII protein comprises SEQ ID NO:11 or SEQ ID NO:12.

28. The isolated polynucleotide of claim 25, wherein the coding sequence comprises a sequence selected from SEQ ID NO:13, SEQ ID NO:14, SEQ ID NO:15, SEQ ID NO:16, SEQ ID NO:17 and SEQ ID NO:18.

29. An isolated polynucleotide comprising an upstream transcription unit and a downstream transcription unit, wherein the upstream transcription unit comprises SEQ ID NO:10 operably linked to SEQ ID NO:3 and the downstream transcription unit comprises SEQ ID NO:21 operably linked to SEQ ID NO:3.

30. An isolated double-stranded DNA molecule which comprises a nucleotide sequence selected from the group consisting of: SEQ ID NO:3, SEQ ID NO:4, SEQ ID NO:13, SEQ ID NO:14, SEQ ID NO:15, SEQ ID NO:16, SEQ ID NO:17, SEQ ID NO:18, SEQ ID NO:22, SEQ ID NO:23, SEQ ID NO:24 and SEQ ID NO:25.
